(19) United States
(12) Patent Application Publication
(10) Pub. No.: US 2024/0197778 A1
(43) Pub. Date: Jun. 20, 2024

(54) TREATMENT OF CANCER WITH NK CELLS AND A HER2 TARGETED ANTIBODY

(71) Applicants: Artiva Biotherapeutics, Inc., San Diego, CA (US); GC Cell Corporation, Yongin-si Gyeonggi-do (KR)

(72) Inventors: Yusun KIM, Yongin-si, Gyeonggi-do (KR); Sungyoo CHO, Yongin-si, Gyeonggi-do (KR); Yu Kyeong HWANG, Yongin-si, Gyeonggi-do (KR); Bokyung MIN, Yongin-si, Gyeonggi-do (KR); Bitna YANG, Yongin-si, Gyeonggi-do (KR); Eunji KIM, Yongin-si, Gyeonggi-do (KR); Peter FLYNN, Cardiff by the Sea, CA (US); Jason B. LITTEN, Poway, CA (US); Thomas James FARRELL, La Jolla, CA (US); John Kin Chuan LIM, San Diego, CA (US); Mili MANDAL, San Diego, CA (US)

(21) Appl. No.: 18/285,647

(22) PCT Filed: Apr. 6, 2022
(86) PCT No.: PCT/US2022/023684

§ 371 Date: Oct. 4, 2023

Related U.S. Application Data
(60) Provisional application No. 63/172,414, filed on Apr. 8, 2021.
(60) Provisional application No. 63/290,359, filed on Dec. 16, 2021.

(51) Int. Cl. A61K 35/17; A61K 31/664; A61K 31/7076; A61K 38/20; A61K 39/00; A61P 35/00; C07K 16/32; C12N 5/0783

(52) U.S. Cl. CPC A61K 35/17; A61K 31/664; A61K 31/7076; A61K 38/2013; A61K 39/4613; A61P 35/00; C07K 16/32; C12N 5/0646; A61K 2039/505; A61K 2239/38; A61K 2239/49; A61K 2239/51; A61K 2239/59

(57) ABSTRACT

Provided herein are, among other things, methods for treating a patient suffering from a HER2+ cancer.

### CLAIM OF PRIORITY

This application claims the benefit of U.S. Provisional Application Ser. No. 63/290,359, filed on Dec. 16, 2021, and U.S. Provisional Application Ser. No. 63/172,414, filed on Apr. 8, 2021. The entire contents of the foregoing are incorporated herein by reference.

### BACKGROUND

Targeted therapies, including antibody therapy, have revolutionized cancer treatment. One mechanism of action by which antibody therapy induces cytotoxicity is through antibody dependent cell-mediated cytotoxicity (ADCC). Many cancer patients are unable to mount a robust ADCC response. A reduced ADCC response may render any of the indicated monoclonal antibody therapeutics significantly less effective for these patients, which could prevent these patients from responding or lead to relapse. Thus, a reduced ADCC response could negatively impact their clinical outcomes.

Despite recent discoveries and developments of several anti-cancer agents, there is still a need for improved methods and therapeutic agents due to poor prognosis for many types of cancers, including HER2+ cancers.

The present invention addresses these and other deficiencies in the art.

### SUMMARY

NK cells are immune cells that can engage tumor cells through a complex array of receptors on their cell surface, as well as through antibody-dependent cellular cytotoxicity (ADCC). To initiate ADCC, NK cells engage with antibodies via the CD16 receptor on their surface. NK cells may have an advantage over other immune cells, such as the T cells used in CAR-T cell therapy and other cell therapies. In an exemplary advantage, NK cells can be used as allogeneic therapies, meaning that NK cells from one donor can be safely used in one or many patients without the requirement for HLA matching, gene editing, or other genetic manipulations. Allogeneic NK cells with anti-tumor activity can be administered safely to patients without many of the risks associated with T cell therapies, such as severe cytokine release syndrome (CRS), and neurological toxicities or graft versus host disease (GvHD).

Allogeneic NK cells may provide an important treatment option for cancer patients. In one exemplary advantage, NK cells have been well tolerated without evidence of graft-versus-host disease, neurotoxicity or cytokine release syndrome associated with other cell-based therapies. In another exemplary advantage, NK cells do not require prior antigen exposure or expression of a specific antigen to identify and lyse tumor cells. In another exemplary advantage, NK cells have the inherent ability to bridge between innate immunity and engender a multi-clonal adaptive immune response resulting in long-term anticancer immune memory. All of these features contribute to the potential for NK cell efficacy as cancer treatment options.

For example, NK cells can recruit and activate other components of the immune system. Activated NK cells secrete cytokines and chemokines, such as interferon gamma (IFNγ); tumor necrosis factor alpha (TNFα); and macrophage inflammatory protein 1 (MIP1) that signal and recruit T cells to tumors. Through direct killing of tumor cells, NK cells also expose tumor antigens for recognition by the adaptive immune system.

Additionally, cords with preferred characteristics for enhanced clinical activity (e.g., high-affinity CD16 and Killer cell Immunoglobulin-like Receptor (KIR) B-haplotype) can be selected by utilizing a diverse umbilical cord blood bank as a source for NK cells.

The administration of the allogenic NK cells, as described herein, can enhance patients' ADCC responses, e.g., when undergoing monoclonal antibody therapy. In one exemplary advantage, the allogeneic NK cells described herein can be used even in patients who have developed full or partial resistance to antibodies. For example, the HER2 pathway typically promotes cell growth and division. In some cancers, HER2 expression is upregulated, thus promoting constant proliferation and tumor formation. The HER2 pathway initiates the MAP kinase and PI3 kinase/AKT pathways. Trastuzumab can interrupt the dimerization of HER2, thereby inhibiting HER2 activation and downstream signaling. However, mutations in these pathways can cause constitutive activation even in the absence of HER2 signaling, thereby providing a mechanism for trastuzumab resistance. In an exemplary advantage, the NK cells described herein can still be used in combination with some trastuzumab-resistant cancers because they mediate ADCC, which can kill tumor cells independently of HER2 signaling. In such cases, the NK cells can still bind to trastuzumab antibody bound to HER2 on the surface of cells and mediate cell killing.

Provided herein, among other things, are methods for treating a patient suffering from a HER2+ cancer.

Provided herein are methods for treating a patient suffering from a HER2+ cancer, comprising administering a population of natural killer cells (NK cells) and an antibody targeted to human HER2, wherein the NK cells are allogenic to the patient, are KIR-B haplotype and homozygous for a CD16 158V polymorphism.

In some embodiments, the cancer is selected from the group consisting of breast cancer, gastric cancer, and ovarian cancer.

In some embodiments, the cancer is breast cancer.

In some embodiments, the cancer is gastric cancer.

In some embodiments, the cancer is ovarian cancer.

In some embodiments, the patient has relapsed after treatment with an anti-HER2 antibody.

In some embodiments, the patient has experienced disease progression after treatment with autologous stem cell transplant or chimeric antigen receptor T-cell therapy (CAR-T).

In some embodiments, the patient is administered 1×10⁸ to 1×10¹¹ NK cells.

In some embodiments, the patient is administered 1×10⁹ to 8×10⁹ NK cells.

In some embodiments, the patient is administered 4×10⁸, 1×10⁹, 4×10⁹, or 8×10⁹ NK cells.

In some embodiments, the antibody is trastuzumab.

In some embodiments, the patient is subjected to lymphodepleting chemotherapy prior to treatment.

In some embodiments, the lymphodepleting chemotherapy is non-myeloablative chemotherapy.

In some embodiments, the lymphodepleting chemotherapy comprises treatment with at least one of cyclophosphamide and fludarabine.

In some embodiments, the lymphodepleting chemotherapy comprises treatment with cyclophosphamide and fludarabine.

In some embodiments, the cyclophosphamide is administered between 100 and 500 mg/m²/day.

In some embodiments, the cyclophosphamide is administered at 250 mg/m²/day.

In some embodiments, the cyclophosphamide is administered at 500 mg/m²/day.

In some embodiments, the fludarabine is administered between 10 and 50 mg/m²/day.

In some embodiments, the fludarabine is administered 30 mg/m²/day.

In some embodiments, the method further comprises administering IL-2.

In some embodiments, the patient is administered 1×10⁶ IU/m² of IL-2.

In some embodiments, the patient is administered 6 million IU of IL-2.

In some embodiments, administration of IL-2 occurs within 1-4 hrs of administration of the NK cells.

In some embodiments, administration of the NK cells and the antibody targeted to human HER2 occurs weekly.

In some embodiments, the NK cells and the antibody targeted to human HER2 are administered weekly for 4 to 8 weeks.

In some embodiments, the administration of the NK cells occurs weekly and the administration of the antibody targeted to human HER2 occurs every other week.

In some embodiments, the NK cells are not genetically modified.

In some embodiments, at least 70% of the NK cells are CD56+ and CD16+.

In some embodiments, at least 85% of the NK cells are CD56+ and CD3−.

In some embodiments, 1% or less of the NK cells are CD3+, 1% or less of the NK cells are CD19+ and 1% or less of the NK cells are CD14+.

In some embodiments, the each administration of NK cells is administration of 1×10⁹ to 5×10⁹ NK cells.

In some embodiments, the patient receives a dose of the HER2 targeting antibody before the first dose of NK cells.

In some embodiments, the expanded natural killer cells are expanded umbilical cord blood natural killer cells.

In some embodiments, the population of expanded natural killer cells comprises at least 60%, e.g., at least 70%, at least 80%, at least 90% at least 95%, at least 99%, or 100% CD16+ cells.

In some embodiments, the population of expanded natural killer cells comprises at least 60%, e.g., at least 70%, at least 80%, at least 90% at least 95%, at least 99%, or 100% NKG2D+ cells.

In some embodiments, the population of expanded natural killer cells comprises at least 60%, e.g., at least 70%, at least 80%, at least 90% at least 95%, at least 99%, or 100% NKp46+ cells.

In some embodiments, the population of expanded natural killer cells comprises at least 60%, e.g., at least 70%, at least 80%, at least 90% at least 95%, at least 99%, or 100% NKp30+ cells.

In some embodiments, the population of expanded natural killer cells comprises at least 60%, e.g., at least 70%, at least 80%, at least 90% at least 95%, at least 99%, or 100% DNAM-1+ cells.

In some embodiments, the population of expanded natural killer cells comprises at least 60%, e.g., at least 70%, at least 80%, at least 90% at least 95%, at least 99%, or 100% NKp44+ cells.

In some embodiments, the population of expanded natural killer cells comprises less than 20%, e.g., 10% or less, 5% or less, 1% or less, 0.5% or less, or 0% CD3+ cells.

In some embodiments, the population of expanded natural killer cells comprises less than 20% or less, e.g., 10% or less, 5% or less, 1% or less, 0.5% or less, or 0% CD14+ cells.

In some embodiments, the population of expanded natural killer cells comprises less than 20% or less, e.g., 10% or less, 5% or less, 1% or less, 0.5% or less, or 0% CD19+ cells.

In some embodiments, the population of expanded natural killer cells comprises less than 20% or less, e.g., 10% or less, 5% or less, 1% or less, 0.5% or less, or 0% CD38+ cells.

In some embodiments, the natural killer cells do not comprise a CD16 transgene.

In some embodiments, the natural killer cells do not express an exogenous CD16 protein.

In some embodiments, the expanded natural killer cells are not genetically engineered.

In some embodiments, the expanded natural killer cells are derived from the same umbilical cord blood donor.

In some embodiments, the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the population of NK cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce expanded natural killer cells, thereby producing the population of expanded natural killer cells.

In some embodiments, the population of NK cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and (d) expanding the master cell bank population of expanded natural killer cells by culturing with a second plurality of Hut78 cells engineered to express a membrane bound IL-21, a mutated TNFα, and a 4-1BBL gene to produce expanded natural killer cells; thereby producing the population of expanded natural killer cells.

In some embodiments, the population of NK cells is produced by a method further comprising, after step (c), (i) freezing the master cell bank population of expanded natural killer cells in a plurality of containers; and (ii) thawing a container comprising an aliquot of the master cell bank population of expanded natural killer cells, wherein expanding the master cell bank population of expanded natural killer cells in step (d) comprises expanding the aliquot of the master cell bank population of expanded natural killer cells.

In some embodiments, the umbilical cord blood is from a donor with the KIR-B haplotype and homozygous for the CD16 158V polymorphism.

In some embodiments, the population of NK cells is produced by a method comprising expanding the natural killer cells from umbilical cord blood at least 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold.

In some embodiments, the population of expanded natural killer cells is not enriched or sorted after expansion.

In some embodiments, the percentage of NK cells expressing CD16 in the population of expanded natural killer cells is the same or higher than the percentage of natural killer cells in the seed cells from umbilical cord blood.

In some embodiments, the percentage of NK cells expressing NKG2D in the population of expanded natural killer cells is the same or higher than the percentage of natural killer cells in the seed cells from umbilical cord blood.

In some embodiments, the percentage of NK cells expressing NKp30 in the population of expanded natural killer cells is the same or higher than the percentage of natural killer cells in the seed cells from umbilical cord blood.

In some embodiments, the percentage of NK cells expressing NKp44 in the population of expanded natural killer cells is the same or higher than the percentage of natural killer cells in the seed cells from umbilical cord blood.

In some embodiments, the percentage of NK cells expressing NKp46 in the population of expanded natural killer cells is the same or higher than the percentage of natural killer cells in the seed cells from umbilical cord blood.

In some embodiments, the percentage of NK cells expressing DNAM-1 in the population of expanded natural killer cells is the same or higher than the percentage of natural killer cells in the seed cells from umbilical cord blood.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative and are not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a method for NK cell expansion and stimulation.

FIG. 2 shows that cord blood-derived NK cells (CB-NK) have an approximately ten-fold greater ability to expand in culture than peripheral blood-derived NK cells (PB-NK) in preclinical studies.

FIG. 3 shows that expression of tumor-engaging NK activating immune receptors was higher and more consistent in cord blood-derived drug product compared to that generated from peripheral blood.

FIG. 4 shows phenotypes of expanded and stimulated population of NK cells.

FIG. 5 shows key steps in the manufacture of the AB-101 drug product, which is an example of a cord blood-derived and expanded population of NK cells.

FIG. 6 shows the purity of AB-101 (n=9).

FIG. 7 shows purity of CD3 depleted cells, MCB and DP manufactured in GMP conditions.

FIG. 8 shows expression of NK cell receptors on CD3 depleted cells, MCB and DP manufactured in GMP conditions.

FIG. 9 shows NK purity (CD56+/CD3−) by flow cytometry.

FIG. 10 shows CD38+ expression of expanded NK cells from three different cord blood donors.

FIG. 11 shows CD38+ mean fluorescence intensity of CD38+NK cells from three different cord blood donors.

FIG. 12 shows differential surface protein expression of starting NK cell source compared to AB-101 cells.

FIG. 13 shows that AB-101 in combination with the anti-HER2 monoclonal antibody trastuzumab resulted in substantial cytotoxic activity against the HER2+ cell line NCI-N87. Effector-to-target ratio (E:T 1:1).

FIG. 14 shows in vitro characterization of AB-101+ Trastuzumab.

FIG. 15 shows in vivo characterization of AB-101+ Trastuzumab.

FIG. 16 shows in vivo characterization of AB-101+ Trastuzumab.

### DETAILED DESCRIPTION

Provided herein are, amongst other things, Natural Killer (NK) cells, e.g., expanded and stimulated NK cells, methods for producing the NK cells, pharmaceutical compositions comprising the NK cells, and methods of treating patients suffering, e.g., from cancer, with the NK cells.

### I. Expansion and Stimulation of Natural Killer Cells

In some embodiments, natural killer cells are expanded and stimulated, e.g., by culturing and stimulation with feeder cells.

NK cells can be expanded and stimulated as described, for example, in US 2020/0108096 or WO 2020/101361, both of which are incorporated herein by reference in their entirety. Briefly, the source cells can be cultured on modified HuT-78 (ATCC® TIB-161™) cells that have been engineered to express 4-1BBL, membrane bound IL-21, and a mutant TNFα as described in US 2020/0108096.

Suitable NK cells can also be expanded and stimulated as described herein.

In some embodiments, NK cells are expanded and stimulated by a method comprising: (a) providing NK cells, e.g., a composition comprising NK cells, e.g., CD3(−) depleted cells; and (b) culturing in a medium comprising feeder cells and/or stimulation factors, thereby producing a population of expanded and stimulated NK cells.

### A. Natural Killer Cell Sources

In some embodiments, the NK cell source is selected from the group consisting of peripheral blood, peripheral blood lymphocytes (PBLs), peripheral blood mononuclear cells (PBMCs), bone marrow, umbilical cord blood (cord blood), isolated NK cells, NK cells derived from induced pluripotent stem cells, NK cells derived from embryonic stem cells, and combinations thereof.

In some embodiments, the NK cell source is a single unit of cord blood.

In some embodiments, the natural killer cell source, e.g., single unit of cord blood, comprises from or from about 1×10⁷ to or to about 1×10⁹ total nucleated cells. In some embodiments, the natural killer cell source, e.g., single unit of cord blood, comprises from or from about 1×10⁸ to or to about 1.5×10⁸ total nucleated cells. In some embodiments, the natural killer cell source, e.g., single unit of cord blood, comprises 1×10⁸ total nucleated cells. In some embodiments, the natural killer cell source, e.g., single unit of cord blood, comprises about 1×10⁸ total nucleated cells. In some embodiments, the natural killer cell source, e.g., single unit of cord blood, comprises 1×10⁹ total nucleated cells. In some embodiments, the natural killer cell source, e.g., single unit of cord blood, comprises about 1×10⁹ total nucleated cells.

In some embodiments, the NK cell source, e.g., the cord blood unit, comprises from about 20% to about 80% CD16+ cells. In some embodiments, the NK cell source, e.g., the cord blood unit, comprises from or from about 20% to or to about 80%, from about 20% to or to about 70%, from about 20% to or to about 60%, from about 20% to or to about 50%, from about 20% to or to about 40%, from about 20% to or to about 30%, from about 30% to or to about 80%, from about 30% to or to about 70%, from about 30% to or to about 60%, from about 30% to or to about 50%, from about 30% to or to about 40%, from about 40% to or to about 80%, from about 40% to or to about 70%, from about 40% to or to about 60%, from about 40% to or to about 50%, from about 50% to or to about 80%, from about 50% to or to about 70%, from about 50% to or to about 60%, from about 60% to or to about 80%, from about 60% to or to about 70%, or from about 70% to or to about 80% CD16+ cells. In some embodiments, the NK cell source, e.g., the cord blood unit, comprises less than or equal to 80% CD16+ cells. Alternately, some NK cell sources may comprise CD16+ cells at a concentration of greater than 80%.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% MLG2A+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2C+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2D+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp46+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp30+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% DNAM-1+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp44+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD25+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD62L+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD69+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CXCR3+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD57+ cells.

In some embodiments, NK cells in the NK cell source comprise a KIR B allele of the KIR receptor family. See, e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5:e15115 (2010).

In some embodiments, NK cells in the NK cell source comprise the 158 V/V variant of CD16 (i.e. homozygous CD16 158V polymorphism). See, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).

In some embodiments, NK cells in the cell source comprises both the KIR B allele of the KIR receptor family and the 158 V/V variant of CD16.

In some embodiments, the NK cells in the cell source are not genetically engineered.

In some embodiments, the NK cells in the cell source do not comprise a CD16 transgene.

In some embodiments, the NK cells in the cell source do not express an exogenous CD16 protein.

In some embodiments, the NK cell source is CD3(+) depleted. In some embodiments, the method comprises depleting the NK cell source of CD3(+) cells. In some embodiments, depleting the NK cell source of CD3(+) cells comprises contacting the NK cell source with a CD3 binding antibody or antigen binding fragment thereof. In some embodiments, the CD3 binding antibody or antigen binding fragment thereof is selected from the group consisting of OKT3, UCHT1, and HIT3a, and fragments thereof. In some embodiments, the CD3 binding antibody or antigen binding fragment thereof is OKT3 or an antigen binding fragment thereof. In some embodiments, the antibody or antigen binding fragment thereof is attached to a bead, e.g., a magnetic bead. In some embodiments, the depleting the composition of CD3(+) cells comprises contacting the composition with a CD3 targeting antibody or antigen binding fragment thereof attached to a bead and removing the bead-bound CD3(+) cells from the composition. The composition can be depleted of CD3 cells by immunomagnetic selection, for example, using a CliniMACS T cell depletion set ((LS Depletion set (162-01) Miltenyi Biotec).

In some embodiments, the NK cell source CD56+ enriched, e.g., by gating on CD56 expression.

In some embodiments, the NK cell source is both CD56+ enriched and CD3(+) depleted, e.g., by selecting for cells with CD56+CD3− expression.

In some embodiments, the NK cell source comprises both the KIR B allele of the KIR receptor family and the 158 V/V variant of CD16 and is + enriched and CD3(+) depleted, e.g., by selecting for cells with CD56+CD3− expression.

### B. Feeder Cells

Disclosed herein are feeder cells for the expansion of NK cells. These feeder cells advantageously allow NK cells to expand to numbers suitable for the preparation of a pharmaceutical composition as discussed herein. In some cases, the feeder cells allow the expansion of NK cells without the loss of CD16 expression, which often accompanies cell expansion on other types of feeder cells or using other methods. In some cases, the feeder cells make the expanded NK cells more permissive to freezing such that a higher proportion of NK cells remain viable after a freeze/thaw cycle or such that the cells remain viable for longer periods of time while frozen. In some cases, the feeder cells allow the NK cells to retain high levels of cytotoxicity, including ADCC, extend survival, increase persistence, and enhance or retain high levels of CD16. In some cases, the feeder cells allow the NK cells to expand without causing significant levels of exhaustion or senescence.

Feeder cells can be used to stimulate the NK cells and help them to expand more quickly, e.g., by providing substrate, growth factors, and/or cytokines.

NK cells can be stimulated using various types of feeder cells, including, but not limited to peripheral blood mononuclear cells (PBMC), Epstein-Barr virus-transformed B-lymphoblastoid cells (e.g., EBV-LCL), myelogenous leukemia cells (e.g., K562), and CD4(+) T cells (e.g., HuT), and derivatives thereof.

In some embodiments, the feeder cells are inactivated, e.g., by γ-irradiation or mitomycin-c treatment.

Suitable feeder cells for use in the methods described herein are described, for example, in US 2020/0108096, which is hereby incorporated by reference in its entirety.

In some embodiments, the feeder cell(s) are inactivated CD4(+) T cell(s). In some embodiments, the inactivated CD4(+) T cell(s) are HuT-78 cells (ATCC® TIB-161™) or variants or derivatives thereof. In some embodiments, the HuT-78 derivative is H9 (ATCC® HTB-176™).

In some embodiments, the inactivated CD4(+) T cell(s) express OX40L. In some embodiments, the inactivated CD4(+) T cell(s) are HuT-78 cells or variants or derivatives thereof that express OX40L SEQ ID NO: 4) or a variant thereof.

In some embodiments, the feeder cells are HuT-78 cells engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

In some embodiments, the inactivated CD4(+) T cell(s) are HuT-78 (ATCC® TIB-161™) cells or variants or derivatives thereof that express an ortholog of OX40L, or variant thereof. In some embodiments, the feeder cells are HuT-78 cells engineered to express at least one gene selected from the group consisting of an 4-1BBL ortholog or variant thereof, a membrane bound IL-21 ortholog or variant thereof, and mutant TNFalpha ortholog, or variant thereof.

In some embodiments, the feeder cells are HuT-78 cell(s) that express OX40L SEQ ID NO: 4) and are engineered to express 4-1BBL (SEQ ID NO: 1), membrane bound IL-21 (SEQ ID NO: 2), and mutant TNFalpha (SEQ ID NO: 3) (“eHut-78 cells”) or variants or derivatives thereof.

In some embodiments, the feeder cells are expanded, e.g., from a frozen stock, before culturing with NK cells, e.g., as described in Example 2.

### C. Stimulating Factors

NK cells can also be stimulated using one or more stimulation factors other than feeder cells, e.g., signaling factors, in addition to or in place of feeder cells.

In some embodiments, the stimulating factor, e.g., signaling factor, is a component of the culture medium, as described herein. In some embodiments, the stimulating factor, e.g., signaling factor, is a supplement to the culture medium, as described herein.

In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof.

In some embodiments, the cytokine is IL-2.

In some embodiments, the cytokines are a combination of IL-2 and IL-15.

In some embodiments, the cytokines are a combination of IL-2, IL-15, and IL-18.

In some embodiments, the cytokines are a combination of IL-2, IL-18, and IL-21.

### D. Culturing

The NK cells can be expanded and stimulated by co-culturing an NK cell source and feeder cells and/or other stimulation factors. Suitable NK cell sources, feeder cells, and stimulation factors are described herein.

In some cases, the resulting population of expanded natural killer cells is enriched and/or sorted after expansion. In some cases, the resulting population of expanded natural killer cells is not enriched and/or sorted after expansion

Also described herein are compositions comprising the various culture compositions described herein, e.g., comprising NK cells. For example, a composition comprising a population of expanded cord blood-derived natural killer cells comprising a KIR-B haplotype and homozygous for a CD16 158V polymorphism and a plurality of engineered HuT78 cells.

Also described herein are vessels, e.g., vials, cryobags, and the like, comprising the resulting populations of expanded natural killer cells. In some cases, a plurality of vessels comprising portions of the resulting populations of expanded natural killer cells, e.g., at least 10, e.g., 20, 30, 40, 50, 60, 70, 80, 90, 100, 125, 150, 175, 200, 250, 300, 400, 500, 600, 700, 800, 900, 1000, 1100, or 1200 vessels.

### 1. Culture Medium

Disclosed herein are culture media for the expansion of NK cells. These culture media advantageously allow NK cells to expand to numbers suitable for the preparation of a pharmaceutical composition as discussed herein. In some cases, the culture media allows NK cells to expand without the loss of CD16 expression that often accompanies cell expansion on other helper cells or in other media.

In some embodiments, the culture medium is a basal culture medium, optionally supplemented with additional components, e.g., as described herein.

In some embodiments, the culture medium, e.g., the basal culture medium, is a serum-free culture medium. In some embodiments, the culture medium, e.g., the basal culture medium, is a serum-free culture medium supplemented with human plasma and/or serum.

Suitable basal culture media include, but are not limited to, DMEM, RPMI 1640, MEM, DMEM/F12, SCGM (CellGenix®, 20802-0500 or 20806-0500), LGM-3™ (Lonza, CC-3211), TexMACS™ (Miltenyi Biotec, 130-097-196), ALyS™ 505NK-AC (Cell Science and Technology Institute, Inc., 01600P02), ALyS™ 505NK-EX (Cell Science and Technology Institute, Inc., 01400P10), CTS™ AIM-V™ SFM (ThermoFisher Scientific, A3830801), CTS™ OpTmizer™ (ThermoFisher Scientific, A1048501, ABS-001, StemXxVivoand combinations thereof.

The culture medium may comprise additional components, or be supplemented with additional components, such as growth factors, signaling factors, nutrients, antigen binders, and the like. Supplementation of the culture medium may occur by adding each of the additional component or components to the culture vessel either before, concurrently with, or after the medium is added to the culture vessel. The additional component or components may be added together or separately. When added separately, the additional components need not be added at the same time.

In some embodiments, the culture medium comprises plasma, e.g., human plasma. In some embodiments, the culture medium is supplemented with plasma, e.g., human plasma. In some embodiments, the plasma, e.g., human plasma, comprises an anticoagulant, e.g., trisodium citrate.

In some embodiments, the medium comprises and/or is supplemented with from or from about 0.5% to or to about 10% v/v plasma, e.g., human plasma. In some embodiments, the medium is supplemented with from or from about 0.5% to or to about 9%, from or from about 0.5% to or to about 8%, from or from about 0.5% to or to about 7%, from or from about 0.5% to or to about 6%, from or from about 0.5% to or to about 5%, from or from about 0.5% to or to about 4%, from or from about 0.5% to or to about 3%, from or from about 0.5% to or to about 2%, from or from about 0.5% to or to about 10%, from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 2% to or to about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 4% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v plasma, e.g., human plasma. In some embodiments, the culture medium comprises and/or is supplemented with from 0.8% to 1.2% v/v human plasma. In some embodiments, the culture medium comprises and/or is supplemented with 1.0% v/v human plasma. In some embodiments, the culture medium comprises and/or is supplemented with about 1.0% v/v human plasma.

In some embodiments, the culture medium comprises serum, e.g., human serum. In some embodiments, the culture medium is supplemented with serum, e.g., human serum. In some embodiments, the serum is inactivated, e.g., heat inactivated. In some embodiments, the serum is filtered, e.g., sterile-filtered.

In some embodiments, the culture medium comprises glutamine. In some embodiments, the culture medium is supplemented with glutamine. In some embodiments, the culture medium comprises and/or is supplemented with from or from about 2.0 to or to about 6.0 mM glutamine. In some embodiments, the culture medium comprises and/or is supplemented with from or from about 2.0 to or to about 5.5, from or from about 2.0 to or to about 5.0, from or from about 2.0 to or to about 4.5, from or from about 2.0 to or to about 4.0, from or from about 2.0 to or to about 3.5, from or from about 2.0 to or to about 3.0, from or from about 2.0 to or to about 2.5, from or from about 2.5 to or to about 6.0, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3.0 to or to about 6.0, from or from about 3.0 to or to about 5.5, from or from about 3.0 to or to about 5.0, from or from about 3.0 to or to about 4.5, from or from about 3.0 to or to about 4.0, from or from about 3.0 to or to about 3.5, from or from about 3.5 to or to about 6.0, from or from about 3.5 to or to about 5.5, from or from about 3.5 to or to about 5.0, from or from about 3.5 to or to about 4.5, from or from about 3.5 to or to about 4.0, from or from about 4.0 to or to about 6.0, from or from about 4.0 to or to about 5.5, from or from about 4.0 to or to about 5.0, from or from about 4.0 to or to about 4.5, from or from about 4.5 to or to about 6.0, from or from about 4.5 to or to about 5.5, from or from about 4.5 to or to about 5.0, from or from about 5.0 to or to about 6.0, from or from about 5.0 to or to about 5.5, or from or from about 5.5 to or to about 6.0 mM glutamine. In some embodiments, the culture medium comprises and/or is supplemented with from 3.2 mM glutamine to 4.8 mM glutamine. In some embodiments, the culture medium comprises and/or is supplemented with 4.0 mM glutamine. In some embodiments, the culture medium comprises and/or is supplemented with about 4.0 mM glutamine.

In some embodiments, the culture medium comprises one or more cyotkines. In some embodiments, the culture medium is supplemented with one or more cyotkines.

In some embodiments, the cytokine is selected from IL-2, IL-12, IL-15, IL-18, and combinations thereof.

In some embodiments, the culture medium comprises and/or is supplemented with IL-2. In some embodiments, the culture medium comprises and/or is supplemented with from or from about 150 to or to about 2,500 IU/mL IL-2. In some embodiments, the culture medium comprises and/or is supplemented with from or from about 200 to or to about 2,250, from or from about 200 to or to about 2,000, from or from about 200 to or to about 1,750, from or from about 200 to or to about 1,500, from or from about 200 to or to about 1,250, from or from 200 to or to about 1,000, from or from about 200 to or to about 750, from or from about 200 to or to about 500, from or from about 200 to or to about 250, from or from about 250 to or to about 2,500, from or from about 250 to or to about 2,250, from or from about 250 to or to about 2,000, from or from about 250 to or to about 1,750, from or from about 250 to or to about 1,500, from or from about 250 to or to about 1,250, from or from about 250 to or to about 1,000, from or from about 250 to or to about 750, from or from about 250 to or to about 500, from or from about 500 to or to about 2,500, from or from about 500 to or to about 2,250, from or from about 500 to or to about 2,000, from or from about 500 to or to about 1,750, from or from about 500 to or to about 1,500, from or from about 500 to or to about 1,250, from or from about 500 to or to about 1,000, from or from about 500 to or to about 750, from or from about 750 to or to about 2,250, from or from about 750 to or to about 2,000, from or from about 750 to or to about 1,750, from or from about 750 to or to about 1,500, from or from about 750 to or to about 1,250, from or from about 750 to or to about 1,000, from or from about 1,000 to or to about 2,500, from or from about 1,000 to or to about 2,250, from or from about 1,000 to or to about 2,000, from or from about 1,000 to or to about 1,750, from or from about 1,000 to or to about 1,500, from or from about 1,000 to or to about 1,250, from or from about 1,250 to or to about 2,500, from or from about 1,250 to or to about 2,250, from or from about 1,250 to or to about 2,000, from or from about 1,250 to or to about 1,750, from or from about 1,250 to or to about 1,500, from or from about 1,500 to or to about 2,500, from or from about 1,500 to or to about 2,250, from or from about 1,500 to or to about 2,000, from or from about 1,500 to or to about 1,750, from or from about 1,750 to or to about 2,500, from or from about 1,750 to or to about 2,250, from or from about 1,750 to or to about 2,000, from or from about 2,000 to or to about 2,500, from or from about 2,000 to or to about 2,250, or from or from about 2,250 to or to about 2,500 IU/mL IL-2.

In some embodiments, the culture medium comprises and/or is supplemented with from 64 μg/L to 96 μg/L IL-2. In some embodiments, the culture medium comprises and/or is supplemented with 80 μg/L IL-2 (approximately 1,333 IU/mL). In some embodiments, the culture medium comprises and/or is supplemented with about 80 μg/L.

In some embodiments, the culture medium comprises and/or is supplemented with a combination of IL-2 and IL-15.

In some embodiments, the culture medium comprises and/or is supplemented with a combination of IL-2, IL-15, and IL-18.

In some embodiments, the culture medium comprises and/or is supplemented with a combination of IL-2, IL-18, and IL-21.

In some embodiments, the culture medium comprises and/or is supplemented with glucose. In some embodiments, the culture medium comprises and/or is supplemented with from or from about 0.5 to or to about 3.5 g/L glucose. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L glucose. In some embodiments, the culture medium comprises and/or is supplemented with from 1.6 to 2.4 g/L glucose. In some embodiments, the culture medium comprises and/or is supplemented with 2.0 g/L glucose. In some embodiments, the culture medium comprises about 2.0 g/L glucose.

In some embodiments, the culture medium comprises and/or is supplemented with sodium pyruvate. In some embodiments, the culture medium comprises and/or is supplemented with from or from about 0.1 to or to about 2.0 mM sodium pyruvate. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 mM sodium pyruvate. In some embodiments, the culture medium comprises from 0.8 to 1.2 mM sodium pyruvate. In some embodiments, the culture medium comprises 1.0 mM sodium pyruvate. In some embodiments, the culture medium comprises about 1.0 mM sodium pyuruvate.

In some embodiments, the culture medium comprises and/or is supplemented with sodium hydrogen carbonate. In some embodiments, the culture medium comprises and/or is supplemented with from or from about 0.5 to or to about 3.5 g/L sodium hydrogen carbonate. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L sodium hydrogen carbonate. In some embodiments, the culture medium comprises and/or is supplemented with from 1.6 to 2.4 g/L sodium hydrogen carbonate. In some embodiments, the culture medium comprises and/or is supplemented with 2.0 g/L sodium hydrogen carbonate. In some embodiments, the culture medium comprises about 2.0 g/L sodium hydrogen carbonate.

In some embodiments, the culture medium comprises and/or is supplemented with albumin, e.g., human albumin, e.g., a human albumin solution described herein. In some embodiments, the culture medium comprises and/or is supplemented with from or from about 0.5% to or to about 3.5% v/v of a 20% albumin solution, e.g., a 20% human albumin solution. In some embodiments, the culture medium comprises and/or is supplemented with from or from about 0.5% to or to about 3.0%, from or from about 0.5% to or to about 2.5%, from or from about 0.5% to or to about 2.0%, from or from about 0.5% to or to about 1.5%, from or from about 0.5% to or to about 1.0%, from or from about 1.0% to or to about 3.0%, from or from about 1.0% to or to about 2.5%, from or from about 1.0% to or to about 2.0%, from or from about 1.0% to or to about 1.5%, from or from about 1.5% to or to about 3.0%, from or from about 1.5% to or to about 2.5%, from or from about 1.5% to or to about 2.0%, from or from about 2.0% to or to about 3.0%, from or from about 2.0% to or to about 2.5%, or from or from about 2.5% to or to about 3.0% v/v of a 20% albumin solution, e.g., a 20% human albumin solution. In some embodiments, the culture medium comprises and/or is supplemented with from 1.6% to 2.4% v/v of a 20% albumin solution, e.g., a 20% human albumin solution. In some embodiments, the culture medium comprises and/or is supplemented with 2.0% v/v of a 20% albumin solution, e.g., a 20% human albumin solution. In some embodiments, the culture medium comprises about 2.0% v/v of a 20% albumin solution, e.g., a 20% human albumin solution.

In some embodiments, the culture medium comprises and/or is supplemented with from or from about 2 to or to about 6 g/L albumin, e.g., human albumin. In some embodiments, the culture medium comprises and/or is supplemented with from or from about 2 to or to about 5.5, from or from about 2 to or to about 5.0, from or from about 2 to or to about 4.5, from or from about 2 to or to about 4, from or from about 2 to or to about 3.5, from or from about 2 to or to about 3, from or from about 2 to or to about 2.5, from or from about 2.5 to or to about 6, from or from about 2.5 to or to about 5.5, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3 to or to about 6, from or from about 3 to or to about 5.5, from or from about 3 to or to about 5, from or from about 3 to or to about 4.5, from or from about 3 to or to about 4, from or from about 3 to or to about 3.5, from or from about 3.5 to or to about 6, from or from about 3.5 to or to about 5.5, from or from about 3.5 to or to about 5, from or from about 3.5 to or to about 4.5, from or from about 3.5 to or to about 4, from or from about 4 to or to about 6, from or from about 4 to or to about 5.5, from or from about 4 to or to about 5, from or from about 4 to or to about 4.5, from or from about 4.5 to or to about 6, from or from about 4.5 to or to about 5.5, from or from about 4.5 to or to about 5, from or from about 5 to or to about 6, from or from about 5 to or to about 5.5, or from or from about 5.5 to or to about 6 g/L albumin, e.g., human albumin. In some embodiments, the culture medium comprises and/or is supplemented with from 3.2 to 4.8 g/L albumin, e.g., human albumin. In some embodiments, the culture medium comprises 4 g/L albumin, e.g., human albumin. In some embodiments, the culture medium comprises about 4 g/L albumin, e.g., human albumin

In some embodiments, the culture medium is supplemented with Poloxamer 188. In some embodiments, the culture medium comprises and/or is supplemented with from or from about 0.1 to or to about 2.0 g/L Poloxamer 188. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 g/L Poloxamer 188. In some embodiments, the culture medium comprises from 0.8 to 1.2 g/L Poloxamer 188. In some embodiments, the culture medium comprises 1.0 g/L Poloxamer 188. In some embodiments, the culture medium comprises about 1.0 g/L Poloxamer 188.

In some embodiments, the culture medium comprises and/or is supplemented with one or more antibiotics.

A first exemplary culture medium is set forth in Table 1.

TABLE 1
Exemplary Culture Medium #1

| | Exemplary Concentration | | Exemplary | |
|---|---|---|---|---|
| Component | Range | | Concentration | |
| CellgroSCGM liquid | undiluted | | undiluted | |
| medium | | | | |
| Human Plasma | 0.8-1.2% | (v/v) | 1.0% | v/v |
| Glutamine | 3.2-4.8 | mM | 4.0 | mM |
| IL-2 | 64-96 | μg/L | 80 | μg/L |

A second exemplary culture medium is set forth in Table 2.

TABLE 2
Exemplary Culture Medium #2

| | Exemplary | | Exemplary | |
|---|---|---|---|---|
| Component | Concentration Range | | Concentration | |
| RPMI1640 | 7.6-13.2 | g/L | 10.4 | g/L |
| Human Plasma | 0.8-1.2% | (v/v) | 1.0% | v/v |
| Glucose | 1.6-2.4 | g/L | 2.0 | g/L |
| Glutamine | 3.2-4.8 | mM | 4.0 | mM |
| Sodium Pyruvate | 0.8-1.2 | mM | 1.0 | mM |
| Sodium Hydrogen Carbonate | 1.6-2.4 | g/L | 2.0 | g/L |
| IL-2 | 64-96 | μg/L | 80 | μg/L |
| Albumin 20% solution | 1.6-2.5% v/v | | 2.0% v/v | |
| | (3.2 to 4.8 g/L) | | (4.0 g/L) | |
| Poloxamer 188 | 0.8-1.2 | g/L | 1.0 | g/L |

### 2. CD3 Binding Antibodies

In some embodiments, the culture medium comprises and/or is supplemented with a CD3 binding antibody or antigen binding fragment thereof. In some embodiments, the CD3 binding antibody or antigen binding fragment thereof is selected from the group consisting of OKT3, UCHT1, and HIT3a, or variants thereof. In some embodiments, the CD3 binding antibody or antigen binding fragment thereof is OKT3 or an antigen binding fragment thereof.

In some embodiments, the CD3 binding antibody or antigen binding fragment thereof and feeder cells are added to the culture vessel before addition of NK cells and/or culture medium.

In some embodiments, the culture medium comprises and/or is supplemented with from or from about 5 ng/mL to or to about 15 ng/mL OKT3. In some embodiments, the culture medium comprises and/or is supplemented with from or from about 5 to or to about 12.5, from or from about 5 to or to about 10, from or from about 5 to or to about 7.5, from or from about 7.5 to or to about 15, from or from about 7.5 to or to about 12.5, from or from about 7.5 to or to about 10, from or from about 10 to or to about 15, from or from about 10 to or to about 12.5, or from or from about 12.5 to or to about 15 ng/mL OKT3. In some embodiments, the culture medium comprises and/or is supplemented with 10 ng/mL OKT3. In some embodiments, the culture medium comprises and/or is supplemented with about 10 ng/mL OKT3.

### 3. Culture Vessels

A number of vessels are consistent with the disclosure herein. In some embodiments, the culture vessel is selected from the group consisting of a flask, a bottle, a dish, a multiwall plate, a roller bottle, a bag, and a bioreactor.

In some embodiments, the culture vessel is treated to render it hydrophilic. In some embodiments, the culture vessel is treated to promote attachment and/or proliferation. In some embodiments, the culture vessel surface is coated with serum, collagen, laminin, gelatin, poly-L-lysine, fibronectin, extracellular matrix proteins, and combinations thereof.

In some embodiments, different types of culture vessels are used for different stages of culturing.

In some embodiments, the culture vessel has a volume of from or from about 100 mL to or to about 1,000 L. In some embodiments, the culture vessel has a volume of or about 125 mL, of or about 250 mL, of or about 500 mL, of or about 1 L, of or about 5 L, of about 10 L, or of or about 20 L.

In some embodiments, the culture vessel is a bioreactor.

In some embodiments, the bioreactor is a rocking bed (wave motion) bioreactor. In some embodiments, the bioreactor is a stirred tank bioreactor. In some embodiments, the bioreactor is a rotating wall vessel. In some embodiments, the bioreactor is a perfusion bioreactor. In some embodiments, the bioreactor is an isolation/expansion automated system. In some embodiments, the bioreactor is an automated or semi-automated bioreactor. In some embodiments, the bioreactor is a disposable bag bioreactor.

In some embodiments, the bioreactor has a volume of from about 100 mL to about 1,000 L. In some embodiments, the bioreactor has a volume of from about 10 L to about 1,000 L. In some embodiments, the bioreactor has a volume of from about 100 L to about 900 L. In some embodiments, the bioreactor has a volume of from about 10 L to about 800 L. In some embodiments, the bioreactor has a volume of from about 10 L to about 700 L, about 10 L to about 600 L, about 10 L to about 500 L, about 10 L to about 400 L, about 10 L to about 300 L, about 10 L to about 200 L, about 10 L to about 100 L, about 10 L to about 90 L, about 10 L to about 80 L, about 10 L to about 70 L, about 10 L to about 60 L, about 10 L to about 50 L, about 10 L to about 40 L, about 10 L to about 30 L, about 10 L to about 20 L, about 20 L to about 1,000 L, about 20 L to about 900 L, about 20 L to about 800 L, about 20 L to about 700 L, about 20 L to about 600 L, about 20 L to about 500 L, about 20 L to about 400 L, about 20 L to about 300 L, about 20 L to about 200 L, about 20 L to about 100 L, about 20 L to about 90 L, about 20 L to about 80 L, about 20 L to about 70 L, about 20 L to about 60 L, about 20 L to about 50 L, about 20 L to about 40 L, about 20 L to about 30 L, about 30 L to about 1,000 L, about 30 L to about 900 L, about 30 L to about 800 L, about 30 L to about 700 L, about 30 L to about 600 L, about 30 L to about 500 L, about 30 L to about 400 L, about 30 L to about 300 L, about 30 L to about 200 L, about 30 L to about 100 L, about 30 L to about 90 L, about 30 L to about 80 L, about 30 L to about 70 L, about 30 L to about 60 L, about 30 L to about 50 L, about 30 L to about 40 L, about 40 L to about 1,000 L, about 40 L to about 900 L, about 40 L to about 800 L, about 40 L to about 700 L, about 40 L to about 600 L, about 40 L to about 500 L, about 40 L to about 400 L, about 40 L to about 300 L, about 40 L to about 200 L, about 40 L to about 100 L, about 40 L to about 90 L, about 40 L to about 80 L, about 40 L to about 70 L, about 40 L to about 60 L, about 40 L to about 50 L, about 50 L to about 1,000 L, about 50 L to about 900 L, about 50 L to about 800 L, about 50 L to about 700 L, about 50 L to about 600 L, about 50 L to about 500 L, about 50 L to about 400 L, about 50 L to about 300 L, about 50 L to about 200 L, about 50 L to about 100 L, about 50 L to about 90 L, about 50 L to about 80 L, about 50 L to about 70 L, about 50 L to about 60 L, about 60 L to about 1,000 L, about 60 L to about 900 L, about 60 L to about 800 L, about 60 L to about 700 L, about 60 L to about 600 L, about 60 L to about 500 L, about 60 L to about 400 L, about 60 L to about 300 L, about 60 L to about 200 L, about 60 L to about 100L, about 60 L to about 90 L, about 60 L to about 80 L, about 60 L to about 70 L, about 70 L to about 1,000 L, about 70 L to about 900 L, about 70 L to about 800 L, about 70 L to about 700 L, about 70 L to about 600 L, about 70 L to about 500 L, about 70 L to about 400 L, about 70 L to about 300 L, about 70 L to about 200 L, about 70 L to about 100 L, about 70 L to about 90 L, about 70 L to about 80 L, about 80 L to about 1,000 L, about 80 L to about 900 L, about 80 L to about 800 L, about 80 L to about 700 L, about 80 L to about 600 L, about 80 L to about 500 L, about 80 L to about 400 L, about 80 L to about 300 L, about 80 L to about 200 L, about 80 L to about 100 L, about 80 L to about 90 L, about 90 L to about 1,000 L, about 90 L to about 900 L, about 90 L to about 800 L, about 90 L to about 700 L, about 90 L to about 600 L, about 90 L to about 500 L, about 90 L to about 400 L, about 90 L to about 300 L, about 90 L to about 200 L, about 90 L to about 100 L, about 100 L to about 1,000 L, about 100 L to about 900 L, about 100 L to about 800 L, about 100 L to about 700 L, about 100 L to about 600 L, about 100 L to about 500 L, about 100 L to about 400 L, about 100 L to about 300 L, about 100 L to about 200 L, about 200 L to about 1,000 L, about 200 L to about 900 L, about 200 L to about 800 L, about 200 L to about 700 L, about 200 L to about 600 L, about 200 L to about 500 L, about 200 L to about 400 L, about 200 L to about 300 L, about 300 L to about 1,000 L, about 300 L to about 900 L, about 300 L to about 800 L, about 300 L to about 700 L, about 300 L to about 600 L, about 300 L to about 500 L, about 300 L to about 400 L, about 400 L to about 1,000 L, about 400 L to about 900 L, about 400 L to about 800 L, about 400 L to about 700 L, about 400 L to about 600 L, about 400 L to about 500 L, about 500 L to about 1,000 L, about 500 L to about 900 L, about 500 L to about 800 L, about 500 L to about 700 L, about 500 L to about 600 L, about 600 L to about 1,000 L, about 600 L to about 900 L, about 600 L to about 800 L, about 600 L to about 700 L, about 700 L to about 1,000 L, about 700 L to about 900 L, about 700 L to about 800 L, about 800 L to about 1,000 L, about 800 L to about 900 L, or about 900 L to about 1,000 L. In some embodiments, the bioreactor has a volume of about 50 L.

In some embodiments, the bioreactor has a volume of from 100 mL to 1,000 L. In some embodiments, the bioreactor has a volume of from 10 L to 1,000 L. In some embodiments, the bioreactor has a volume of from 100 L to 900 L. In some embodiments, the bioreactor has a volume of from 10 L to 800 L. In some embodiments, the bioreactor has a volume of from 10 L to 700 L, 10 L to 600 L, 10 L to 500 L, 10 L to 400 L, 10 L to 300 L, 10 L to 200 L, 10 L to 100 L, 10 L to 90 L, 10 L to 80 L, 10 L to 70 L, 10 L to 60 L, 10 L to 50 L, 10 L to 40 L, 10 L to 30 L, 10 L to 20 L, 20 L to 1,000 L, 20 L to 900 L, 20 L to 800 L, 20 L to 700 L, 20 L to 600 L, 20 L to 500 L, 20 L to 400 L, 20 L to 300 L, 20 L to 200 L, 20 L to 100 L, 20 L to 90 L, 20 L to 80 L, 20 L to 70 L, 20 L to 60 L, 20 L to 50 L, 20 L to 40 L, 20 L to 30 L, 30 L to 1,000 L, 30 L to 900 L, 30 L to 800 L, 30 L to 700 L, 30 L to 600 L, 30 L to 500 L, 30 L to 400 L, 30 L to 300 L, 30 L to 200 L, 30 L to 100 L, 30 L to 90 L, 30 L to 80 L, 30 L to 70 L, 30 L to 60 L, 30 L to 50 L, 30 L to 40 L, 40 L to 1,000 L, 40 L to 900 L, 40 L to 800 L, 40 L to 700 L, 40 L to 600 L, 40 L to 500 L, 40 L to 400 L, 40 L to 300 L, 40 L to 200 L, 40 L to 100 L, 40 L to 90 L, 40 L to 80 L, 40 L to 70 L, 40 L to 60 L, 40 L to 50 L, 50 L to 1,000 L, 50 L to 900 L, 50 L to 800 L, 50 L to 700 L, 50 L to 600 L, 50 L to 500 L, 50 L to 400 L, 50 L to 300 L, 50 L to 200 L, 50 L to 100 L, 50 L to 90 L, 50 L to 80 L, 50 L to 70 L, 50 L to 60 L, 60 L to 1,000 L, 60 L to 900 L, 60 L to 800 L, 60 L to 700 L, 60 L to 600 L, 60 L to 500 L, 60 L to 400 L, 60 L to 300 L, 60 L to 200 L, 60 L to 100 L, 60 L to 90 L, 60 L to 80 L, 60 L to 70 L, 70 L to 1,000 L, 70 L to 900 L, 70 L to 800 L, 70 L to 700 L, 70 L to 600 L, 70 L to 500 L, 70 L to 400 L, 70 L to 300 L, 70 L to 200 L, 70 L to 100 L, 70 L to 90 L, 70 L to 80 L, 80 L to 1,000 L, 80 L to 900 L, 80 L to 800 L, 80 L to 700 L, 80 L to 600 L, 80 L to 500 L, 80 L to 400 L, 80 L to 300 L, 80 L to 200 L, 80 L to 100 L, 80 L to 90 L, 90 L to 1,000 L, 90 L to 900 L, 90 L to 800 L, 90 L to 700 L, 90 L to 600 L, 90 L to 500 L, 90 L to 400 L, 90 L to 300 L, 90 L to 200 L, 90 L to 100 L, 100 L to 1,000 L, 100 L to 900 L, 100 L to 800 L, 100 L to 700 L, 100 L to 600 L, 100 L to 500 L, 100 L to 400 L, 100 L to 300 L, 100 L to 200 L, 200 L to 1,000 L, 200 L to 900 L, 200 L to 800 L, 200 L to 700 L, 200 L to 600 L, 200 L to 500 L, 200 L to 400 L, 200 L to 300 L, 300 L to 1,000 L, 300 L to 900 L, 300 L to 800 L, 300 L to 700 L, 300 L to 600 L, 300 L to 500 L, 300 L to 400 L, 400 L to 1,000 L, 400 L to 900 L, 400 L to 800 L, 400 L to 700 L, 400 L to 600 L, 400 L to 500 L, 500 L to 1,000 L, 500 L to 900 L, 500 L to 800 L, 500 L to 700 L, 500 L to 600 L, 600 L to 1,000 L, 600 L to 900 L, 600 L to 800 L, 600 L to 700 L, 700 L to 1,000 L, 700 L to 900 L, 700 L to 800 L, 800 L to 1,000 L, 800 L to 900 L, or 900 L to 1,000 L. In some embodiments, the bioreactor has a volume of 50 L.

### 4. Cell Expansion and Stimulation

In some embodiments, the natural killer cell source, e.g., single unit of cord blood, is co-cultured with feeder cells to produce expanded and stimulated NK cells.

In some embodiments, the co-culture is carried out in a culture medium described herein, e.g., exemplary culture medium #1 (Table 1) or exemplary culture medium #2 (Table 2).

In some embodiments, the natural killer cell source, e.g., single unit of cord blood, comprises from or from about 1×10⁷ to or to about 1×10⁹ total nucleated cells prior to expansion. In some embodiments, the natural killer cell source, e.g., single unit of cord blood, comprises from or from about 1×10⁸ to or to about 1.5×10⁸ total nucleated cells prior to expansion. In some embodiments, the natural killer cell source, e.g., single unit of cord blood, comprises 1×10⁸ total nucleated cells prior to expansion. In some embodiments, the natural killer cell source, e.g., single unit of cord blood, comprises about 1×10⁸ total nucleated cells prior to expansion. In some embodiments, the natural killer cell source, e.g., single unit of cord blood, comprises 1×10⁹ total nucleated cells prior to expansion. In some embodiments, the natural killer cell source, e.g., single unit of cord blood, comprises about 1×10⁹ total nucleated cells prior to expansion.

In some embodiments, cells from the co-culture of the natural killer cell source, e.g., single unit of cord blood and feeder cells are harvested and frozen, e.g., in a cryopreservation composition described herein. In some embodiments, the frozen cells from the co-culture are an infusion-ready drug product. In some embodiments, the frozen cells from the co-culture are used as a master cell bank (MCB) from which to produce an infusion-ready drug product, e.g., through one or more additional co-culturing steps, as described herein. Thus, for example, a natural killer cell source can be expanded and stimulated as described herein to produce expanded and stimulated NK cells suitable for use in an infusion-ready drug product without generating any intermediate products. A natural killer cell source can also be expanded and stimulated as described herein to produce an intermediate product, e.g., a first master cell bank (MCB). The first MCB can be used to produce expanded and stimulated NK cells suitable for use in an infusion-ready drug product, or, alternatively, be used to produce another intermediate product, e.g., a second MCB. The second MCB can be used to produce expanded and stimulated NK cells suitable for an infusion-ready drug product, or alternatively, be used to produce another intermediate product, e.g., a third MCB, and so on.

In some embodiments, the ratio of feeder cells to cells of the natural killer cell source or MCB cells inoculated into the co-culture is from or from about 1:1 to or to about 4:1. In some embodiments, the ratio of feeder cells to cells of the natural killer cell source or MCB cells is from or from about 1:1 to or to about 3.5:1, from or from about 1:1 to or to about 3:1, from or from about 1:1 to or to about 2.5:1, from or from about 1.1 to or to about 2:1, from or from about 1:1 to or to about 1.5:1, from or from about 1.5:1 to or to about 4:1, from or from about 1.5:1 to or to about 3.5:1, from or from about 1.5:1 to or to about 3:1, from or from about 1.5:1 to or to about 2.5:1, from or from about 1.5:1 to or to about 2:1, from or from about 2:1 to or to about 4:1, from or from about 2:1 to or to about 3.5:1, from or from about 2:1 to or to about 3:1, from or from about 2:1 to or to about 2.5:1, from or from about 2.5:1 to or to about 4:1, from or from about 2.5:1 to or to about 3.5:1, from or from about 2.5:1 to or to about 3:1, from or from about 3:1 to or to about 4:1, from or from about 3:1 to or to about 3.5:1, or from or from about 3.5:1 to or to about 4:1. In some embodiments, the ratio of feeder cells to cells of the natural killer cell source or MCB inoculated into the co-culture is 2.5:1. In some embodiments, the ratio of feeder cells to cells of the natural killer cell source or MCB inoculated into the co-culture is about 2.5:1.

In some embodiments, the co-culture is carried out in a disposable culture bag, e.g., a 1L disposable culture bag. In some embodiments, the co-culture is carried out in a bioreactor, e.g., a 50 L bioreactor. In some embodiments, culture medium is added to the co-culture after the initial inoculation.

In some embodiments, the co-culture is carried out for 1, 2, 3, 4, 5, 6, 7, 8, 9, 10, 11, 12, 13, 14, 15, 16, 17, 18, 19, 20, 21, 22, 23, 24 or more days. In some embodiments, the co-culture is carried out for a maximum of 16 days.

In some embodiments, the co-culture is carried out at 37° C. or about 37° C.

In some embodiments, the co-culture is carried out at pH 7.9 or about pH 7.9.

In some embodiments, the co-culture is carried out at a dissolved oxygen (DO) level of 50% or more.

In some embodiments, exemplary culture medium #1 (Table 1) is used to produce a MCB and exemplary culture medium #2 (Table 2) is used to produce cells suitable for an infusion-ready drug product.

In some embodiments, the co-culture of the natural killer cell source, e.g., single unit of cord blood, with feeder cells yields from or from about 50×10⁸ to or to about 50×10¹² cells, e.g., MCB cells or infusion-ready drug product cells. In some embodiments, the expansion yields from or from about 50×10⁸ to or to about 25×10¹⁰, from or from about 10×10⁸ to or to about 1×10¹⁰, from or from about 50×10⁸ to or to about 75×10⁹, from or from about 50×10⁸ to or to about 50×10⁹, from or from about 50×10⁸ to or to about 25×10⁹, from or from about 50×10⁸ to or to about 1×10⁹, from or from about 50×10⁸ to or to about 75×10⁸, from or from about 75×10⁸ to or to about 50×10¹⁰, from or from about 75×10⁸ to or to about 25×10¹⁰, from or from about 75×10⁸ to or to about 1×10¹⁰, from or from about 75×10⁸ to or to about 75×10⁹, from or from about 75×10⁸ to or to about 50×10⁹, from or from about 75×10⁸ to or to about 25×10⁹, from or from about 75×10⁸ to or to about 1×10⁹, from or from about 1×10⁹ to or to about 50×10¹⁰, from or from about 1×10⁹ to or to about 25×10¹⁰, from or from about 1×10⁹ to or to about 1×10¹⁰, from or from about 1×10⁹ to or to about 75×10⁹, from or from about 1×10⁹ to or to about 50×10⁹, from or from about 1×10⁹ to or to about 25×10⁹, from or from about 25×10⁹ to or to about 50×10¹⁰, from or from about 25×10⁹ to or to about 25×10¹⁰, from or from about 25×10⁹ to or to about 1×10¹⁰, from or from about 25×10⁹ to or to about 75×10⁹, from or from about 25×10⁹ to or to about 50×10⁹, from or from about 50×10⁹ to or to about 50×10¹⁰, from or from about 50×10⁹ to or to about 25×10¹⁰, from or from about 50×10⁹ to or to about 1×10¹⁰, from or from about 50×10⁹ to or to about 75×10⁹, from or from about 75×10⁹ to or to about 50×10¹⁰, from or from about 75×10⁹ to or to about 25×10¹⁰, from or from about 75×10⁹ to or to about 1×10¹⁰, from or from about 1×10¹⁰ to or to about 50×10¹⁰, from or from about 1×10¹⁰ to or to about 25×10¹⁰, or from or from about 25×10¹⁰ to or to about 50×10¹⁰ cells, e.g., e.g., MCB cells or infusion-ready drug product cells.

In some embodiments, the expansion yields from or from about 60 to or to about 100 vials, each comprising from or from about 600 million to or to about 1 billion cells, e.g., MCB cells or infusion-ready drug product cells. In some embodiments, the expansion yields 80 or about 80 vials, each comprising or consisting of 800 million or about 800 million cells, e.g., MCB cells or infusion-ready drug product cells.

In some embodiments, the expansion yields from or from about a 100 to or to about a 500 fold increase in the number of cells, e.g., the number of MCB cells relative to the number of cells, e.g., NK cells, in the natural killer cell source. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of MCB cells relative to the number of cells, e.g., NK cells, in the natural killer cell source.

In some embodiments, the expansion yields from or from about a 100 to or to about a 70,000 fold increase in the number of cells, e.g., the number of MCB cells relative to the number of cells, e.g., NK cells, in the natural killer cell source. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of MCB cells relative to the number of cells, e.g., NK cells, in the natural killer cell source.

In some embodiments, the co-culture of the MCB cells and feeder cells yields from or from about 500 million to or to about 1.5 billion cells, e.g., NK cells suitable for use in an MCB and/or in an infusion-ready drug product. In some embodiments, the co-culture of the MCB cells and feeder cells yields from or from about 500 million to or to about 1.5 billion, from or from about 500 million to or to about 1.25 billion, from or from about 500 million to or to about 1 billion, from or from about 500 million to or to about 750 million, from or from about 750 million to or to about 1.5 billion, from or from about 500 million to or to about 1.25 billion, from or from about 750 million to or to about 1 billion, from or from about 1 billion to or to about 1.5 billion, from or from about 1 billion to or to about 1.25 billion, or from or from about 1.25 billion to or to about 1.5 billion cells, e.g., NK cells suitable for use in an MCB and/or an infusion-ready drug product.

In some embodiments, the co-culture of the MCB cells and feeder cells yields from or from about 50 to or to about 150 vials of cells, e.g., infusion-ready drug product cells, each comprising from or from about 750 million to or to about 1.25 billion cells, e.g., NK cells suitable for use in an MCB and/or an infusion-ready drug product. In some embodiments, the co-culture of the MCB cells and feeder cells yields 100 or about 100 vials, each comprising or consisting of 1 billion or about 1 billion cells, e.g., NK cells suitable for use in an MCB and/or an infusion-ready drug product.

In some embodiments, the expansion yields from or from about a 100 to or to about a 500 fold increase in the number of cells, e.g., the number of NK cells suitable for use in an MCB and/or an infusion-ready drug product relative to the number of starting MCB cells. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of NK cells suitable for use in an MCB and/or an infusion-ready drug product relative to the number of starting MCB cells.

In some embodiments, the expansion yields from or from about a 100 to or to about a 70,000 fold increase in the number of cells, e.g., the number of NK cells suitable for use in an MCB and/or an infusion-ready drug product relative to the number of starting MCB cells. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of NK cells suitable for use in an MCB and/or an infusion-ready drug product relative to the number of starting MCB cells.

In embodiments where the cells are engineered during expansion and stimulation, as described herein, not all of the expanded and stimulated cells will necessarily be engineered successfully, e.g., transduced successfully, e.g., transduced successfully with a vector comprising a heterologous protein, e.g., a heterologous protein comprising a CAR and/or IL-15 as described herein. Thus, the methods described herein can further comprise sorting engineered cells, e.g., engineered cells described herein, away from non-engineered cells.

In some embodiments, the engineered cells, e.g., transduced cells, are sorted from the non-engineered cells, e.g., the non-transduced cells using a reagent specific to an antigen of the engineered cells, e.g., an antibody that targets an antigen of the engineered cells but not the non-engineered cells. In some embodiments, the antigen of the engineered cells is a component of a CAR, e.g., a CAR described herein.

Systems for antigen-based cell separation of cells are available commercially, e.g., the CliniMACS® sorting system (Miltenyi Biotec).

In some embodiments, the engineered cells, e.g., transduced cells, are sorted from the non-engineered cells, e.g., the non-transduced cells using flow cytometry.

In some embodiments, the sorted engineered cells are used as an MCB. In some embodiments, the sorted engineered cells are used as a component in an infusion-ready drug product.

In some embodiments, the engineered cells, e.g., transduced cells, are sorted from the non-engineered cells, e.g., the non-transduced cells using a microfluidic cell sorting method. Microfluidic cell sorting methods are described, for example, in Dalili et al., “A Review of Sorting, Separation and Isolation of Cells and Microbeads for Biomedical Applications: Microfluidic Approaches,” Analyst 144:87 (2019).

In some embodiments, from or from about 1% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein. In some embodiments, from or from about 1% to or to about 90%, from or from about 1% to or to about 80%, from or from about 1% to or to about 70%, from or from about 1% to or to about 60%, from or from about 1% to or to about 50%, from or from about 1% to or to about 40%, from or from about 1% to or to about 30%, from or from about 1% to or to about 20%, from or from about 1% to or to about 10%, from or from about 1% to or to about 5%, from or from about 5% to or to about 99%, from or from about 5% to or to about 90%, from or from about 5% to or to about 80%, from or from about 5% to or to about 70%, from or from about 5% to or to about 60%, from or from about 5% to or to about 50%, from or from about 5% to or to about 40%, from or from about 5% to or to about 30%, from or from about 5% to or to about 20%, from or from about 5% to or to about 10%, from or from about 10% to or to about 99%, from or from about 10% to or to about 90%, from or from about 10% to or to about 80%, from or from about 10% to or to about 70%, from or from about 10% to or to about 60%, from or from about 10% to or to about 50%, from or from about 10% to or to about 40%, from or from about 10% to or to about 30%, from or from about 10% to or to about 20%, from or from about 20% to or to about 99%, from or from about 20% to or to about 90%, from or from about 20% to or to about 80%, from or from about 20% to or to about 70%, from or from about 20% to or to about 60%, from or from about 20% to or to about 50%, from or from about 20% to or to about 40%, from or from about 20% to or to about 30%, from or from about 30% to or to about 99%, from or from about 30% to or to about 90%, from or from about 30% to or to about 80%, from or from about 30% to or to about 70%, from or from about 30% to or to about 60%, from or from about 30% to or to about 50%, from or from about 30% to or to about 40%, from or from about 40% to or to about 99%, from or from about 40% to or to about 90%, from or from about 40% to or to about 80%, from or from about 40% to or to about 70%, from or from about 40% to or to about 70%, from or from about 40% to or to about 60%, from or from about 40% to or to about 50%, from or from about 50% to or to about 99%, from or from about 50% to or to about 90%, from or from about 50% to or to about 80%, from or from about 50% to or to about 70%, from or from about 50% to or to about 60%, from or from about 60% to or to about 99%, from or from about 60% to or to about 90%, from or from about 60% to or to about 80%, from or from about 60% to or to about 70%, from or from about 70% to or to about 99%, from or from about 70% to or to about 90%, from or from about 70% to or to about 80%, from or from about 80% to or to about 99%, from or from about 80% to or to about 90%, or from or from about 90% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein.

In some embodiments, frozen cells of a first or second MCB are thawed and cultured. In some embodiments, a single vial of frozen cells of the first or second MCB e.g., a single vial comprising 800 or about 800 million cells, e.g., first or second MCB cells, are thawed and cultured. In some embodiments, the frozen first or second MCB cells are cultured with additional feeder cells to produce cells suitable for use either as a second or third MCB or in an infusion-ready drug product. In some embodiments, the cells from the co-culture of the first or second MCB are harvested and frozen.

In some embodiments, the cells from the co-culture of the natural killer cell source, a first MCB, or a second MCB are harvested, and frozen in a cryopreservation composition, e.g., a cryopreservation composition described herein. In some embodiments, the cells are washed after harvesting. Thus, provided herein is a pharmaceutical composition comprising activated and stimulated NK cells, e.g., activated and stimulated NK cells produced by the methods described herein, e.g., harvested and washed activated and stimulated NK cells produced by the methods described herein and a cryopreservation composition, e.g., a cryopreservation composition described herein.

In some embodiments, the cells are mixed with a cryopreservation composition, e.g., as described herein, before freezing. In some embodiments, the cells are frozen in cryobags. In some embodiments, the cells are frozen in cryovials.

In some embodiments, the method further comprises isolating NK cells from the population of expanded and stimulated NK cells.

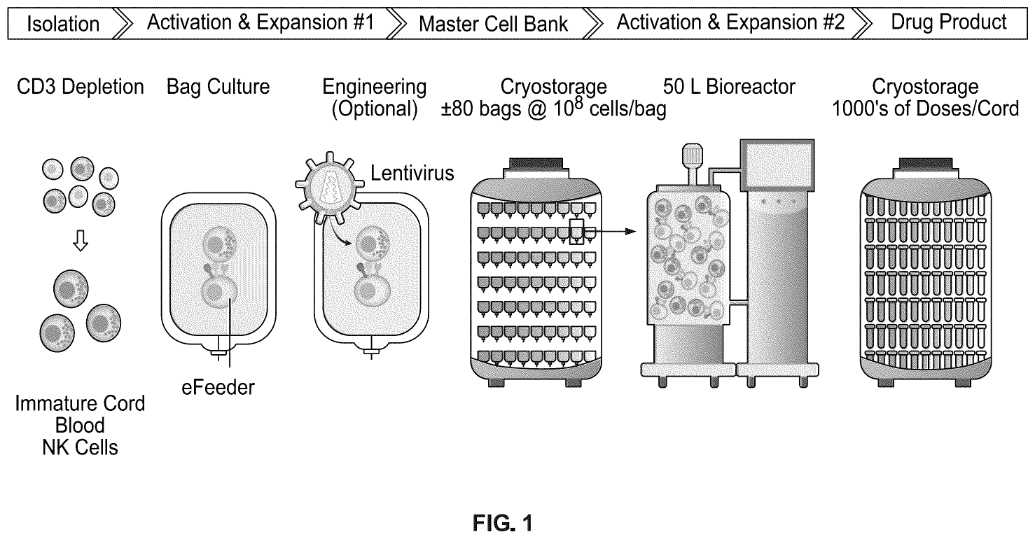

An exemplary process for expanding and stimulating NK cells is shown in FIG. 1.

### 5. Engineering

In some embodiments, the method further comprises engineering NK cell(s), e.g., to express a heterologous protein, e.g., a heterologous protein described herein, e.g., a heterologous protein comprising a CAR and/or IL-15.

In some embodiments, engineering the NK cell(s) to express a heterologous protein described herein comprises transforming, e.g., stably transforming the NK cells with a vector comprising a polynucleic acid encoding a heterologous protein described herein. Suitable vectors are described herein.

In some embodiments, engineering the NK cell(s) to express a heterologous protein described herein comprises introducing the heterologous protein via gene editing (e.g., zinc finger nuclease (ZFN) gene editing, ARCUS gene editing, CRISPR-Cas9 gene editing, or megaTAL gene editing) combined with adeno-associated virus (AAV) technology.

In some embodiments, the NK cell(s) are engineered to express a heterologous protein described herein, e.g., during or after culturing the composition in a medium comprising feeder cells.

In some embodiments, the method further comprises engineering NK cell(s), e.g., to express, over-express, knock-out, or knock-down gene(s) or gene product(s).

In some embodiments, the natural killer cells are not genetically engineered.

### E. Properties of Expanded and Stimulated NK Cells

After having been ex vivo expanded and stimulated, e.g., as described herein, the expanded and stimulated NK cell populations not only have a number/density (e.g., as described above) that could not occur naturally in the human body, but they also differ in their phenotypic characteristics, (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3− cells.

In some embodiments, the expanded and stimulated NK cells are not genetically engineered.

In some embodiments, the expanded and stimulated NK cells do not comprise a CD16 transgene.

In some embodiments, the expanded and stimulated NK cells do not express an exogenous CD16 protein.

The expanded and stimulated NK cells can be characterized, for example, by surface expression, e.g., of one or more of CD16, CD56, CD3, CD38, CD14, CD19, NKG2D, NKp46, NKp30, DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3(+) depleted, e.g., by gating on CD56+CD3− expression, but no other surface protein expression selection is carried out during expansion and stimulation.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp44+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% CD94+(KLRD1) cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD3+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD14+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CXCR+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD122+(IL2RB) cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein. Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise 50% or more, e.g., 55%, 60%, 65%, 70%, 75%, 80%, 85%, 90%, or 95% CD16+NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See, e.g., Jiao et al., “CD38: Targeted Therapy in Multiple Myeloma and Therapeutic Potential for Solid Cancers,” Expert Opinion on Investigational Drugs 29(11):1295-1308 (2020). Yet, when an anti-CD38 antibody is administered with NK cells, because NK cells naturally express CD38, they are at risk for increased fratricide. The NK cells expanded and stimulated by the methods described herein, however, express low levels of CD38 and, therefore, overcome the anticipated fratricide. While other groups have resorted to engineering methods such as genome editing to reduce CD38 expression (see, e.g., Gurney et al., “CD38 Knockout Natural Killer Cells Expressing an Affinity Optimized CD38 Chimeric Antigen Receptor Successfully Target Acute Myeloid Leukemia with Reduced Effector Cell Fratricide,” Haematologica doi:10.3324/haematol.2020.271908 (2020), the NK cells expanded and stimulated by the methods described herein express low levels of CD38 without the need for genetic engineering, which provides a surprising and unexpected benefits, e.g., for treating CD38+ cancers with the NK cells expanded and stimulated as described herein, e.g., in combination with a CD38 antibody.

Thus, in some embodiments, the expanded and stimulated NK cells, e.g., from expansion and stimulation of a single cord blood unit, e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+NK cells; and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100% NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+(KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%, e.g., less than or equal to 10%, less than or equal to 5%, less than or equal to 1% or 0% CD122+(IL2RB) cells.

In some embodiments, feeder cells do not persist in the expanded and stimulated NK cells, though, residual signature of the feeder cells may be detected, for example, by the presence of residual cells (e.g., by detecting cells with a particular surface protein expression) or residual nucleic acid and/or proteins that are expressed by the feeder cells.

For example, in some cases, the methods described herein include expanding and stimulating natural killer cells using engineered feeder cells, e.g., eHuT-78 feeder cells described above, which are engineered to express sequences that are not expressed by cells in the natural killer cell source, including the natural killer cells. For example, the engineered feeder cells can be engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

While these feeder cells may not persist in the expanded and stimulated NK cells, the expanded and stimulated NK cells may retain detectable residual amounts of cells, proteins, and/or nucleic acids from the feeder cells. Thus, their residual presence in the expanded and stimulated NK cells may be detected, for example, by detecting the cells themselves (e.g., by flow cytometry), proteins that they express, and/or nucleic acids that they express.

Thus, also described herein is a population of expanded and stimulated NK cells comprising residual feeder cells (live cells or dead cells) or residual feeder cell cellular impurities (e.g., residual feeder cell proteins or portions thereof, and/or genetic material such as a nucleic acid or portion thereof). In some cases, the expanded and stimulated NK cells comprise more than 0% and, but 0.3% or less residual feeder cells, e.g., eHuT-78 feeder cells.

In some cases, the expanded and stimulated NK cells comprise residual feeder cell nucleic acids, e.g., encoding residual 4-1BBL (UniProtKB P41273, SEQ ID NO: 1), membrane bound IL-21 (SEQ ID NO: 2), and/or mutant TNFalpha (SEQ ID NO: 3) or portion(s) thereof. In some cases, the membrane bound IL-21 comprises a CD8 transmembrane domain

In some cases, the expanded and stimulated NK cells comprise a % residual feeder cells of more than 0% and less than or equal to 0.2%, as measured, e.g., by the relative proportion of a feeder cell specific protein or nucleic acid sequence (that is, a protein or nucleic acid sequence not expressed by the natural killer cells) in the sample. For example, by qPCR, e.g., as described herein.

In some embodiments, the residual feeder cells are CD4(+) T cells. In some embodiments, the residual feeder cells are engineered CD4(+) T cells. In some embodiments, the residual feeder cell cells are engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof. Thus, in some cases, the feeder cell specific protein is 4-1BBL (UniProtKB P41273, SEQ ID NO: 1), membrane bound IL-21 (SEQ ID NO: 2), and/or mutant TNFalpha (SEQ ID NO: 3). And, therefore, the feeder cell specific nucleic acid is a nucleic acid encoding 4-1BBL (UniProtKB P41273, SEQ ID NO: 1), membrane bound IL-21 (SEQ ID NO: 2), and/or mutant TNFalpha (SEQ ID NO: 3), or portion thereof. In some cases, the membrane bound IL-21 comprises a CD8 transmembrane domain.

A wide variety of different methods can be used to analyze and detect the presence of nucleic acids or protein gene products in a biological sample. As used herein, “detecting” can refer to a method used to discover, determine, or confirm the existence or presence of a compound and/or substance (e.g., a cell, a protein and/or a nucleic acid). In some embodiments, a detecting method can be used to detect a protein. In some embodiments, detecting can include chemiluminescence or fluorescence techniques. In some embodiments, detecting can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, detecting can include immunoprecipitation of the protein (Jungblut et al., J Biotechnol. 31; 41(2-3):111-20 (1995); Franco et al., Eur J Morphol. 39(1):3-25 (2001)). In some embodiments, a detecting method can be used to detect a nucleic acid (e.g., DNA and/or RNA). In some embodiments, detecting can include Northern blot analysis, nuclease protection assays (NPA), in situ hybridization, or reverse transcription-polymerase chain reaction (RT-PCR) (Raj et al., Nat. Methods 5, 877-879 (2008); Jin et al., J Clin Lab Anal. 11(1):2-9 (1997); Ahmed, J Environ Sci Health C Environ Carcinog Ecotoxicol Rev. 20(2):77-116 (2002)).

Thus, also described herein, are methods for detecting a population of expanded and stimulated NK cells, e.g., expanded and stimulated using the methods described herein, that have been co-cultured with engineered feeder cells, e.g., eHuT-78 feeder cells described herein.

### II. Natural Killer Cell Engineering

In some embodiments, the natural killer cells are engineered, e.g., to produce CAR-NK(s) and/or IL-15 expressing NK(s).

In some embodiments, the natural killer cells are engineered, e.g., transduced, during expansion and stimulation, e.g., expansion and stimulation described herein. In some embodiments, the natural killer cells are engineered during expansion and stimulation, e.g., during production of a MCB, as described herein. In some embodiments, the natural killer cells are engineered during expansion and stimulation, e.g., during production of NK cells suitable for use in an injection-ready drug product and/or during production of a MCB, as described above. Thus, in some embodiments, the NK cell(s) are host cells and provided herein are NK host cell(s) expressing a heterogeneous protein, e.g., as described herein.

In some embodiments, the natural killer cells are engineered prior to expansion and stimulation. In some embodiments, the natural killer cells are engineered after expansion and stimulation.

In some embodiments, the NK cells are engineered by transducing with a vector. Suitable vectors are described herein, e.g., lentiviral vectors, e.g., a lentiviral vectors comprising a heterologous protein, e.g., as described herein. In some embodiments, the NK cells are transduced during production of a first MCB, as described herein.

In some embodiments, the NK cell(s) are transduced at a multiplicity of infection of from or from about 1 to or to about 40 viral particles per cell. In some embodiments, the NK cell(s) are transduced at a multiplicity of infection of or of about 1, of or of about 5, of or of about 10, of or of about 15, of or of about 20, of or of about 25, of or of about 30, of or of about 35, or of or of about 40 viral particles per cell.

### A. Chimeric Antigen Receptors

In some embodiments, the heterologous protein is a fusion protein, e.g., a fusion protein comprising a chimeric antigen receptor (CAR) is introduced into the NK cell, e.g., during the expansion and stimulation process.

In some embodiments, the CAR comprises one or more of: a signal sequence, an extracellular domain, a hinge, a transmembrane domain, and one or more intracellular signaling domain sequences. In some embodiments, the CAR further comprises a spacer sequence.

In some embodiments, the CAR comprises (from N- to C-terminal): a signal sequence, an extracellular domain, a hinge, a spacer, a transmembrane domain, a first signaling domain sequence, a second signaling domain sequence, and a third signaling domain sequence.

In some embodiments, the CAR comprises (from N- to C-terminal): a signal sequence, an extracellular domain, a hinge, a transmembrane domain, a first signaling domain sequence, a second signaling domain sequence, and a third signaling domain sequence.

In some embodiments the extracellular domain comprises an antibody or antigen-binding portion thereof.

In some embodiments, one or more of the intracellular signaling domain sequence(s) is a CD28 intracellular signaling sequence. In some embodiments, the CD28 intracellular signaling sequence comprises or consists of SEQ ID NO: 5.

In some embodiments, one or more of the intracellular signaling domain sequence(s) is an OX40L signaling sequence. In some embodiments, the OX40L signaling sequence comprises or consists of SEQ ID NO: 8.

In some embodiments, one or more of the intracellular signaling sequence(s) is a CD3ζ intracellular signaling domain sequence. In some embodiments, the CD3ζ intracellular signaling sequence comprises of consists of SEQ ID NO: 11.

In some embodiments, the CAR comprises a CD28 intracellular signaling sequence (SEQ ID NO: 5), an OX40L intracellular signaling sequence (SEQ ID NO: 8), and a CD3ζ intracellular signaling sequence (SEQ ID NO: 11).

In some embodiments, the CAR comprises an intracellular signaling domain comprising or consisting of SEQ ID NO: 19.

In some embodiments, the CAR does not comprise an OX40L intracellular signaling domain sequence.

In some embodiments, the CAR comprises a CD28 intracellular signaling sequence (SEQ ID NO: 5), and a CD3ζ intracellular signaling sequence (SEQ ID NO: 11), but not an OX40L intracellular signaling domain sequence.

### B. IL-15

In some embodiments, the NK cell is engineered to express IL-15, e.g., human IL-15 (UniProtKB #P40933; NCBI Gene ID #3600), e.g., soluble human IL-15 or an ortholog thereof, or a variant of any of the foregoing. In some embodiments, the IL-15 is expressed as part of a fusion protein further comprising a cleavage site. In some embodiments, the IL-15 is expressed as part of a polyprotein comprising a T2A ribosomal skip sequence site (sometimes referred to as a self-cleaving site).

In some embodiments, the IL-15 comprises or consists of SEQ ID NO: 16.

In some embodiments, the T2A cleavage site comprises or consists of SEQ ID NO: 14.

In some embodiments, the IL-15 is expressed as part of a fusion protein comprising a CAR, e.g., a CAR described herein.

In some embodiments, the fusion protein comprises (oriented from N-terminally to C-terminally): a CAR comprising, a cleavage site, and IL-15.

In some embodiments, the fusion protein comprises SEQ ID NO: 20.

### C. Inhibitory Receptors

In some embodiments, the NK cell is engineered to alter, e.g., reduce, expression of one or more inhibitor receptor genes.

In some embodiments, the inhibitory receptor gene is a HLA-specific inhibitory receptor. In some embodiments, the inhibitory receptor gene is a non-HLA-specific inhibitory receptor.

In some embodiments, the inhibitor receptor gene is selected from the group consisting of KIR, CD94/NKG2A, LILRB1, PD-1, IRp60, Siglec-7, LAIR-1, and combinations thereof.

### D. Polynucleic Acids, Vectors, and Host Cells

Also provided herein are polynucleic acids encoding the fusion protein(s) or portions thereof, e.g., the polynucleotide sequences encoding the polypeptides described herein, as shown in the Table of sequences provided herein

Also provided herein are vector(s) comprising the polynucleic acids, and cells, e.g., NK cells, comprising the vector(s).

In some embodiments, the vector is a lentivirus vector. See, e.g., Milone et al., “Clinical Use of Lentiviral Vectors,” Leukemia 32:1529-41 (2018). In some embodiments, the vector is a retrovirus vector. In some embodiments, the vector is a gamma retroviral vector. In some embodiments, the vector is a non-viral vector, e.g., a piggyback non-viral vector (PB transposon, see, e.g., Wu et al., “piggyback is a Flexible and Highly Active Transposon as Compared to Sleeping Beauty, Tol2, and Mosi in Mammalian Cells,” PNAS 103(41):15008-13 (2006)), a sleeping beauty non-viral vector (SB transposon, see, e.g., Hudecek et al., “Going Non-Viral: the Sleeping Beauty Transposon System Breaks on Through to the Clinical Side,” Critical Reviews in Biochemistry and Molecular Biology 52(4):355-380 (2017)), or an mRNA vector.

### III. Cryopreservation

### A. Cryopreservation Compositions

Provided herein are cryopreservation compositions, e.g., cryopreservation compositions suitable for intravenous administration, e.g., intravenous administration of NK cells, e.g., the NK cells described herein. In some embodiments, a pharmaceutical composition comprises the cryopreservation composition and cells, e.g., the NK cells described herein.

### 1. Albumin

In some embodiments, the cryopreservation composition comprises albumin protein, e.g., human albumin protein (UniProtKB Accession P0278, SEQ ID NO: 21) or variant thereof. In some embodiments, the cryopreservation composition comprises an ortholog of an albumin protein, e.g., human albumin protein, or variant thereof. In some embodiments, the cryopreservation composition comprises a biologically active portion of an albumin protein, e.g., human albumin, or variant thereof.

In some embodiments, the albumin, e.g., human albumin, is provided as a solution, also referred to herein as an albumin solution or a human albumin solution. Thus, in some embodiments, the cryopreservation composition is or comprises an albumin solution, e.g., a human albumin solution. In some embodiments, the albumin solution is a serum-free albumin solution.

In some embodiments, the albumin solution is suitable for intravenous use.

In some embodiments, the albumin solution comprises from or from about 40 to or to about 200 g/L albumin. In some embodiments, the albumin solution comprises from or from about 40 to or to about 50 g/L albumin, e.g., human albumin. In some embodiments, the albumin solution comprises about 200 g/L albumin, e.g., human albumin. In some embodiments, the albumin solution comprises 200 g/L albumin, e.g., human albumin.

In some embodiments, the albumin solution comprises a protein composition, of which 95% or more is albumin protein, e.g., human albumin protein. In some embodiments, 96%, 97%, 98%, or 99% or more of the protein is albumin, e.g., human albumin.

In some embodiments, the albumin solution further comprises sodium. In some embodiments, the albumin solution comprises from or from about 100 to or to about 200 mmol sodium. In some embodiments, the albumin solution comprises from or from about 130 to or to about 160 mmol sodium.

In some embodiments, the albumin solution further comprises potassium. In some embodiments, the albumin solution comprises 3 mmol or less potassium. In some embodiments, the albumin solution further comprises 2 mmol or less potassium.

In some embodiments, the albumin solution further comprises one or more stabilizers. In some embodiments, the stabilizer(s) are selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan). In some embodiments, the solution comprises less than 0.1 mmol of each of the one or more stabilizers per gram of protein in the solution. In some embodiments, the solution comprises from or from about 0.05 to or to about 0.1, e.g., from or from about 0.064 to or to about 0.096 mmol of each of the stabilizers per gram of protein in the solution. In some embodiments, the solution comprises less than 0.1 mmol of total stabilizer per gram of protein in the solution. In some embodiments, the solution comprises from or from about 0.05 to or to about 0.1, e.g., from or from about 0.064 to or to about 0.096 mmol of total stabilizer per gram of protein in the solution.

In some embodiments, the albumin solution consists of a protein composition, of which 95% or more is albumin protein, sodium, potassium, and one or more stabilizers selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan) in water.

In some embodiments, the cryopreservation composition comprises from or from about 10% v/v to or to about 50% v/v of an albumin solution, e.g., an albumin solution described herein. In some embodiments, the cryopreservation composition comprises from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of an albumin solution described herein. In some embodiments, the cryopreservation composition comprises about 10%, about 15%, about 20%, about 25%, about 30%, about 35%, about 40%, about 45%, or about 50% v/v of an albumin solution described herein. In some embodiments, the cryopreservation composition comprises 10%, 15%, 20%, 25%, 30%, 35%, 40%, 45%, or 50% v/v of an albumin solution described herein.

In some embodiments, the cryopreservation composition comprises from or from about 20 to or to about 100 g/L albumin, e.g., human albumin. In some embodiments, the cryopreservation composition comprises from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 100, from or from about 80 to or to about 90, or from or from about 90 to or to about 100 g/L albumin, e.g., human albumin.

In some embodiments, the cryopreservation composition comprises 20 g/L albumin, e.g., human albumin. In some embodiments, the cryopreservation composition comprises 40 g/L albumin, e.g., human albumin. In some embodiments, the cryopreservation composition comprises 70 g/L albumin, e.g., human albumin. In some embodiments, the cryopreservation composition comprises 100 g/L albumin, e.g., human albumin.

In some embodiments, the cryopreservation composition comprises about 20 g/L albumin, e.g., human albumin. In some embodiments, the cryopreservation composition comprises about 40 g/L albumin, e.g., human albumin. In some embodiments, the cryopreservation composition comprises about 70 g/L albumin, e.g., human albumin. In some embodiments, the cryopreservation composition comprises about 100 g/L albumin, e.g., human albumin.

In some embodiments, the cryopreservation composition further comprises a stabilizer, e.g., an albumin stabilizer. In some embodiments, the stabilizer(s) are selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan). In some embodiments, the cryopreservation composition comprises less than 0.1 mmol of each of the one or more stabilizers per gram of protein, e.g., per gram of albumin protein, in the composition. In some embodiments, the cryopreservation composition comprises from or from about 0.05 to or to about 0.1, e.g., from or from about 0.064 to or to about 0.096 mmol of each of the stabilizers per gram of protein, e.g., per gram of albumin protein in the composition. In some embodiments, the cryopreservation composition comprises less than 0.1 mmol of total stabilizer per gram of protein, e.g., per gram of albumin protein in the cryopreservation composition. In some embodiments, the cryopreservation composition comprises from or from about 0.05 to or to about 0.1, e.g., from or from about 0.064 to or to about 0.096 mmol of total stabilizer per gram of protein, e.g., per gram of albumin protein, in the cryopreservation composition.

### 2. Dextran

In some embodiments, the cryopreservation composition comprises Dextran, or a derivative thereof.

Dextran is a polymer of anhydroglucose composed of approximately 95% α-D-(1-6) linkages (designated (C₆H₁₀O₅)ₙ). Dextran fractions are supplied in molecular weights of from about 1,000 Daltons to about 2,000,000 Daltons. They are designated by number (Dextran X), e.g., Dextran 1, Dextran 10, Dextran 40, Dextran 70, and so on, where X corresponds to the mean molecular weight divided by 1,000 Daltons. So, for example, Dextran 40 has an average molecular weight of or about 40,000 Daltons.

In some embodiments, the average molecular weight of the dextran is from or from about 1,000 Daltons to or to about 2,000,000 Daltons. In some embodiments, the average molecular weight of the dextran is or is about 40,000 Daltons. In some embodiments, the average molecular weight of the dextran is or is about 70,000 Daltons.

In some embodiments, the dextran is selected from the group consisting of Dextran 40, Dextran 70, and combinations thereof. In some embodiments, the dextran is Dextran 40.

In some embodiments, the dextran, e.g., Dextran 40, is provided as a solution, also referred to herein as a dextran solution or a Dextran 40 solution. Thus, in some embodiments, the composition comprises a dextran solution, e.g., a Dextran 40 solution.

In some embodiments, the dextran solution is suitable for intravenous use.

In some embodiments, the dextran solution comprises about 5% to about 50% w/w dextran, e.g., Dextran 40. In some embodiments, the dextran solution comprises from or from about 5% to or to about 50%, from or from about 5% to or to about 45%, from or from about 5% to or to about 40%, from or from about 5% to or to about 35%, from or from about 5% to or to about 30%, from or from about 5% to or to about 25%, from or from about 5% to or to about 20%, from or from about 5% to or to about 15%, from or from about 5% to or to about 10%, from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% w/w dextran, e.g., Dextran 40. In some embodiments, the dextran solution comprises 5%, 10%, 15%, 20%, 25%, 30%, 35%, 40%, 45%, or 50% w/w dextran, e.g., Dextran 40. In some embodiments, the dextran solution comprises about 5%, about 10%, about 15%, about 20%, about 25%, about 30%, about 35%, about 40%, about 45%, or about 50% w/w dextran, e.g., Dextran 40.

In some embodiments, the dextran solution comprises from or from about 25 g/L to or to about 200 g/L dextran, e.g., Dextran 40. In some embodiments, the dextran solution comprises from or from about 35 to or to about 200, from or from about 25 to or to about 175, from or from about 25 to or to about 150, from or from about 25 to or to about 125, from or from about 25 to or to about 100, from or from about 25 to or to about 75, from or from about 25 to or to about 50, from or from about 50 to or to about 200, from or from about 50 to or to about 175, from or from about 50 to or to about 150, from or from about 50 to or to about 125, from or from about 50 to or to about 100, from or from about 50 to or to about 75, from or from about 75 to or to about 200, from or from about 75 to or to about 175, from or from about 75 to or to about 150, from or from about 75 to or to about 125, from or from about 75 to or to about 100, from or from about 100 to or to about 200, from or from about 100 to or to about 175, from or from about 100 to or to about 150, from or from about 100 to or to about 125, from or from about 125 to or to about 200, from or from about 125 to or to about 175, from or from about 125 to or to about 150, from or from about 150 to or to about 200, from or from about 150 to or to about 175, or from or from about 175 to or to about 200 g/L dextran e.g., Dextran 40. In some embodiments, the dextran solution comprises 25, 50, 75, 100, 125, 150, 175, or 200 g/L dextran, e.g., Dextran 40. In some embodiments, the dextran solution comprises 100 g/L dextran, e.g., Dextran 40. In some embodiments, the dextran solution comprises about 25, about 50, about 75, about 100, about 125, about 150, about 175, or about 200 g/L dextran, e.g., Dextran 40. In some embodiments, the dextran solution comprises about 100 g/L dextran, e.g., Dextran 40.

In some embodiments, the dextran solution further comprises glucose (also referred to as dextrose). In some embodiments, the dextran solution comprises from or from about 10 g/L to or to about 100 g/L glucose. In some embodiments, the dextran solution comprises from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 90, from or from about 80 to or to about 100, from or from about 80 to or to about 90, or from or from about 90 to or to about 100 g/L glucose. In some embodiments, the dextran solution comprises 10, 20, 30, 40, 50, 60, 70, 80, 90, or 100 g/L glucose. In some embodiments, the dextran solution comprises 50 g/L glucose. In some embodiments, the dextran solution comprises about 10, about 20, about 30, about 40, about 50, about 60, about 70, about 80, about 90, or about 100 g/L glucose. In some embodiments, the dextran solution comprises 50 g/L glucose.

In some embodiments, the dextran solution consists of dextran, e.g., Dextran 40, and glucose in water.

In some embodiments, the cryopreservation composition comprises from or from about 10% v/v to or to about 50% v/v of a dextran solution described herein. In some embodiments, the cryopreservation composition comprises from or from about 10% to 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of a dextran solution, e.g., a dextran solution described herein. In some embodiments, the cryopreservation composition comprises 10%, 15%, 20%, 25%, 30%, 35%, 40%, 45%, or 50% v/v of a dextran solution, e.g., a dextran solution described herein. In some embodiments, the cryopreservation composition comprises about 10%, about 15%, about 20%, about 25%, about 30%, about 35%, about 40%, about 45%, or about 50% v/v of a dextran solution, e.g., a dextran solution described herein.

In some embodiments, the cryopreservation composition comprises from or from about 10 to or to about 50 g/L dextran, e.g., Dextran 40. In some embodiments, the cryopreservation composition comprises from or from about 10 to or to about 50, from or from about 10 to or to about 45, from or from about 10 to or to about 40, from or from about 10 to or to about 35, from or from about 10 to or to about 30, from or from about 10 to or to about 25, from or from about 10 to or to about 20, from or from about 10 to or to about 15, from or from about 15 to or to about 50, from or from about 15 to or to about 45, from or from about 15 to or to about 40, from or from about 15 to or to about 35, from or from about 15 to or to about 30, from or from about 15 to or to about 25, from or from about 15 to or to about 20, from or from about 20 to or to about 50, from or from about 20 to or to about 45, from or from about 20 to or to about 40, from or from about 20 to or to about 30, from or from about 20 to or to about 25, from or from about 25 to or to about 50, from or from about 25 to or to about 45, from or from about 25 to or to about 40, from or from about 25 to or to about 35, from or from about 25 to or to about 30, from or from about 30 to or to about 50, from or from about 30 to or to about 45, from or from about 30 to or to about 40, from or from about 30 to or to about 35, from or from about 35 to or to about 50, from or from about 35 to or to about 45, from or from about 35 to or to about 40, from or from about 40 to or to about 50, from or from about 40 to or to about 45, or from or from about 45 to or to about 50 g/L dextran, e.g., Dextran 40. In some embodiments, the cryopreservation composition comprises 10, 15, 20, 25, 30, 30, 35, 40, 45, or 50 g/L dextran, e.g., Dextran 40. In some embodiments, the cryopreservation composition comprises about 10, about 15, about 20, about 25, about 30, about 30, about 35, about 40, about 45, or about 50 g/L dextran, e.g., Dextran 40.

### 3. Glucose

In some embodiments, the cryopreservation composition comprises glucose.

In some embodiments, as described above, the cryopreservation composition comprises a Dextran solution comprising glucose.

In some embodiments, the cryopreservation composition comprises a Dextran solution that does not comprise glucose. In some embodiments, e.g., when the Dextran solution does not comprise glucose, glucose is added separately to the cryopreservation composition.

In some embodiments, the cryopreservation composition comprises from or from about 5 to or to about 25 g/L glucose. In some embodiments, the cryopreservation composition comprises from or from about 5 to or to about 25, from or from about 5 to or to about 20, from or from about 5 to or to about 15, from or from about 5 to or to about 10, from or from about 10 to or to about 25, from or from about 10 to or to about 20, from or from about 10 to or to about 15, from or from about 15 to or to about 25, from or from about 15 to or to about 20, or from or from about 20 to or to about 25 g/L glucose. In some embodiments, the cryopreservation composition comprises 5, 7.5, 10, 12.5, 15, 17.5, 20, 22.5, or 25 g/L glucose. In some embodiments, the cryopreservation composition comprises 12.5 g/L glucose. In some embodiments, the cryopreservation composition comprises about 5, about 7.5, about 10, about 12.5, about 15, about 17.5, about 20, about 22.5, or about 25 g/L glucose. In some embodiments, the cryopreservation composition comprises about 12.5 g/L glucose.

In some embodiments, the cryopreservation composition comprises less than 2.75% w/v glucose. In some embodiments, the cryopreservation composition comprises less than 27.5 g/L glucose. In some embodiments, the cryopreservation composition comprises less than 2% w/v glucose. In some embodiments, the cryopreservation composition comprises less than 1.5% w/v glucose. In some embodiments, the cryopreservation composition comprises about 1.25% w/v or less glucose.

### 4. Dimethyl Sulfoxide

In some embodiments, the cryopreservation composition comprises dimethyl sulfoxide (DMSO, also referred to as methyl sulfoxide and methylsulfinylmethane).

In some embodiments, the DMSO is provided as a solution, also referred to herein as a DMSO solution. Thus, in some embodiments, the cryopreservation composition comprises a DMSO solution.

In some embodiments, the DMSO solution is suitable for intravenous use.

In some embodiments, the DMSO solution comprises 1.1 g/mL DMSO. In some embodiments, the DMSO solution comprises about 1.1 g/mL DMSO.

In some embodiments, the cryopreservation composition comprises from or from about 1% to or to about 10% v/v of the DMSO solution. In some embodiments, the cryopreservation composition comprises from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 5% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v of the DMSO solution. In some embodiments, the cryopreservation composition comprises 1%, 2%, 3%, 4%, 5%, 6%, 7%, 8%, 9%, or 10% v/v of the DMSO solution. In some embodiments, the cryopreservation composition comprises 5% of the DMSO solution. In some embodiments, the cryopreservation composition comprises about 1%, about 2%, about 3%, about 4%, about 5%, about 6%, about 7%, about 8%, about 9%, or about 10% v/v of the DMSO solution. In some embodiments, the cryopreservation composition comprises about 5% of the DMSO solution.

In some embodiments, the cryopreservation composition comprises from or from about 11 to or to about 110 g/L DMSO. In some embodiments, from or from about the cryopreservation composition comprises from or from about 11 to or to about 110, from or from about 11 to or to about 99, from or from about 11 to or to about 88, from or from about 11 to or to about 77, from or from about 11 to or to about 66, from or from about 11 to or to about 55, from or from about 11 to or to about 44, from or from about 11 to or to about 33, from or from about 11 to or to about 22, from or from about 22 to or to about 110, from or from about 22 to or to about 99, from or from about 22 to or to about 88, from or from about 22 to or to about 77, from or from about 22 to or to about 77, from or from about 22 to or to about 66, from or from about 22 to or to about 55, from or from about 22 to or to about 44, from or from about 22 to or to about 33, from or from about 33 to or to about 110, from or from about 33 to or to about 99, from or from about 33 to or to about 88, from or from about 33 to or to about 77, from or from about 33 to or to about 66, from or from about 33 to or to about 55, from or from about 33 to or to about 44, from or from about 44 to or to about 110, from or from about 44 to or to about 99, from or from about 44 to or to about 88, from or from about 44 to or to about 77, from or from about 44 to or to about 66, from or from about 44 to or to about 55, from or from about 55 to or to about 110, from or from about 55 to or to about 99, from or from about 55 to or to about 88, from or from about 55 to or to about 77, from or from about 55 to or to about 66, from or from about 66 to or to about 110, from or from about 66 to or to about 99, from or from about 66 to or to about 88, from or from about 66 to or to about 77, from or from about 77 to or to about 119, from or from about 77 to or to about 88, from or from about 88 to or to about 110, from or from about 88 to or to about 99, or from or from about 99 to or to about 110 g/L DMSO. In some embodiments, the cryopreservation composition comprises 11, 22, 33, 44, 55, 66, 77, 88, 99, or 110 g/L DMSO. In some embodiments, the cryopreservation composition comprises 55 g/L DMSO. In some embodiments, the cryopreservation composition comprises about 11, about 22, about 33, about 44, about 55, about 66, about 77, about 88, about 99, or about 110 g/L DMSO. In some embodiments, the cryopreservation composition comprises about 55 g/L DMSO.

### 5. Buffers

In some embodiments, the cryopreservation composition comprises a buffer solution, e.g., a buffer solution suitable for intravenous administration.

Buffer solutions include, but are not limited to, phosphate buffered saline (PBS), Ringer's Solution, Tyrode's buffer, Hank's balanced salt solution, Earle's Balanced Salt Solution, saline, and Tris.

In some embodiments, the buffer solution is phosphate buffered saline (PBS).

### 6. Exemplary Cryopreservation Compositions

In some embodiments, the cryopreservation composition comprises or consists of: 1) albumin, e.g., human albumin, 2) dextran, e.g., Dextran 40, 3) DMSO, and 4) a buffer solution. In some embodiments, the cryopreservation composition further comprises glucose. In some embodiments, the cryopreservation composition consists of 1) albumin, e.g., human albumin, 2) dextran, e.g., Dextran 40, 3) glucose, 4) DMSO, and 5) a buffer solution.

In some embodiments, the cryopreservation composition comprises: 1) an albumin solution described herein, 2) a dextran solution described herein, 3) a DMSO solution described herein, and 4) a buffer solution.

In some embodiments, the cryopreservation composition consists of: 1) an albumin solution described herein, 2) a dextran solution described herein, 3) a DMSO solution described herein, and 4) a buffer solution.

In some embodiments, the cryopreservation composition does not comprise a cell culture medium.

In one embodiment, the cryopreservation composition comprises or comprises about 40 mg/mL human albumin, 25 mg/mL Dextran 40, 12.5 mg/mL glucose, and 55 mg/mL DMSO.

In one embodiment, the cryopreservation composition comprises or comprises about or consists of or consists of about 40 mg/mL human albumin, 25 mg/mL Dextran 40, 12.5 mg/mL glucose, 55 mg/mL DMSO, and 0.5 mL/mL 100% phosphate buffered saline (PBS) in water.

In one embodiment, the cryopreservation composition comprises or comprises about 32 mg/mL human albumin, 25 mg/mL Dextran 40, 12.5 mg/mL glucose, and 55 mg/mL DMSO.

In one embodiment, the cryopreservation composition comprises or comprises about or consists of or consists of about 32 mg/mL human albumin, 25 mg/mL Dextran 40, 12.5 mg/mL glucose, 55 mg/mL DMSO, and 0.54 mL/mL 100% phosphate buffered saline (PBS) in water.

Exemplary Cryopreservation Compositions are shown in Table 3.

TABLE 3
Exemplary Cryopreservation Compositions

| | | | Exemplary Range |
|---|---|---|---|
| | Concentration | Exemplary | v/v % in |
| Excipient | Range of | Solution | Cryopreservation |
| Solution | Solution | Concentration | Composition |
| Albumin | 40-200 g/L albumin | 200 g/L albumin | 10%-50% |
| Solution | in water | | |
| Dextran 40 | 25-200 g/L Dextran | 100 g/L Dextran | 10%-50% |
| Solution | 40; and 0-100 g/L | 40; 50 g/L | |
| | glucose in water | glucose | |
| DMSO | 11-110 g/L DMSO | 1,100 g/L DMSO | 1%-10% |
| | in water | | |
| Buffer | to volume | to volume | to volume |

TABLE 4
Exemplary Cryopreservation Compositions #1

| | | Exemplary | Final |
|---|---|---|---|
| | | v/v % in Cryo- | Concentration in |
| Excipient | Solution | preservation | Cryopreservation |
| Solution | Composition | Composition #1 | Composition #1 |
| Albumin | 200 g/L albumin | 20% | 40 mg/mL albumin |
| Solution | in water | | |
| Dextran 40 | 100 g/L Dextran | 25% | 25 mg/mL Dextran |
| Solution | 40; and 50 g/L | | 40; 12.5 mg/mL |
| | glucose in water | | glucose |
| DMSO | 100% DMSO | 5% | 55 mg/mL |
| | (1,100 g/L) | | |
| Buffer | 100% Phosphate | 50% | 0.5 mL/mL |
| | Buffered Saline | | |
| | (PBS) | | |

TABLE 5
Exemplary Cryopreservation Cryopreservation #2

| | | Exemplary | Final |
|---|---|---|---|
| | | v/v % in Cryo- | Concentration in |
| Excipient | Solution | preservation | Cryopreservation |
| Solution | Composition | Composition #2 | Composition #2 |
| Albumin | 200 g/L albumin | 16% | 32 mg/mL albumin |
| Solution | in water | | |
| Dextran 40 | 100 g/L Dextran | 25% | 25 mg/mL Dextran |
| Solution | 40; and 50 g/L | | 40; 12.5 mg/mL |
| | glucose in water | | glucose |
| DMSO | 100% DMSO | 5% | 55 mg/mL |
| | (1,100 g/L) | | |
| Buffer | 100% Phosphate | 54% | 0.54 mL/mL |
| | Buffered Saline | | |
| | (PBS) | | |

### B. Methods of Cryopreserving

The cryopreservation compositions described herein can be used for cryopreserving cell(s), e.g., therapeutic cells, e.g., natural killer (NK) cell(s), e.g., the NK cell(s) described herein.

In some embodiments, the cell(s) are an animal cell(s). In some embodiments, the cell(s) are human cell(s).

In some embodiments, the cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein.

In some embodiments, cryopreserving the cell(s) comprises: mixing the cell(s) with a cryopreservation composition or components thereof described herein to produce a composition, e.g., a pharmaceutical composition; and freezing the mixture.

In some embodiments, cryopreserving the cell(s) comprises: mixing a composition comprising the cell(s) with a cryopreservation composition or components thereof described herein to produce a composition, e.g., a pharmaceutical composition; and freezing the mixture. In some embodiments, the composition comprising the cell(s) comprises: the cell(s) and a buffer. Suitable buffers are described herein.

In some embodiments, cryopreserving the cell(s) comprises: mixing a composition comprising the cell(s) and a buffer, e.g., PBS, with a composition comprising albumin, Dextran, and DMSO, e.g., as described herein; and freezing the mixture.

In some embodiments, cryopreserving the cell(s) comprises: mixing a composition comprising the cell(s) and a buffer, e.g., PBS 1:1 with a composition comprising 40 mg/mL albumin, e.g., human albumin, 25 mg/mL Dextran, e.g., Dextran 40, 12.5 mg/mL glucose and 55 mg/mL DMSO.

In some embodiments, the composition comprising the cell(s) and the buffer, e.g., PBS, comprises from or from about 2×10⁷ to or to about 2×10⁹ cells/mL. In some embodiments, the composition comprising the cell(s) and the buffer, e.g., PBS, comprises 2×10⁸ cells/mL. In some embodiments, the composition comprising the cell(s) and the buffer, e.g., PBS, comprising about 2×10⁸ cells/mL.

In some embodiments, cryopreserving the cell(s) comprising mixing: the cell(s), a buffer, e.g., PBS, albumin, e.g., human albumin, Dextran, e.g., Dextran 40, and DMSO; and freezing the mixture.

In some embodiments, the mixture comprises from or from about 1×10⁷ to or to about 1×10⁹ cells/mL. In some embodiments, the mixture comprises 1×10⁸ cells/mL. In some embodiments, the mixture comprises about 1×10⁸ cells/mL.

Suitable ranges for albumin, Dextran, and DMSO are set forth above.

In some embodiments, the composition is frozen at or below −135° C.

In some embodiments, the composition is frozen at a controlled rate.

### IV. Antibodies

In some embodiments, the HER2 targeting antibody is an EGFR targeting antibody selected from Table 6, or a combination thereof.

TABLE 6
Exemplary HER2 Targeting Antibodies

| Name | Internal Name | Antigen | Company | Reference |
|---|---|---|---|---|
| trastuzumab | Herceptin, RG597, | HER2/neu | Genentech, Roche | Nemeth et al., Br J |
| | anti-p185-HER2, | | | Pharmacol. 2017 November; |
| | huMAb4D5-8, | | | 174(21): 3727-3748 |
| | rhuMAb HER2 | | | |
| margetuximab | MARGENZA, | HER2/neu | MacroGenics, Zai | Catenacci et al., Lancet |
| | MGAH22, 4D5 | | Lab | Oncol. 2020 August; |
| | | | | 21(8): 1066-1076 |
| pertuzumab | Perjeta, rhuMAb 2C4, | HER2/neu | Genentech | Gianni et al., Lancet |
| | RG1273 | | | Oncol. 2012 January; |
| | | | | 13(1): 25-32 |
| trastuzumab | Kadcyla, PRO132365, | HER2/neu | Genentech, | Minckwitz et al., N Engl |
| emtansine | trastuzumab-MCC- | | ImmunoGen, | J Med. 2019 Feb. 14; |
| | DM1, Trastuzumab- | | PDL, Roche | 380(7): 617-628 |
| | DM1, T-DM1, | | | |
| | RG3502 | | | |
| PF-05280014 | Trazimera, | HER2/neu | Pfizer | Pegram et al., Br J |
| | Trastuzumab-Pfizer | | | Cancer. 2019 January; |
| | | | | 120(2): 172-182 |
| trastuzumab- | Kanjinti, ABP 980 | HER2/neu | Allergan, Amgen | Trial ID: NCT04644406 |
| anns | | | | |
| HLX02 | Zercepac, Han Quyou | HER2/neu | Shanghai Henlius | Zhu et al. Cancer |
| | | | | Chemother Pharmacol. |
| | | | | 2021 March; 87(3): |
| | | | | 349-359, |
| trastuzumab- | Ogivri, Hercules, | HER2/neu | Biocon, Mylan | Waller et al., Br J Clin |
| dkst | Myl1401O, Myl- | | | Pharmacol. 2018 October; |
| | 1401O, Bmab-200, | | | 84(10): 2336-2343 |
| | CANMAb | | | |
| Hervycta | | HER2/neu | Dr. Reddy's | |
| ARX788 | | HER2/neu | Ambrx, Zhejiang | Trial ID: NCT04829604 |
| | | | Medicine | |
| ADCT-502 | ADC Therapeutics | HER2/neu | ADC | Trial ID: NCT03125200 |
| | patent anti-Her2 | | Therapeutics, | |
| | | | Medimmune | |
| FS102 | Fcab H10-03-6 | HER2/neu | BMS, f-star | Trial ID: NCT02286219 |
| GBR1302 | Glenmark patent anti- | CD3, | Glenmark/ | Trial ID: NCT02829372 |
| | Her2 | HER2/neu | Ichnos, Harbour | |
| | | | Biomed Ltd. | |
| M802 | | CD3, | Wuhan YZY | Yu et al., J Exp Clin |
| | | HER2/neu | Biopharma | Cancer Res. 2019 Aug. 14; |
| | | | | 38(1): 355 |
| trastuzumab- | TCMC-trastuzumab | HER2/neu | NCI | Milenic et al., |
| TCMC | | | | Pharmaceuticals (Basel). |
| | | | | 2015 Jul. 29; 8(3): 435-54 |
| zanidatamab | MEDI4276, | HER2/neu | Medimmune | Trial ID: NCT02576548 |
| zovodotin | Medimmune patent | | | |
| | anti-Her2 Bispecific | | | |
| ALT-P7 | HM2-MMAE | HER2/neu | 3SBio, Alteogen | Trial ID: NCT03281824 |
| XMT-1522 | HT-19 | HER2/neu | Adimab, Mersana, | Trial ID: NCT02952729 |
| | | | Takeda | |
| KN026 | | HER2/neu | Alphamab | Trial ID: NCT04521179 |
| Baylor | HER2-CAR | HER2/neu | Baylor Coll. Med. | Trial ID: NCT02442297 |
| Coll. Med. | | | | |
| anti-Her2 | | | | |
| CAR | | | | |
| King's College | T1E28z | HER2/neu | King's College | Trial ID: NCT01818323 |
| anti-Her2 | | | | |
| CAR | | | | |
| DMB-3111 | DMB3111 | HER2/neu | Meiji Seika | Trial ID: NCT02100917 |
| timigutuzumab | TrasGEX, | HER2/neu | Glycotope | Fiedler et al., ESMO |
| | TrastuzuMab-GEX, | | | Open. 2018 Jun. 23; |
| | GT-Mab 7.3-GEX | | | 3(4): e000381 |
| UB-921 | | HER2/neu | United | Trial ID: NCT03013881 |
| | | | Biopharma | |
| ZW49 | | HER2/neu | Zymeworks | Trial ID: NCT03821233 |
| IBI315 | IBI-315 | HER2/neu, | Beijing | Trial ID: NCT04162327 |
| | | PD-1 | Hanmi, Innovent | |
| RG6194 | BTRC4017A | HER2/neu | Genentech | Trial ID: NCT03448042 |
| RG6148 | DHES0815A | HER2/neu | Genentech | Trial ID: NCT03451162 |
| U. Virginia | Her2Bi | CD3, | U. Virginia | Trial ID: NCT03661424 |
| anti-Her2/ | | HER2/neu | | |
| CD3 | | | | |
| NJH395 | | HER2/neu | Novartis | Trial ID: NCT03696771 |
| BCD-147 | BDC-147 | HER2/neu | Biocad | Trial ID: NCT03912441 |
| B003 | | HER2/neu | Shanghai Pharma | Trial ID: NCT03953833 |
| | | | Holdings | |
| FS-1502 | Trastuzumab | HER2/neu | Fosun Pharma | Trial ID: NCT03944499 |
| | Monomethyl | | | |
| | Auristatin F | | | |
| DP303c | | HER2/neu | CSPC Pharma | Trial ID: NCT04146610 |
| BAY2701439 | BAY2701438 | HER2/neu | Bayer | Trial ID: NCT04147819 |
| BDC-1001 | | HER2/neu | Bolt Bio | Trial ID: NCT04278144 |
| SBT6050 | | HER2/neu | Silverback | Trial ID: NCT04460456 |
| B002 | | HER2/neu | Shanghai Pharma | Trial ID: NCT04382352 |
| | | | Holdings | |
| GQ1001 | | HER2/neu | GeneQuantum | Trial ID: NCT04450732 |
| NM-02 | | HER2/neu | Suzhou Nanomab | Trial ID: NCT04674722 |
| Vrije | 131 I-GMIB-Anti- | HER2/neu | Vrije Universiteit | Trial ID: NCT04467515 |
| Universiteit | HER2-VHH1 | | Brussel | |
| Brussel patent | | | | |
| anti-Her2 | | | | |
| Chinese PLA | CART-HER-2 | HER2/neu | Chinese PLA | Trial ID: NCT01935843 |
| Gen. Hosp. | | | Gen. Hosp. | |
| anti-Her2 | | | | |
| CAR | | | | |
| Sym013 | Pan-Her | EGFR, | Symphogen | Trial ID: NCT02906670 |
| | | HER2/neu, | | |
| | | HER3 | | |
| A166 | Sichuan Kelun Pharma | HER2/neu | Klus | Trial ID: NCT03602079 |
| | patent anti-Her2 | | Pharma, Sichuan | |
| | | | Kelun Pharma | |
| ertumaxomab | Rexomun, 2502A/TP- | CD3, | Fresenius | Trial ID: NCT01569412 |
| | A-02/TPBs03 | HER2/neu | | |
| MM-111 | | HER2/neu, | Merrimack | Trial ID: NCT01774851 |
| | | HER3 | | |
| zanidatamab | ZW25, ZW-25 | HER2/neu | Zymeworks | Trial ID: NCT04466891 |
| MBS301 | | HER2/neu | Beijing | Trial ID: NCT03842085 |
| | | | Mabworks | |
| zenocutuzumab | MCLA-128, MF3958, | HER2/neu, | Merus | Trial ID: NCT03321981 |
| | MF3178 | HER3 | | |
| MRG002 | | HER2/neu | Shanghai | Trial ID: NCT04742153 |
| | | | Miracogen | |
| gancotamab | MM-302, MM-001 | HER2/neu | Merrimack | Trial ID: NCT02213744 |
| trastuzumab- | Herzuma, CT-P06, | HER2/neu | Celltrion | Stebbing et al: Lancet |
| pkrb | CT-P6 | | | Oncol. 2017 July; |
| | | | | 18(7): 917-928 |
| SB3 | ONTRUZANT | HER2/neu | Merck | Trial ID: NCT03766607 |
| | | | (MSD), Samsung | |
| | | | Bioepis | |
| CMAB302 | Cipterbin, Saiputing | HER2/neu | Shanghai CP | Trial ID: NCT01291667 |
| | | | Guojian | |
| trastuzumab | SYD985, trastuzumab | HER2/neu | Synthon | Banerji et al., Lancet |
| duocarmazine | vc-seco-DUBA | | | Oncol. 2019 August; |
| | | | | 20(8): 1124-1135 |
| coprelotamab | GB221, GB235-019 | HER2/neu | Genor | Trial ID: NCT04164615 |
| BCD-022 | HERtiCAD | HER2/neu | Biocad | Trial ID: NCT01764022 |
| BAT8001 | | HER2/neu | Bio-Thera | Hong et al., Cancer |
| | | | Solutions | Commun (Lond). 2021 |
| | | | | February; 41(2): 171-182 |
| trastuzumab | ENHERTU, DS- | HER2/neu | AstraZeneca, | Shitara et al., Lancet |
| deruxtecan | 8201a, DS8201a | | Daiichi Sankyo | Oncol. 2019 June; |
| | | | | 20(6): 827-836 |
| HD201 | | HER2/neu | Prestige | Trial ID: NCT03776240 |
| | | | BioPharma | |
| HLX22 | AC101, HLX 22, | HER2/neu | Abclon, Shanghai | Trial ID: NCT03916094 |
| | 1E11 | | Henlius | |
| disitamab | Remegen RC48, | HER2/neu | RemeGen | Sheng et al., Clin Cancer |
| vedotin | RC48-ADC, | | | Res. 2021 Jan. 1; |
| | Hertuzumab-vc- | | | 27(1): 43-51 |
| | MMAE | | | |
| SIBP-01 | | HER2/neu | Shanghai | Trial ID: NCT03989037 |
| | | | Inst. Bio. | |
| | | | Products | |
| TX05 | | HER2/neu | Tanvex | Trial ID: NCT04109391 |
| QL1209 | | HER2/neu | Qilu | Trial ID: NCT04629846 |
| | | | Pharma, Sound | |
| | | | Biologics | |

In some embodiments, the HER2 targeting antibody is selected from the group consisting of trastuzumab (or a biosimilar thereof), margetuximab (or a biosimilar thereof), pertuzumab (or a biosimilar thereof), trastuzumab emtansine (or a biosimilar thereof), PF-05280014 (or a biosimilar thereof), trastuzumab-anns (or a biosimilar thereof), HLX02 (or a biosimilar thereof), trastuzumab-dkst (or a biosimilar thereof), Hervycta (or a biosimilar thereof), and combinations thereof.

In some embodiments, the HER2 targeting antibody is trastuzumab or a biosimilar thereof. In some embodiments, the HER2 targeting antibody is trastuzumab.

### V. Pharmaceutical Compositions

Provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s).

In some embodiments, the therapeutic cell(s) are animal cell(s). In some embodiments, the therapeutic cell(s) are human cell(s).

In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethyelene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

### VI. Methods of Treatment

The NK cells described herein find use for treating cancer or other proliferative disorders.

Thus, also provided herein are methods of treating a patient suffering from a disorder, e.g., a disorder associated with a cancer, e.g., a HER2+ cancer, comprising administering the NK cells, e.g., the NK cells described herein, and a HER2targeting antibody, e.g., an antibody described herein.

Also provided herein are methods of preventing, reducing and/or inhibiting the recurrence, growth, proliferation, migration and/or metastasis of a cancer cell or population of cancer cells in a subject in need thereof, comprising administering the NK cells, e.g., the NK cells described herein, and a HER2targeting antibody, e.g., an antibody described herein.

Also provided herein are methods of enhancing, improving, and/or increasing the response to an anticancer therapy in a subject in need thereof, comprising administering the NK cells, e.g., the NK cells described herein, and a HER2targeting antibody, e.g., an antibody described herein.

Also provided herein are methods for inducing the immune system in a subject in need thereof comprising administering the NK cells, e.g., the NK cells described herein, and a HER2 targeting antibody, e.g., an antibody described herein.

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. Generally, the methods include administering a therapeutically effective amount of a treatment as described herein, to a subject who is in need of, or who has been determined to be in need of, such treatment. In some embodiments, the methods include administering a therapeutically effective amount of a treatment comprising an NK cells, e.g., NK cells described herein, and a XX targeting antibody, e.g., an antibody described herein.

As used herein, the terms “treatment,” “treat,” and “treating” refer to reversing, alleviating, delaying the onset of, or inhibiting the progress of a disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, among other things. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “inhibition”, as it relates to cancer and/or cancer cell proliferation, refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of cancer is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject and/or before a certain stage of the disease (e.g., administration of a therapeutic substance to a subject with a cancer that has not yet metastasized). The subject may be an individual at risk of developing the disease or disorder, or at risk of disease progression, e.g., cancer metastasis. Such as an individual who has one or more risk factors known to be associated with development or onset of the disease or disorder. For example, an individual may have mutations associated with the development or progression of a cancer. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

### A. Disorders

Methods and manufactured compositions disclosed herein find use in targeting a number of disorders, such as cellular proliferative disorders. A benefit of the approaches herein is that allogenic cells are used in combination with exogenous antibody administration to target specific proliferating cells targeted by the exogenous antibody. Unlike previous therapies, such as chemo or radiotherapy, using the approaches and pharmaceutical compositions herein, one is able to specifically target cells exhibiting detrimental proliferative activity, potentially without administering a systemic drug or toxin that impacts proliferating cells indiscriminately.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

In some embodiments, the cancer is selected from the group consisting of: acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thymoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, and Wilms tumor.

In some embodiments, the cancer is a solid tumor.

In some embodiments, the cancer is metastatic.

In some embodiments, the cancer is a HER2+ cancer.

In some embodiments, the HER2+ cancer is selected from the group consisting of bladder cancer, breast adenocarcinoma, colorectal adenocarcinoma, non-small cell lung cancer, esophageal cancer, cervix squamous cancer, stomach adenocarcinoma, cholangiocarcinoma, ovary cancer, renal papillary cell carcinoma, and combinations thereof.

In some embodiments, the HER2+ cancer is selected from the group consisting of breast cancer, gastric cancer, and ovarian cancer.

In some embodiments, the HER2+ cancer is breast cancer. In some embodiments, the HER2+ cancer is gastric cancer. In some embodiments, the HER2+ cancer is ovarian cancer.

### B. Patients

Suitable patients for the compositions and methods herein include those who are suffering from, who have been diagnosed with, or who are suspected of having a cellular proliferative and/or differentiative disorder, e.g., a cancer. Patients subjected to technology of the disclosure herein generally respond better to the methods and compositions herein, in part because the pharmaceutical compositions are allogeneic and target cells identified by the antibodies, rather than targeting proliferating cells generally. As a result, there is less off-target impact and the patients are more likely to complete treatment regimens without substantial detrimental off-target effects.

In some embodiments, the methods of treatment provided herein may be used to treat a subject (e.g., human, monkey, dog, cat, mouse) who has been diagnosed with or is suspected of having a cellular proliferative and/or differentiative disorder, e.g., a cancer. In some embodiments, the subject is a mammal. In some embodiments, the subject is a human.

As used herein, a subject refers to a mammal, including, for example, a human.

In some embodiments, the mammal is selected from the group consisting of an armadillo, an ass, a bat, a bear, a beaver, a cat, a chimpanzee, a cow, a coyote, a deer, a dog, a dolphin, an elephant, a fox, a panda, a gibbon, a giraffe, a goat, a gopher, a hedgehog, a hippopotamus, a horse, a humpback whale, a jaguar, a kangaroo, a koala, a leopard, a lion, a llama, a lynx, a mole, a monkey, a mouse, a narwhal, an orangutan, an orca, an otter, an ox, a pig, a polar bear, a porcupine, a puma, a rabbit, a raccoon, a rat, a rhinoceros, a sheep, a squirrel, a tiger, a walrus, a weasel, a wolf, a zebra, a goat, a horse, and combinations thereof.

In some embodiments, the mammal is a human.

The subject, e.g., the human subject, can be a child, e.g., from or from about 0 to or to about 14 years in age. The subject can be a youth, e.g., from or from about 15 to or to about 24 years in age. The subject can be an adult, e.g., from or from about 25 to or to about 64 years in age. The subject can be a senior, e.g, 65+ years in age.

In some embodiments, the subject may be a human who exhibits one or more symptoms associated with a cellular proliferative and/or differentiative disorder, e.g., a cancer, e.g., a tumor. Any of the methods of treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. The methods of treatment described herein can promote reduction or retraction of a tumor, decrease or inhibit tumor growth or cancer cell proliferation, and/or induce, increase or promote tumor cell killing. In some embodiments, the subject is in cancer remission. The methods of treatment described herein can prevent or delay metastasis or recurrence of cancer.

In some embodiments, the subject is at risk, or genetically or otherwise predisposed (e.g., risk factor), to developing a cellular proliferative and/or differentiative disorder, e.g., a cancer, that has or has not been diagnosed.

As used herein, an “at risk” individual is an individual who is at risk of developing a condition to be treated, e.g., a cellular proliferative and/or differentiative disorder, e.g., a cancer. Generally, an “at risk” subject may or may not have detectable disease, and may or may not have displayed detectable disease prior to the treatment methods described herein. “At risk” denotes that an individual has one or more so-called risk factors, which are measurable parameters that correlate with development of a disease or condition and are known in the art. For example, an at risk subject may have one or more risk factors, which are measurable parameters that correlate with development of cancer. A subject having one or more of these risk factors has a higher probability of developing cancer than an individual without these risk factor(s). In general, risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

In some embodiments, the patient is diagnosed with or has been diagnosed with a HER2+ cancer.

In some embodiments, the patient is diagnosed with or has been diagnosed with a HER2+ cancer by immunohistochemical staining of a biopsy or surgical sample of the cancer. In some embodiments, the patient is or has been diagnosed with a HER2+ cancer by fluorescent in situ hybridization of a biopsy or surgical sample of the cancer.

In some embodiments, the patient is diagnosed with or has been diagnosed with a HER2+ cancer according to ASCO® Guidelines, e.g., the 2018 ASCO® Guidelines, e.g., as described in Wolff et al., “Human Epidermal Growth Factor Receptor 2 Testing in Breast Cancer,” Arch Pathol Lab Med 142:1364-82 (2018), which is hereby incorporated by reference in its entirety.

In some embodiments, the patient is diagnosed with or has been diagnosed with a HER2+ cancer by genetic analysis, e.g., by identifying a HER2 mutated cancer, e.g., a somatic mutation in the HER2 (ERBB2) gene.

In some embodiments, the patient has a cancer comprising one or more mutations set forth in Table 7, an insertion or deletion polymorphism in the HER2 gene, a copy number variation of the HER2 gene, a methylation mutation of the HER2 gene, or combinations thereof.

In some embodiments, the patient has a chromosomal translocation associated with cancer, e.g., a HER2+ cancer. In some embodiments, the patient has a fusion gene associated with cancer, e.g., a HER+ cancer.

TABLE 7
HER2 (ERBB2) Mutations (relative to Human
Genome Assembly Reference Build GRCh38.p13
(ncbi.nlm.nih.gov/assembly/88331)

| Mutation (GRCh38) | Protein Position | Consequence |
|---|---|---|
| 17:39711955:C > T | 310 | missense_variant |
| 17:39723405:G > A | 678 | missense_variant |
| 17:39723967:T > C | 755 | missense_variant |
| 17:39725079:G > A | 842 | missense_variant |
| 17:39711955:C > A | 310 | missense_variant |
| 17:39724747:G > T | 777 | missense_variant |
| 17:39724008:G > T | 769 | missense_variant |
| 17:39724004:C > G | 767 | missense_variant |
| 17:39725139:A > G | 862 | missense_variant |
| 17:39724745:G > T | 776 | missense_variant |
| 17:39723650:C > T | 733 | missense_variant |
| 17:39724008:G > C | 769 | missense_variant |
| 17:39724747:G > C | 777 | missense_variant |
| 17:39725161:T > G | 869 | missense_variant |
| 17:39710410:A > G | 277 | missense_variant |
| 17:39710414:G > A | 278 | synonymous_variant |
| 17:39711963:C > G | 313 | missense_variant |
| 17:39723966:T > C | 755 | synonymous_variant |
| 17:39724008:G > A | 769 | missense_variant |
| 17:39726610:C > T | 974 | missense_variant |
| 17:39727303:T > G | 1056 | synonymous_variant |
| 17:39727784:C > G | 1170 | missense_variant |
| 17:39706987:C > T | — | splice_region_variant |
| 17:39708375:G > A | 94 | missense_variant |
| 17:39708497:A > T | 134 | synonymous_variant |
| 17:39708530:C > T | 145 | synonymous_variant |
| 17:39709376:G > A | 166 | synonymous_variant |
| 17:39709810:C > T | — | splice_region_variant |
| 17:39710106:G > T | 222 | missense_variant |
| 17:39710164:C > T | 241 | missense_variant |
| 17:39710409:G > T | 277 | missense_variant |
| 17:39710418:G > C | 280 | missense_variant |
| 17:39712319:C > T | — | splice_region_variant |
| 17:39712429:C > T | 377 | synonymous_variant |
| 17:39715854:C > T | 476 | synonymous_variant |
| 17:39716421:G > A | 545 | missense_variant |
| 17:39716586:C > T | 573 | missense_variant |
| 17:39717397:A > C | 605 | missense_variant |
| 17:39717446:G > A | 622 | missense_variant |
| 17:39719819:C > T | 644 | missense_variant |
| 17:39719827:A > C | 647 | synonymous_variant |
| 17:39723330:C > G | 653 | missense_variant |
| 17:39723339:C > T | 656 | missense_variant |
| 17:39723530:C > T | — | splice_region_variant |
| 17:39724734:C > T | 772 | synonymous_variant |
| 17:39724902:G > A | 828 | synonymous_variant |
| 17:39725057:C > T | 834 | synonymous_variant |
| 17:39725096:C > T | 847 | synonymous_variant |
| 17:39725139:A > T | 862 | missense_variant |
| 17:39725347:G > A | 890 | synonymous_variant |
| 17:39725357:A > C | 894 | missense_variant |
| 17:39725750:C > T | 923 | synonymous_variant |
| 17:39726573:G > A | 962 | missense_variant |
| 17:39726860:C > T | 1006 | missense_variant |
| 17:39726905:G > C | 1021 | missense_variant |
| 17:39727000:C > G | 1052 | synonymous_variant |
| 17:39727847:G > A | 1191 | missense_variant |
| 17:39727973:C > T | 1233 | missense_variant |
| 17:39700256:G > T | 6 | missense_variant |
| 17:39700298:C > T | 20 | synonymous_variant |
| 17:39706995:A > T | 27 | missense_variant |
| 17:39707022:C > T | 36 | missense_variant |
| 17:39707032:C > T | 39 | missense_variant |
| 17:39707033:C > T | 39 | synonymous_variant |
| 17:39707063:C > T | 49 | synonymous_variant |
| 17:39707070:G > A | 52 | missense_variant |
| 17:39707076:C > T | 54 | stop_gained |
| 17:39707093:C > A | 59 | missense_variant |
| 17:39707114:C > A | 66 | synonymous_variant |
| 17:39708350:C > G | 85 | synonymous_variant |
| 17:39708354:G > A | 87 | missense_variant |
| 17:39708386:G > A | 97 | synonymous_variant |
| 17:39708397:T > G | 101 | missense_variant |
| 17:39708403:G > A | 103 | missense_variant |
| 17:39708406:G > A | 104 | missense_variant |
| 17:39708459:C > T | 122 | missense_variant |
| 17:39708460:C > T | 122 | missense_variant |
| 17:39708507:C > T | 138 | missense_variant |
| 17:39708508:G > A | 138 | missense_variant |
| 17:39708510:G > A | 139 | missense_variant |
| 17:39709322:C > A | 148 | synonymous_variant |
| 17:39709326:A > G | 150 | missense_variant |
| 17:39709340:G > C | 154 | missense_variant |
| 17:39709343:C > T | 155 | synonymous_variant |
| 17:39709352:C > T | 158 | synonymous_variant |
| 17:39709375:C > T | 166 | missense_variant |
| 17:39709391:C > T | 171 | synonymous_variant |
| 17:39709394:C > G | 172 | missense_variant |
| 17:39709419:C > T | 181 | missense_variant |
| 17:39709421:C > G | 181 | synonymous_variant |
| 17:39709423:C > T | 182 | missense_variant |
| 17:39709427:G > C | 183 | synonymous_variant |
| 17:39709447:G > A | 190 | missense_variant |
| 17:39709449:G > A | 191 | missense_variant |
| 17:39709824:T > C | 196 | missense_variant |
| 17:39709825:C > T | 196 | missense_variant |
| 17:39709845:C > T | 203 | missense_variant |
| 17:39709850:C > A | 204 | stop_gained |
| 17:39710087:G > T | 215 | splice_region_variant |
| 17:39710097:G > A | 219 | missense_variant |
| 17:39710099:C > A | 219 | synonymous_variant |
| 17:39710103:G > A | 221 | missense_variant |
| 17:39710111:C > A | 223 | synonymous_variant |
| 17:39710120:C > T | 226 | synonymous_variant |
| 17:39710154:G > A | 238 | missense_variant |
| 17:39710182:C > T | 247 | missense_variant |
| 17:39710184:A > G | 248 | missense_variant |
| 17:39710191:C > T | 250 | missense_variant |
| 17:39710209:C > T | — | splice_region_variant |
| 17:39710418:G > A | 280 | missense_variant |
| 17:39710433:C > T | 285 | missense_variant |
| 17:39710454:G > C | 292 | missense_variant |
| 17:39710458:C > T | 293 | missense_variant |
| 17:39710480:C > T | 300 | splice_region_variant |
| 17:39711940:C > G | 305 | missense_variant |
| 17:39711950:G > A | 308 | synonymous_variant |
| 17:39711952:G > C | 309 | missense_variant |
| 17:39711954:T > G | 310 | missense_variant |
| 17:39711962:C > G | 312 | synonymous_variant |
| 17:39711981:A > T | 319 | missense_variant |
| 17:39712006:G > A | 327 | missense_variant |
| 17:39712012:A > T | 329 | missense_variant |
| 17:39712014:C > T | 330 | missense_variant |
| 17:39712044:C > G | 340 | missense_variant |
| 17:39712323:G > T | 341 | splice_region_variant |
| 17:39712361:G > — | 354 | frameshift_variant |
| 17:39712431:G > A | 377 | synonymous_variant |
| 17:39715322:G > C | 395 | missense_variant |
| 17:39715328:C > T | 397 | synonymous_variant |
| 17:39715340:G > A | 401 | synonymous_variant |
| 17:39715344:C > T | 403 | synonymous_variant |
| 17:39715352:G > C | 405 | missense_variant |
| 17:39715445:G > T | — | splice_acceptor_variant |
| 17:39715461:C > T | 413 | missense_variant |
| 17:39715476:G > C | 418 | missense_variant |
| 17:39715478:C > T | 419 | synonymous_variant |
| 17:39715519:G > A | 432 | synonymous_variant |
| 17:39715519:G > T | 432 | synonymous_variant |
| 17:39715738:A > T | — | splice_acceptor_variant |
| 17:39715744:G > A | 440 | missense_variant |
| 17:39715766:G > C | 447 | missense_variant |
| 17:39715783:C > A | 453 | missense_variant |
| 17:39715804:G > A | 460 | missense_variant |
| 17:39715830:C > T | 468 | synonymous_variant |
| 17:39715836:T > G | 470 | missense_variant |
| 17:39715844:A > T | 473 | missense_variant |
| 17:39715867:C > — | 481 | frameshift_variant |
| 17:39716306:G > A | 507 | missense_variant |
| 17:39716354:C > T | 523 | missense_variant |
| 17:39716365:C > G | 526 | synonymous_variant |
| 17:39716377:C > T | 530 | synonymous_variant |
| 17:39716393:C > T | 536 | missense_variant |
| 17:39716401:G > A | 538 | synonymous_variant |
| 17:39716418:G > T | 544 | missense_variant |
| 17:39716552:C > T | 562 | missense_variant |
| 17:39716592:C > T | 575 | missense_variant |
| 17:39717326:G > A | 582 | missense_variant |
| 17:39717351:A > G | 590 | missense_variant |
| 17:39717358:C > T | 592 | synonymous_variant |
| 17:39717367:C > A | 595 | missense_variant |
| 17:39717376:C > T | 598 | synonymous_variant |
| 17:39717406:C > T | 608 | synonymous_variant |
| 17:39717439:G > A | 619 | synonymous_variant |
| 17:39717442:G > — | 620 | frameshift_variant |
| 17:39717466:C > G | 628 | missense_variant |
| 17:39717480:C > T | 633 | missense_variant |
| 17:39719785:A > T | — | splice_acceptor_variant |
| 17:39719786:G > C | — | splice_acceptor_variant |
| 17:39719799:G > A | 637 | synonymous_variant |
| 17:39719811:C > T | 641 | synonymous_variant |
| 17:39719820:C > T | 644 | synonymous_variant |
| 17:39719834:G > C | 649 | missense_variant |
| 17:39723323:C > G | 651 | missense_variant |
| 17:39723328:G > T | 652 | synonymous_variant |
| 17:39723334:C > A | 654 | synonymous_variant |
| 17:39723335:A > G | 655 | missense_variant |
| 17:39723350:G > C | 660 | missense_variant |
| 17:39723351:G > A | 660 | missense_variant |
| 17:39723356:C > G | 662 | missense_variant |
| 17:39723356:C > T | 662 | synonymous_variant |
| 17:39723357:T > A | 662 | missense_variant |
| 17:39723360:T > C | 663 | missense_variant |
| 17:39723368:G > T | 666 | missense_variant |
| 17:39723373:G > C | 667 | missense_variant |
| 17:39723376:G > T | 668 | synonymous_variant |
| 17:39723397:C > G | 675 | missense_variant |
| 17:39723402:G > A | 677 | missense_variant |
| 17:39723412:G > A | 680 | synonymous_variant |
| 17:39723449:G > A | 693 | missense_variant |
| 17:39723541:G > T | 697 | missense_variant |
| 17:39723562:G > A | 704 | missense_variant |
| 17:39723567:G > A | 705 | synonymous_variant |
| 17:39723577:C > G | 709 | missense_variant |
| 17:39723582:G > A | 710 | synonymous_variant |
| 17:39723594:C > T | 714 | synonymous_variant |
| 17:39723597:G > T | 715 | synonymous_variant |
| 17:39723603:G > C | 717 | missense_variant |
| 17:39723908:C > T | — | splice_region_variant |
| 17:39723909:C > T | — | splice_region_variant |
| 17:39723916:T > A | 738 | missense_variant |
| 17:39723941:G > A | 746 | synonymous_variant |
| 17:39723966:T > A | 755 | missense_variant |
| 17:39723967:T > G | 755 | missense_variant |
| 17:39723970:G > A | 756 | missense_variant |
| 17:39723974:A > C | 757 | missense_variant |
| 17:39724002:A > T | 767 | missense_variant |
| 17:39724010:C > T | 769 | splice_region_variant |
| 17:39724743:G > ATCT | 775 | inframe_insertion |
| 17:39724744:G > A | 776 | missense_variant |
| 17:39724761:T > C | 781 | synonymous_variant |
| 17:39724768:C > T | 784 | missense_variant |
| 17:39724774:C > T | 786 | synonymous_variant |
| 17:39724780:A > T | 788 | missense_variant |
| 17:39724784:G > A | 789 | missense_variant |
| 17:39724822:C > T | 802 | missense_variant |
| 17:39724874:C > T | 819 | missense_variant |
| 17:39724879:G > C | 821 | missense_variant |
| 17:39724882:C > T | 822 | synonymous_variant |
| 17:39725070:G > A | 839 | missense_variant |
| 17:39725088:G > A | 845 | missense_variant |
| 17:39725122:C > G | 856 | missense_variant |
| 17:39725172:G > A | 873 | missense_variant |
| 17:39725174:C > T | 873 | synonymous_variant |
| 17:39725177:G > A | 874 | synonymous_variant |
| 17:39725187:C > T | 878 | missense_variant |
| 17:39725192:A > G | 879 | synonymous_variant |
| 17:39725344:G > A | 889 | missense_variant |
| 17:39725363:C > T | 896 | missense_variant |
| 17:39725374:C > A | 899 | missense_variant |
| 17:39725374:C > G | 899 | missense_variant |
| 17:39725400:A > G | 908 | missense_variant |
| 17:39725726:G > C | 915 | synonymous_variant |
| 17:39725729:G > A | 916 | missense_variant |
| 17:39725756:G > A | 925 | synonymous_variant |
| 17:39725769:G > A | 930 | missense_variant |
| 17:39725797:A > G | 939 | missense_variant |
| 17:39725808:C > T | 943 | stop_gained |
| 17:39725825:C > A | 948 | synonymous_variant |
| 17:39725857:C > T | — | splice_region_variant |
| 17:39726577:C > G | 963 | missense_variant |
| 17:39726594:T > C | 969 | missense_variant |
| 17:39726596:C > G | 969 | missense_variant |
| 17:39726633:G > T | 982 | missense_variant |
| 17:39726651:G > A | 988 | missense_variant |
| 17:39726821:G > A | 993 | missense_variant |
| 17:39726829:C > T | 995 | synonymous_variant |
| 17:39726881:G > C | 1013 | missense_variant |
| 17:39726933:T > G | 1030 | missense_variant |
| 17:39726941:C > — | 1033 | frameshift_variant |
| 17:39726941:C > G | 1033 | missense_variant |
| 17:39726958:C > T | 1038 | synonymous_variant |
| 17:39726959:G > A | 1039 | missense_variant |
| 17:39726980:A > T | 1046 | missense_variant |
| 17:39726987:G > A | 1048 | missense_variant |
| 17:39727294:G > A | — | splice_acceptor_variant |
| 17:39727294:G > C | — | splice_acceptor_variant |
| 17:39727305:G > T | 1057 | missense_variant |
| 17:39727308:A > C | 1058 | missense_variant |
| 17:39727344:C > T | 1070 | missense_variant |
| 17:39727366:C > T | 1077 | synonymous_variant |
| 17:39727370:G > A | 1079 | missense_variant |
| 17:39727373:G > T | 1080 | missense_variant |
| 17:39727450:C > — | 1105 | frameshift_variant |
| 17:39727466:C > T | 1111 | missense_variant |
| 17:39727475:G > C | 1114 | missense_variant |
| 17:39727492:C > T | 1119 | synonymous_variant |
| 17:39727533:G > C | 1133 | missense_variant |
| 17:39727542:A > G | 1136 | missense_variant |
| 17:39727728:C > T | 1151 | missense_variant |
| 17:39727732:C > T | 1152 | synonymous_variant |
| 17:39727752:C > T | 1159 | missense_variant |
| 17:39727755:C > T | 1160 | missense_variant |
| 17:39727805:A > G | 1177 | missense_variant |
| 17:39727825:C > T | 1183 | synonymous_variant |
| 17:39727838:G > A | 1188 | missense_variant |
| 17:39727839:G > A | 1188 | missense_variant |
| 17:39727846:C > T | 1190 | synonymous_variant |
| 17:39727867:G > C | 1197 | missense_variant |
| 17:39727871:C > A | 1199 | missense_variant |
| 17:39727892:C > T | 1206 | stop_gained |
| 17:39727898:C > T | 1208 | missense_variant |
| 17:39727904:C > T | 1210 | missense_variant |
| 17:39727965:G > — | 1230 | frameshift_variant |
| 17:39727974:C > T | 1233 | missense_variant |
| 17:39727976:C > T | 1234 | missense_variant |
| 17:39728002:G > A | 1242 | synonymous_variant |
| 17:39728006:G > C | 1244 | missense_variant |
| 17:39728023:G > C | 1249 | synonymous_variant |
| 17:39728032:C > T | 1252 | synonymous_variant |

In some embodiments, the patient is refractory to or has a recurrence of HER2+ cancer after treatment, e.g., with trastuzumab or a biosimilar thereof.

In some embodiments, the patient is refractory to or has a recurrence after treatment with pertuxumab (or FDA-approved biosimilar thereof), trastuzumab (or FDA-approved biosimilar thereof) and docetaxel (or pharmaceutically acceptable salt thereof). In some embodiments, the pertuzumab (or FDA-approved biosimilar thereof) is administered at 840 mg IV day 1 followed by 420 mg IV. In some embodiments, the trastuzumab (or FDA-approved biosimilar thereof) is administered at 7 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration. In some embodiments, the docetaxel (or pharmaceutically acceptable salt thereof) is administered at 75-100 mg/m2 IV day 1 cycled every 21 days.

In some embodiments, the patient is refractory to or has a recurrence after treatment with pertuxumab (or FDA-approved biosimilar thereof), trastuzumab (or FDA-approved biosimilar thereof), and paclitaxel (or pharmaceutically acceptable salt thereof). In some embodiments, the pertuxumab (or FDA-approved biosimilar thereof) is administered at 840 mg IV day 1 followed by 420 mg IV, cycled every 21 days. In some embodiments, the trastuzumab (or FDA-approved biosimilar thereof) is administered at 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration. In some embodiments, the paclitaxel (or pharmaceutically acceptable salt thereof) is administered at 80 mg/m2 IV day 1 weekly or 175 mg/m2 day 1 cycled every 21 days.

In some embodiments, the patient is refractory to or has a recurrence after treatment with tucatinib (or pharmaceutically acceptable salt thereof), trastuzumab (or FDA-approved biosimilar thereof), and capecitabine (or pharmaceutically acceptable salt thereof). In some embodiments, the tucatinib (or FDA-approved biosimilar thereof) is administered at 300 mg orally twice daily on days 1-21. In some embodiments, the trastuzumab (or FDA-approved biosimilar thereof) is administered at 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration. In some embodiments, the capecitabine (or FDA-approved biosimilar thereof) is administered at 1000 mg/m2 orally twice daily on days 1-14. In some embodiments, the administration of tucatinib (or FDA-approved biosimilar thereof), trastuzumab (or FDA-approved biosimilar thereof), and capecitabine (or pharmaceutically acceptable salt thereof) is cycled every 21 days.

In some embodiments, the patient is refractory to or has a recurrence after treatment with ado-trastuzumab emtansine (T-DM1) (or FDA-approved biosimilar thereof). In some embodiments, the ado-trastuzumab emtansine (T-DM1) (or FDA-approved biosimilar thereof) is administered at 3.6 mg/kg IV day 1, cycled every 21 days.

In some embodiments, the patient is refractory to or has a recurrence after treatment with fam-trastuzumab deruxtecan-nxki (or FDA-approved biosimilar thereof). In some embodiments, the fam-trastuzumab deruxtecan-nxki (or FDA-approved biosimilar thereof) is administered at 5.4 mg/kg IV day 1, cycled every 21 days.

In some embodiments, the patient is refractory to or has a recurrence after treatment with paclitaxel/carboplatin (or pharmaceutically acceptable salts thereof) and trastuxumab (or FDA-approved biosimilar thereof). In some embodiments, the carboplatin/paclitaxel (or pharmaceutically acceptable salts thereof) is administered at AUC 6 IV day 1 carboplatin and 175 mg/m2 IV day 1 paclitaxel), cycled every 21 days. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with paclitaxel/carboplatin (or pharmaceutically acceptable salts thereof) and trastuxumab (or FDA-approved biosimilar thereof). In some embodiments, the carboplatin/paclitaxel (or pharmaceutically acceptable salts thereof) is administered at AUC 2 IV carboplatin and 80 mg/m2 IV day 1 paclitaxel), days 1, 8, and 15, cycled every 28 days. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and paclitaxel (or pharmaceutically acceptable salt thereof). In some embodiments, the paclitaxel (or pharmaceutically acceptable salt thereof) is administered at 175 mg/m2 IV day 1 cycled every 21 days or 80-90 mg/m2 IV day 1 weekly. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and docetaxel (or pharmaceutically acceptable salt thereof). In some embodiments, the docetaxel (or pharmaceutically acceptable salt thereof) is administered at 80-100 mg/m2 IV day 1 cycled every 21 days or 35 mg/m2 IV days 1, 8, and 15 weekly. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and vinorelbine (or pharmaceutically acceptable salt thereof). In some embodiments, the vinorelbine (or pharmaceutically acceptable salt thereof) is administered at 25 mg/m2 IV day 1 weekly or 20-35 mg/m2 IV days 1 and 8, cycled every 21 days, or 25-30 mg/m2 IV days 1, 8, and 15, cycled every 28 days. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with trastuzumab (or FDA-approved biosimilar thereof) and capecitabine (or pharmaceutically acceptable salt thereof). In some embodiments, the capecitabine (or pharmaceutically acceptable salt thereof) is administered at 1000-1250 mg/m2 PO twice daily days 1-14 cycled every 21 days. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with lapatinib (or pharmaceutically acceptable salt thereof) and capecitabine (or pharmaceutically acceptable salt thereof). In some embodiments, the lapatinib (or pharmaceutically acceptable salt thereof) is administered at 1250 mg/m2 PO daily days 1-21. In some embodiments, the capecitabine (or pharmaceutically acceptable salt thereof) is administered at 1000 mg/m2 PO twice daily days 1-14, cycled every 21 days.

In some embodiments, the patient is refractory to or has a recurrence after treatment with trastuzumab (or FDA-approved biosimilar thereof) and lapatinib (or pharmaceutically acceptable salt thereof). In some embodiments, the administered (or pharmaceutically acceptable salt thereof) is administered at 1000 mg/m2 PO daily. In some embodiments, the trastuzumab (or FDA-approved biosimilar thereof) is administered 4 mg/kg IV day 1 followed by 2 mg/kg IV weekly or 8 mg/kg IV day 1 followed by 6 mg/kg IV day 1 every 21 days. In some embodiments, the trastuxumab (or FDA-approved biosimilar thereof) is administered as a trastuzumab (or FDA-approved biosimilar thereof) and hyaluronidase-oysk injection for subcutaneous administration.

In some embodiments, the patient is refractory to or has a recurrence after treatment with neratinib (or pharmaceutically acceptable salt thereof) and capecitabine (or pharmaceutically acceptable salt thereof). In some embodiments, the neratinib is administered at 240 mg/m2 PO daily on days 1-21. In some embodiments, the capecitabine is administered at 750 mg/m2 PO twice daily on days 1-14, cycled every 21 days.

### C. Lymphodepletion

In some embodiments, the patient is lymphodepleted before treatment.

Illustrative lymphodepleting chemotherapy regimens, along with correlative beneficial biomarkers, are described in WO 2016/191756 and WO 2019/079564, hereby incorporated by reference in their entirety. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day).

In some embodiments, lymphodepletion comprises administration of or of about 250 to about 500 mg/m² of cyclophosphamide, e.g., from or from about 250 to or to about 500, 250, 400, 500, about 250, about 400, or about 500 mg/m² of cyclophosphamide.

In some embodiments, lymphodepletion comprises administration of or of about 20 mg/m²/day to or to about 40 mg/m²/day fludarabine, e.g., 30 or about 30 mg/m²/day.

In some embodiments, lymphodepletion comprises administration of both cyclophosmamide and fludarabine.

In some embodiments, the patient is lymphodepleted by intravenous administration of cyclophosphamide (250 mg/m²/day) and fludarabine (30 mg/m²/day).

In some embodiments, the patient is lymphodepleted by intravenous administration of cyclophosphamide (500 mg/m²/day) and fludarabine (30 mg/m²/day).

In some embodiments, the lymphodepletion occurs no more than 5 days prior to the first dose of NK cells. In some embodiments, the lymphodepletion occurs no more than 7 days prior to the first dose of NK cells.

In some embodiments, lymphodepletion occurs daily for 3 consecutive days, starting 5 days before the first dose of NK cells (i.e., from Day −5 through Day −3).

In some embodiments, the lymphodepletion occurs on day −5, day −4 and day −3.

### D. Administration

### 1. NK Cells

In some embodiments, the NK cells are administered as part of a pharmaceutical composition, e.g., a pharmaceutical composition described herein. Cells are administered after thawing, in some cases without any further manipulation in cases where their cryoprotectant is compatible for immediate administration. For a given individual, a treatment regimen often comprises administration over time of multiple aliquots or doses of NK cells drawn from a common batch or donor.

In some embodiments, the NK cells, e.g., the NK cells described herein are administered at or at about 1×10⁸ to or to about 8×10⁹ NK cells per dose. In some embodiments, the NK cells are administered at or at about 1×10⁸, at or at about 1×10⁹, at or at about 4×10⁹, or at or at about 8×10⁹ NK cells per dose.

In some embodiments, the NK cells are administered weekly. In some embodiments, the NK cells are administered for or for about weeks. In some embodiments, the NK cells are administered weekly for or for about 8 weeks.

In some embodiments, the NK cells are cryopreserved in an infusion-ready media, e.g., a cryopreservation composition suitable for intravenous administration, e.g., as described herein.

In some embodiments, the NK cells are cryopreserved in vials containing from or from about 1×10⁸ to or to about 8×10⁹ cells per vial. In some embodiments, the NK cells are cryopreserved in vials containing a single dose.

In some embodiments, the cells are thawed, e.g., in a 37° C. water bath, prior to administration.

In some embodiments, the thawed vial(s) of NK cells are aseptically transferred to a single administration vessel, e.g., administration bag using, e.g., a vial adapter and a sterile syringe. The NK cells can be administered to the patient from the vessel through a Y-type blood/solution set filter as an IV infusion, by gravity.

In some embodiments, the NK cells are administered as soon as practical, preferably less than 90 minutes, e.g., less than 80, 70, 60, 50, 40, 30, 20, or 10 minutes after thawing. In some embodiments, the NK cells are administered within 30 minutes of thawing.

In some embodiments, the pharmaceutical composition is administered intravenously via syringe.

In some embodiments, 1 mL, 4 mL, or 10 mL of drug product is administered to the patient intravenously via syringe.

### 2. Antibodies

In some embodiments, the NK cell(s) described herein, e.g., the pharmaceutical compositions comprising NK cell(s) described herein, are administered in combination with an antibody, e.g., an antibody described herein, e.g., a HER2 antibody. In some embodiments, an antibody is administered together with the NK cells as part of a pharmaceutical composition. In some embodiments, an antibody is administered separately from the NK cells, e.g., as part of a separate pharmaceutical composition. Antibodies can be administered prior to, subsequent to, or simultaneously with administration of the NK cells.

In some embodiments, the antibody is administered before the NK cells. In some embodiments, the antibody is administered after the NK cells.

In some embodiments, the NK cells are administered at least 30 minutes, 60 minutes, 90 minutes, 120 minutes, 150 minutes, 180 minutes, 210 minutes, or 240 minutes after completing administration of the antibody.

In some embodiments, the NK cells are administered the day after the antibody is administered.

In some embodiments, the NK cells are administered at each administration, while the antibody is administered at a subset of the administrations. For example, in some embodiments, the NK cells are administered once a week and the antibody is administered once a month.

In some embodiments, the antibody is administered weekly for 8 weeks. In some embodiments, the antibody is administered every two weeks for 8 weeks.

In some embodiments, a dose of antibody is given prior to the first dose of cells. In some embodiments, a debulking dose of the antibody is given prior to the first dose of cells.

### 3. Cytokines

In some embodiments, a cytokine is administered to the patient.

In some embodiments, the cytokine is administered together with the NK cells as part of a pharmaceutical composition. In some embodiments, the cytokine is administered separately from the NK cells, e.g., as part of a separate pharmaceutical composition.

In some embodiments, the cytokine is IL-2.

In some embodiments, the IL-2 is administered subcutaneously.

In some embodiments, the IL-2 is administered from between 1 to 4 or about 1 to about 4 hours following the conclusion of NK cell administration. In some embodiments, the IL-2 is administered at least 1 hour following the conclusion of NK cell administration. In some embodiments, the IL-2 is administered no more than 4 hours following the conclusion of NK cell administration. In some embodiments, the IL-2 is administered at least 1 hour after and no more than 4 hours following the conclusion of NK cell administration.

In some embodiments, the IL-2 is administered at up to 10 million IU/M², e.g., up to 1 million, 2 million, 3 million, 4 million, 5 million, 6 million, 7 million, 8 million, 9 million, or 10 million IU/m².

In some embodiments, the IL-2 is administered at or at about 1 million, at or at about 2 million, at or at about 3 million, at or at about 4 million, at or at about 5 million, at or at about 6 million, at or at about 7 million, at or at about 8 million, at or at about 9 million, at or at about 10 million IU/M²

In some embodiments, the IL-2 is administered at or at about 1×10⁶ IU/M². In some embodiments, the IL-2 is administered at or at about 2×10⁶ IU/M².

In some embodiments, less than 1×10⁶ IU/M²IL-2 is administered to the patient.

In some embodiments, a flat dose of IL-2 is administered to the patient. In some embodiments, a flat dose of 6 million IU or about 6 million IU is administered to the patient.

In some embodiments, IL-2 is not administered to the patient.

### E. Dosing

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### F. Combination Therapies

In some embodiments, the method comprises administering the NK cells described herein and a HER2 targeted antibody in combination with another therapy, e.g., an additional antibody, an NK cell engager, an antibody drug conjugate (ADC), a chemotherapy drug, e.g., a small molecule drug, an immune checkpoint inhibitor, and combinations thereof.

### 1. Small Molecule/Chemotherapy Drugs

In some embodiments, the additional therapy is a small molecule drug. In some embodiments, the additional therapy is a chemotherapy drug. In some embodiments, the additional therapy is a small molecule chemotherapy drug. Such small molecule drugs can include existing standard-of-care treatment regimens to which adoptive NK cell therapy is added. In some cases, the use of the NK cells described herein can enhance the effects of small molecule drugs, including by enhancing the efficacy, reducing the amount of small molecule drug necessary to achieve a desired effect, or reducing the toxicity of the small molecule drug.

In some embodiments, the drug is selected from the group consisting of

In some embodiments, the drug is [(1S,2S,3R,4S,7R,9S,10S,12R,15S)-4-acetyloxy-1,9,12-trihydroxy-15-[(2R,3S)-2-hydroxy-3-[(2-methylpropan-2-yl)oxycarbonylamino]-3-phenylpropanoyl]oxy-10,14,17,17-tetramethyl-11-oxo-6-oxatetracyclo[11.3.1.0^{3,10}.0^{4,7}]heptadec-13-en-2-yl] benzoate (docetaxel) or a pharmaceutically acceptable salt thereof.

In some embodiments, the drug is [(1S,2S,3R,4S,7R,9S,10S,12R,15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.0^{3,10}.0^{4,7}]heptadec-13-en-2-yl] benzoate (paclitaxel) or a pharmaceutically acceptable salt thereof.

In some embodiments, the drug is 6-N-(4,4-dimethyl-5H-1,3-oxazol-2-yl)-4-N-[3-methyl-4-([1,2,4]triazolo[1,5-a]pyridin-7-yloxy)phenyl]quinazoline-4,6-diamine (tucatinib) or a pharmaceutically acceptable salt thereof.

In some embodiments, the drug is pentyl N-[1-[(2R,3R,4S,5R)-3,4-dihydroxy-5-methyloxolan-2-yl]-5-fluoro-2-oxopyrimidin-4-yl]carbamate (capecitabine) or a pharmaceutically acceptable salt thereof.

In some embodiments, the drug is azanide; cyclobutane-1,1-dicarboxylic acid; platinum(2+) (carboplatin) or a pharmaceutically acceptable salt thereof.

In some embodiments, the drug is methyl (1R,9R,10S,11R,12R,19R)-11-acetyloxy-12-ethyl-4-[(12S,14R)-16-ethyl-12-methoxycarbonyl-1,10-diazatetracyclo[12.3.1.0^{3,11}.0^{4,9}]octadeca-3(11),4,6,8,15-pentaen-12-yl]-10-hydroxy-5-methoxy-8-methyl-8,16-diazapentacyclo[10.6.1.0^{1,9}.0^{2,7}.0^{16,19}]nonadeca-2,4,6,13-tetraene-10-carboxylate (vinorelbine) or a pharmaceutically acceptable salt thereof.

In some embodiments, the drug is N-[3-chloro-4-[(3-fluorophenyl)methoxy]phenyl]-6-[5-[(2-methylsulfonylethylamino)methyl]furan-2-yl]quinazolin-4-amine (lapatinib) or a pharmaceutically acceptable salt thereof.

In some embodiments, the drug is (E)-N-[4-[3-chloro-4-(pyridin-2-ylmethoxy)anilino]-3-cyano-7-ethoxyquinolin-6-yl]-4-(dimethylamino)but-2-enamide (neratinib) or a pharmaceutically acceptable salt thereof.

In some embodiments, the drug is 6-acetyl-8-cyclopentyl-5-methyl-2-[(5-piperazin-1-ylpyridin-2-yl)amino]pyrido[2,3-d]pyrimidin-7-one (palbociclib) or a pharmaceutically acceptable salt thereof.

In some embodiments, the drug is 7-cyclopentyl-N,N-dimethyl-2-[(5-piperazin-1-ylpyridin-2-yl)amino]pyrrolo[2,3-d]pyrimidine-6-carboxamide (ribociclib) or a pharmaceutically acceptable salt thereof.

In some embodiments, the drug is N-[5-[(4-ethylpiperazin-1-yl)methyl]pyridin-2-yl]-5-fluoro-4-(7-fluoro-2-methyl-3-propan-2-ylbenzimidazol-5-yl)pyrimidin-2-amine (abemaciclib) or a pharmaceutically acceptable salt thereof.

In some embodiments, the drug is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone (everolimus) or a pharmaceutically acceptable salt thereof.

In some embodiments, the drug is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide (alpelisib) or a pharmaceutically acceptable salt thereof.

In some embodiments, the drug is 4-[[3-[4-(cyclopropanecarbonyl)piperazine-1-carbonyl]-4-fluorophenyl]methyl]-2H-phthalazin-1-one (olaparib) or a pharmaceutically acceptable salt thereof.

In some embodiments, the drug is (11S,12R)-7-fluoro-11-(4-fluorophenyl)-12-(2-methyl-1,2,4-triazol-3-yl)-2,3,10-triazatricyclo[7.3.1.0^{5,13}]trideca-1,5(13),6,8-tetraen-4-one (talazoparib) or a pharmaceutically acceptable salt thereof.

In some embodiments, the drug is N-[2-[2-(dimethylamino)ethyl-methylamino]-4-methoxy-5-[[4-(1-methylindol-3-yl)pyrimidin-2-yl]amino]phenyl]prop-2-enamid (osimertinib) or a pharmaceutically acceptable salt thereof.

In some embodiments, the drug is N-(3-chloro-4-fluorophenyl)-7-methoxy-6-(3-morpholin-4-ylpropoxy)quinazolin-4-amine (gefitinib) or a pharmaceutically acceptable salt thereof.

In some embodiments, the drug is N-(3-ethynylphenyl)-6,7-bis(2-methoxyethoxy)quinazolini-4-anine (erlotinib) or a pharmaceutically acceptable salt thereof.

In some embodiments, the drug is (E)-N-[4-(3-chloro-4-fluoroanilino)-7-[(3S)-oxolan-3-yl]oxyquinazolin-6-yl]-4-(dimethylamino)but-2-enamide (afatinib) or a pharmaceutically acceptable salt thereof.

In some embodiments, the drug is azane; dichloroplatinum (cisplatin, platinol) or a pharmaceutically acceptable salt thereof.

In some embodiments, the drug is azanide; cyclobutane-1,1-dicarboxylic acid; platinum(2+) (carboplatin) or a pharmaceutically acceptable salt thereof

In some embodiments, the drug is 4-amino-1-[(2R,4R,5R)-3,3-difluoro-4-hydroxy-5-(hydroxymethyl)oxolan-2-yl]pyrimidin-2-one (gemcitabine) or a pharmaceutically acceptable salt thereof.

In some embodiments, the drug is (2S)-2-[[4-[2-(2-amino-4-oxo-3,7-dihydropyrrolo[2,3-d]pyrimidin-5-yl)ethyl]benzoyl]amino]pentanedioic acid (pemetrexed) or a pharmaceutically acceptable salt thereof.

In some embodiments, the drug is N,N-bis(2-chloroethyl)-2-oxo-1,3,2k-oxazaphosphinan-2-amine (cyclophosphamide) or a pharmaceutically acceptable salt thereof.

In some embodiments, the drug is (2R,3S,4S,5R)-2-(6-amino-2-fluoropurin-9-yl)-5-(hydroxymethyl)oxolane-3,4-diol (fludarabine) or a pharmaceutically acceptable salt thereof.

In some embodiments, the drug is (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (doxorubicin) or a pharmaceutically acceptable salt thereof.

In some embodiments, the drug is methyl (1R,9R,10S,11R,12R,19R)-11-acetyloxy-12-ethyl-4-[(13S,15S,17S)-17-ethyl-17-hydroxy-13-methoxycarbonyl-1,11-diazatetracyclo[13.3.1.0^{4,12}.0^{5,10}]nonadeca-4(12),5,7,9-tetraen-13-yl]-8-formyl-10-hydroxy-5-methoxy-8,16-diazapentacyclo[10.6.1.0^{1,9}.0^{2,7}.0^{16,19}]nonadeca-2,4,6,13-tetraene-10-carboxylate (vincristine) or a pharmaceutically acceptable salt thereof.

In some embodiments, the drug is (8S,9S,10R,13S,14S,17R)-17-hydroxy-17-(2-hydroxyacetyl)-10,13-dimethyl-6,7,8,9,12,14,15,16-octahydrocyclopenta[a]phenanthrene-3,11-dione (prednisone) or a pharmaceutically acceptable salt thereof.

In some embodiments, the drug is N,3-bis(2-chloroethyl)-2-oxo-1,3,2λ⁵-oxazaphosphinan-2-amine (ifosfamide) or a pharmaceutically acceptable salt thereof.

In some embodiments, the drug is (5S,5aR,8aR,9R)-5-[[(2R,4aR,6R,7R,8R,8aS)-7,8-dihydroxy-2-methyl-4,4a,6,7,8,8a-hexahydropyrano[3,2-d][1,3]dioxin-6-yl]oxy]-9-(4-hydroxy-3,5-dimethoxyphenyl)-5a,6,8a,9-tetrahydro-5H-[2]benzofuro[6,5-f][1,3]benzodioxol-8-one (etopside) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (dexamethasone) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (cytarabine) or a pharmaceutically acceptable salt thereof.

In some embodiments, the NK cells, e.g., the NK cells described herein, e.g., AB-101 cells, are administered in combination with a HER2 targeting antibody as well as a therapy selected from the group consisting of an antibody-drug conjugate (ADC), a kinase inhibitor, a CDK4/5 inhibitor, an mTOR inhibitor, a PI3K inhibitor, a PARP inhibitor, or a combination thereof.

In some embodiments, the antibody-drug conjugate is selected from the group consisting of ado-trastuxumab emtansine, fam-trastuzumab deruxtecan, sacituzumab govitecan, and combinations thereof.

In some embodiments, the kinase inhibitor is selected from the group consisting of lapatinib, neratinib, tucatinib, and combinations thereof.

In some embodiments, the CDK4/6 inhibitor is selected from the group consisting of palbociclib, ribociclib, abemaciclib, and combinations thereof.

In some embodiments, the mTOR inhibitor is everolimus.

In some embodiments, the PI3K inhibitor is alpelisib.

In some embodiments, the PARP inhibitor is selected from the group consisting of olaparib, talazoparib, and combinations thereof.

### 2. NK Cell Engagers

In some embodiments, the additional therapy is an NK cell engager, e.g., a bispecific or trispecific antibody.

In some embodiments, the NK cell engager is a bispecific antibody against CD16 and a disease-associated antigen, e.g., cancer-associated antigen, e.g., an antigen of cancers described herein, e.g., HER2. In some embodiments, the NK cell engager is a trispecific antibody against CD16 and two disease-associated antigens, e.g., cancer-associated antigens, e.g., antigens of cancers described herein.

### 3. Checkpoint Inhibitors

In some embodiments, the additional therapy is an immune checkpoint inhibitor.

In some embodiments, the immune checkpoint inhibitor is selected from the group consisting of a PD-1 inhibitor, a PD-L1 inhibitor, a CTLA-4 inhibitor, and combinations thereof.

In some embodiments, the immune checkpoint inhibitor is selected from the group consisting of a PD-1 inhibitor, a PD-L1 inhibitor, a CTLA-4 inhibitor, a VISTA inhibitor, a BTLA inhibitor, a TIM-3 inhibitor, a KIR inhibitor, a LAG-3 inhibitor, a TIGIT inhibitor, a CD-96 inhibitor, a SIRPα inhibitor, and combinations thereof.

In some embodiments, the immune checkpoint inhibitor is selected from the group consisting of a PD-1 inhibitor, a PD-L1 inhibitor, a CTLA-4 inhibitor, a LAG-3 (CD223) inhibitor, a TIM-3 inhibitor, a B7-H3 inhibitor, a B7-H4 inhibitor, an A2aR inhibitor, a CD73 inhibitor, a NKG2A inhibitor, a PVRIG/PVRL2 inhibitor, a CEACAM1 inhibitor, a CEACAM 5 inhibitor, a CEACAM 6 inhibitor, a FAK inhibitor, a CCL2 inhibitor, a CCR2 inhibitor, a LIF inhibitor, a CD47 inhibitor, a SIRPα inhibitor, a CSF-1 inhibitor, an M-CSF inhibitor, a CSF-1R inhibitor, an IL-1 inhibitor, an IL-1R3 inhibitor, an IL-RAP inhibitor, an IL-8 inhibitor, a SEMA4D inhibitor, an Ang-2 inhibitor, a CELVER-1 inhibitor, an Axl inhibitor, a phsphatidylserine inhibitor, and combinations thereof.

In some embodiments, the immune checkpoint inhibitor is selected from those shown in Table 8, or combinations thereof.

TABLE 8
Exemplary Immune Checkpoint Inhibitors

| Target | Inhibitor |
|---|---|
| LAG-3 (CD223) | LAG525 (IMP701), REGN3767 |
| | (R3767), BI 754,091, tebotelimab |
| | (MGD013), eftilagimod alpha |
| | (IMP321), FS118 |
| TIM-3 | MBG453, Sym023, TSR-022 |
| B7-H3, B7-H4 | MGC018, FPA150 |
| A2aR | EOS100850, AB928 |
| CD73 | CPI-006 |
| NKG2A | Monalizumab |
| PVRIG/PVRL2 | COM701 |
| CEACAM1 | CM24 |
| CEACAM 5/6 | NEO-201 |
| FAK | Defactinib |
| CCL2/CCR2 | PF-04136309 |
| LIF | MSC-1 |
| CD47/SIRPα | Hu5F9-G4 (5F9), ALX148, TTI-662, RRx-001 |
| CSF-1 | Lacnotuzumab (MCS110), LY3022855, |
| (M-CSF)/CSF-1R | SNDX-6352, emactuzumab |
| | (RG7155), pexidartinib (PLX3397) |
| IL-1 and IL-1R3 | CAN04, Canakinumab (ACZ885) |
| (IL-1RAP) | |
| IL-8 | BMS-986253 |
| SEMA4D | Pepinemab (VX15/2503) |
| Ang-2 | Trebananib |
| CLEVER-1 | FP-1305 |
| Ax1 | Enapotamab vedotin (EnaV) |
| Phosphatidylserine | Bavituximab |

In some embodiments, the immune checkpoint inhibitor is an antibody.

In some embodiments, the PD-1 inhibitor is selected from the group consisting of pembrolizumab, nivolumab, toripalimab, cemiplimab-rwlc, sintilimab, and combinations thereof

In some embodiments, the PD-L1 inhibitor is selected from the group consisting of atezolizumab, durvalumab, avelumab, and combinations thereof

In some embodiments, the CTLA-4 inhibitor is ipilimumab.

In some embodiments, the PD-1 inhibitor is selected from the group of inhibitors shown in Table 9.

TABLE 9
Exemplary PD-1 Inhibitor Antibodies

| Name | Internal Name | Antigen | Company |
|---|---|---|---|
| nivolumab | Opdivo, ONO-4538, MDX- | PD-1 | BMS, Medarex, Ono |
| | 1106, BMS-936558, 5C4 | | |
| pembrolizumab | Keytruda, MK-3475, SCH | PD-1 | Merck (MSD), Schering-Plough |
| | 900475, lambrolizumab | | |
| toripalimab | JS001, JS-001, TAB001, | PD-1 | Junmeng Biosciences, Shanghai |
| | Triprizumab | | Junshi, TopAlliance Bio |
| cemiplimab-rwlc | Libtayo, cemiplimab, | PD-1 | Regeneron, Sanofi |
| | REGN2810 | | |
| sintilimab | Tyvyt, IBI308 | PD-1 | Adimab, Innovent, Lilly |
| MEDI0680 | AMP-514 | PD-1 | Amplimmune, Medimmune |
| LZM009 | | PD-1 | Livzon |
| vudalimab | XmAb20717 | CTLA4, | Xencor |
| | | PD-1 | |
| SI-B003 | | CTLA4, | Sichuan Baili |
| | | PD-1 | Pharma, Systimmune |
| Sym021 | Symphogen patent | PD-1 | Symphogen |
| LVGN3616 | anti-PD-1 | PD-1 | Lyvgen Biopharma |
| MGD019 | | CTLA4, | MacroGenics |
| | | PD-1 | |
| MEDI5752 | | CTLA4, | Medimmune |
| | | PD-1 | |
| CS1003 | | PD-1 | CStone Pharma |
| IBI319 | IBI-319 | PD-1, | Innovent, Lilly |
| | | Undisclosed | |
| IBI315 | IBI-315 | HER2/neu, | Beijing Hanmi, Innovent |
| | | PD-1 | |
| budigalimab | ABBV-181, PR-1648817 | PD-1 | Abbvie |
| Sunshine Guojian | 609A | PD-1 | Sunshine Guojian Pharma |
| patent anti-PD-1 | | | |
| F520 | | PD-1 | Shandong New Time Pharma |
| RO7247669 | | LAG-3, PD-1 | Roche |
| izuralimab | XmAb23104 | ICOS, PD-1 | Xencor |
| LY3434172 | | PD-1, PD-L1 | Lilly, Zymeworks |
| SG001 | | PD-1 | CSPC Pharma |
| QL1706 | PSB205 | CTLA4, | Sound Biologics |
| | | PD-1 | |
| AMG 404 | AMG404 | PD-1 | Amgen |
| MW11 | | PD-1 | Mabwell |
| GNR-051 | | PD-1 | IBC Generium |
| Ningbo Cancer | HerinCAR-PD1 | PD-1 | Ningbo Cancer Hosp. |
| Hosp. anti-PD-1 | | | |
| CAR | | | |
| Chinese PLA | | PD-1 | Chinese PLA Gen.Hosp. |
| Gen. Hosp. anti- | | | |
| PD-1 | | | |
| cetrelimab | JNJ-63723283 | PD-1 | Janssen Biotech |
| TY101 | | PD-1 | Tayu Huaxia |
| AK112 | | PD-1, VEGF | Akeso |
| EMB-02 | | LAG-3, PD-1 | EpimAb |
| pidilizumab | CT-011, hBat-1, MDV9300 | PD-1 | CureTech, Medivation, Teva |
| sasanlimab | PF-06801591, RN-888 | PD-1 | Pfizer |
| balstilimab | AGEN2034, AGEN-2034 | PD-1 | Agenus, Ludwig Inst., Sloan- |
| | | | Kettering |
| geptanolimab | CBT-501, GB226, GB 226, | PD-1 | CBT Pharma, Genor |
| | Genolimzumab, Genormab | | |
| RO7121661 | | PD-1, TIM-3 | Roche |
| AK104 | | CTLA4, | Akeso |
| | | PD-1 | |
| pimivalimab | JTX-4014 | PD-1 | Jounce |
| IBI318 | IBI-318 | PD-1, PD-L1 | Innovent, Lilly |
| BAT1306 | | PD-1 | Bio-Thera Solutions |
| ezabenlimab | BI754091, BI 754091 | PD-1 | Boehringer |
| Henan Cancer | Teripalimab | PD-1 | Henan Cancer Hospital |
| Hospital anti-PD-1 | | | |
| tebotelimab | | LAG-3, PD-1 | MacroGenics |
| sindelizumab | | PD-1 | Nanjing Medical U. |
| dostarlimab | ANB011, TSR-042, ABT1 | PD-1 | AnaptysBio, Tesaro |
| tislelizumab | BGB-A317 | PD-1 | BeiGene, Celgene |
| spartalizumab | PDR001, BAP049 | PD-1 | Dana-Farber, Novartis |
| retifanlimab | MGA012, INCMGA00012 | PD-1 | Incyte, MacroGenics |
| camrelizumab | SHR-1210 | PD-1 | Incyte, Jiangsu |
| | | | Hengrui, Shanghai Hengrui |
| zimberelimab | WBP3055, GLS-010, | PD-1 | Arcus, Guangzhou Gloria |
| | AB122 | | Bio, Harbin Gloria |
| | | | Pharma, WuXi Biologics |
| penpulimab | AK105 | PD-1 | Akeso, HanX Bio, Taizhou |
| | | | Hanzhong Bio |
| prolgolimab | BCD-100 | PD-1 | Biocad |
| HX008 | | PD-1 | Taizhou Hanzhong Bio, Taizhou |
| | | | Houde Aoke Bio |
| SCT-I10A | | PD-1 | Sinocelltech |
| serplulimab | HLX10 | PD-1 | Henlix |

In some embodiments, the PD-L1 inhibitor is selected from the group of inhibitors shown in Table 10.

TABLE 10
Exemplary PD-L1 Inhibitor Antibodies

| Name | Internal Name | Antigen | Company |
|---|---|---|---|
| durvalumab | Imfinzi, | PD-L1 | AstraZeneca, Celgene, |
| | MEDI-4736, | | Medimmune |
| | MEDI4736 | | |
| atezolizumab | Tecentriq, | PD-L1 | Genentech |
| | MPDL3280A, | | |
| | RG7446, | | |
| | YW243.55.S70, | | |
| | RO5541267 | | |
| avelumab | Bavencio, | PD-L1 | Merck Serono, Pfizer |
| | MSB0010718C, | | |
| | A09-246-2 | | |
| AMP-224 | | PD-L1 | Amplimmune, GSK, |
| | | | Medimmune |
| cosibelimab | CK-301, | PD-L1 | Checkpoint Therapeutics, |
| | TG-1501 | | Dana-Farber, |
| | | | Novartis, TG |
| | | | Therapeutics |
| lodapolimab | LY3300054 | PD-L1 | Lilly |
| MCLA-145 | | 4-1BB, | Merus |
| | | PD-L1 | |
| FS118 | | LAG-3, | f-star, Merck Serono |
| | | PD-L1 | |
| INBRX-105 | ES101 | 4-1BB, | Elpiscience, Inhibrx |
| | | PD-L1 | |
| Suzhou | | PD-L1 | Suzhou Nanomab |
| Nanomab | | | |
| patent | | | |
| anti-PD-L1 | | | |
| MSB2311 | | PD-L1 | Mabspace |
| BCD-13 | | PD-L1 | Biocad |
| opucolimab | HLX20, | PD-L1 | Henlix |
| IBI322 | HLX09 | CD47, | Innovent |
| | IBI-322 | PD-L1 | |
| LY3415244 | | PD-L1, | Lilly, Zymeworks |
| | | TIM-3 | |
| GR1405 | | PD-L1 | Genrix Biopharma |
| LY3434172 | | PD-1, | Lilly, Zymeworks |
| | | PD-L1 | |
| CDX-527 | | CD27, | Celldex |
| | | PD-L1 | |
| FS222 | | 4-1BB, | f-star |
| | | PD-L1 | |
| LDP | | PD-L1 | Dragonboat Biopharma |
| ABL503 | | 4-1BB, | ABL Bio |
| | | PD-L1 | |
| HB0025 | | PD-L1, | Huabo Biopharm |
| | | VEGF | |
| MDX-1105 | BMS-936559, | PD-L1 | Medarex |
| | 12A4 | | |
| garivulimab | BGB-A333 | PD-L1 | BeiGene |
| GEN1046 | | 4-1BB, | BioNTech, Genmab |
| | | PD-L1 | |
| NM21-1480 | | 4-1BB, | Numab |
| | | PD-L1, | |
| | | Serum | |
| | | Albumin | |
| bintrafusp | M7824, | PD-L1, | Merck Serono, NCI |
| alfa | MSB0011359C | TGFβRII | |
| pacmilimab | CX-072 | PD-L1 | CytomX |
| A167 | KL-A167 | PD-L1 | Harbour Biomed Ltd., |
| | | | Sichuan Kelun Pharma |
| IBI318 | IBI-318 | PD-1, | Innovent, Lilly |
| | | PD-L1 | |
| KN046 | | CTLA4, | Alphamab |
| | | PD-L1 | |
| STI-3031 | IMC-001 | PD-L1 | Sorrento |
| SHR-1701 | | PD-L1 | Jiangsu Hengrui |
| LP002 | | PD-L1 | Taizhou HoudeAoke Bio |
| STI-1014 | ZKAB001 | PD-L1 | Lee's Pharm, Sorrento |
| envafolimab | KN035 | PD-L1 | Alphamab |
| adebrelimab | SHR-1316 | PD-L1 | Jiangsu Hengrui, |
| | | | Shanghai Hengrui |
| CS1001 | | PD-L1 | CStone Pharma |
| TQB2450 | CBT-502 | PD-L1 | CBT Pharma, Chia Tai |
| | | | Tianqing Pharma |

In some embodiments, the CTLA-4 inhibitor is selected from the group of inhibitors shown in Table 11.

TABLE 11
Exemplary CTLA4 Inhibitor Antibodies

| Name | Internal Name | Antigen | Company |
|---|---|---|---|
| ipilimumab | Yervoy, MDX-010, | CTLA4 | Medarex |
| | MDX101, 10D1, | | |
| | BMS-734016 | | |
| ATOR-1015 | ADC-1015 | CTLA4, OX40 | Alligator |
| vudalimab | XmAb20717 | CTLA4, PD-1 | Xencor |
| SI-B003 | | CTLA4, PD-1 | Sichuan Baili |
| | | | Pharma, |
| | | | Systimmune |
| MGD019 | | CTLA4, PD-1 | MacroGenics |
| MEDI5752 | | CTLA4, PD-1 | Medimmune |
| ADU-1604 | | CTLA4 | Aduro |
| BCD-145 | Q3W | CTLA4 | Biocad |
| CS1002 | | CTLA4 | CStone Pharma |
| REGN4659 | | CTLA4 | Regeneron |
| pavunalimab | XmAb22841 | CTLA4, LAG-3 | Xencor |
| AGEN1181 | | CTLA4 | Agenus |
| QL1706 | PSB205 | CTLA4, PD-1 | Sound Biologics |
| ADG126 | | CTLA4 | Adagene |
| KN044 | | CTLA4 | Changchun |
| | | | Intelli-Crown |
| ONC-392 | | CTLA4 | OncoImmune, |
| | | | Pfizer |
| BMS-986218 | | CTLA4 | BMS |
| BMS-986249 | | CTLA4 | BMS |
| BT-001 | TG6030 | CTLA4 | BioInvent |
| quavonlimab | MK-1308 | CTLA4 | Merck (MSD) |
| zalifrelimab | AGEN1884 | CTLA4 | Agenus, Ludwig |
| | | | Inst., Sloan- |
| | | | Kettering |
| AK104 | | CTLA4, PD-1 | Akeso |
| IBI310 | IBI-310 | CTLA4 | Innovent |
| KN046 | | CTLA4, PD-L1 | Alphamab |
| tremelimumab | ticilimumab, | CTLA4 | Amgen, |
| | CP-675206, | | Medimmune, |
| | clone 11.2.1 | | Pfizer |

In some embodiments, the immune checkpoint inhibitor is a small molecule drug. Small molecule checkpoint inhibitors are described, e.g., in WO2015/034820A1, WO2015/160641A2, WO2018/009505 A1, WO2017/066227 A1, WO2018/044963 A1, WO2018/026971 A1, WO2018/045142 A1, WO2018/005374 A1, WO2017/202275 A1, WO2017/202273 A1, WO2017/202276 A1, WO2018/006795 A1, WO2016/142852 A1, WO2016/142894 A1, WO2015/033301 A1, WO2015/033299 A1, WO2016/142886 A2, WO2016/142833 A1, WO2018/051255 A1, WO2018/051254 A1, WO2017/205464 A1, US2017/0107216 A1, WO2017/070089A1, WO2017/106634A1, US2017/0174679 A1, US2018/0057486 A1, WO2018/013789 A1, US2017/0362253 A1, WO2017/192961 A1, WO2017/118762 A1, US2014/199334 A1, WO2015/036927 A1, US2014/0294898 A1, US2016/0340391 A1, WO2016/039749 A1, WO2017/176608 A1, WO2016/077518 A1, WO2016/100608 A1, US2017/0252432 A1, WO2016/126646 A1, WO2015/044900 A1, US2015/0125491 A1, WO2015/033303 A1, WO2016/142835 A1, WO2019/008154 A1, WO2019/008152 A1, and WO2019023575A1.

In some embodiments, the PD-1 inhibitor is 2-[[4-amino-1-[5-(1-amino-2-hydroxypropyl)-1,3,4-oxadiazol-2-yl]-4-oxobutyl]carbamoylamino]-3-hydroxypropanoic acid (CA-170).

In some embodiments, the immune checkpoint inhibitor is (S)-1-(3-Bromo-4-((2-bromo-[1,1′-biphenyl]-3-yl)methoxy)benzyl)piperidine-2-carboxylic Acid.

In some embodiments, the immune checkpoint inhibitor is a peptide. See, e.g., Sasikumar et al., “Peptide and Peptide-Inspired Checkpoint Inhibitors: Protein Fragments to Cancer Immunotherapy,” Medicine in Drug Discovery 8:100073 (2020).

### VII. Variants

In some embodiments, the fusion protein(s) or components thereof described herein, or the NK cell genotypes described herein, are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M.O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### VIII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “buffer solution” refers to an aqueous solution consisting of a mixture of a weak acid and its conjugate base, or vice versa.

As used herein, the term “cell culture medium” refers to a mixture for growth and proliferation of cells in vitro, which contains essential elements for growth and proliferation of cells such as sugars, amino acids, various nutrients, inorganic substances, etc.

A buffer solution, as used herein, is not a cell culture medium.

As used herein, the term “bioreactor” refers to a culture apparatus capable of continuously controlling a series of conditions that affect cell culture, such as dissolved oxygen concentration, dissolved carbon dioxide concentration, pH, and temperature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule(s) or polynucleotide(s) of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “engineered cells,” “transformants,” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a fusion protein, as described herein.

The section headings used herein are for organizational purposes only and are not to be construed as limiting the subject matter described.

### IX. Examples

The following examples are included for illustrative purposes only and are not intended to limit the scope of the invention.

### Example 1: Off-the-Shelf NK Cell Therapy Platform

One example of a method by which NK cells were expanded and stimulated is shown in FIG. 1. A single unit of FDA-licensed, frozen cord blood that has a high affinity variant of the receptor CD16 (the 158 V/V variant, see, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).) and the KIR-B genotype (KIR B allele of the KIR receptor family, see, e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5:e15115 (2010)) was selected as the source of NK cells.

The cord blood unit was thawed and the freezing medium was removed via centrifugation. The cell preparation was then depleted of T cells using the QuadroMACS Cell Selection System (Miltenyi) and CD3 (T cell) MicroBeads. A population of 6×10⁸ total nucleated cells (TNC) were labelled with the MicroBeads and separated using the QuadroMACS device and buffer. Following depletion of T cells, the remaining cells, which were predominantly monocytes and NK cells, were washed and collected in antibiotic-free medium (CellgroSCGM). The cell preparation was then evaluated for total nucleated cell count, viability, and % CD3+ cells. As shown in FIG. 1, the cord blood NK cells were CD3 depleted.

The CD3− cell preparation was inoculated into a gas permeable cell expansion bag containing growth medium. The cells were co-cultured with replication incompetent engineered HuT-78 (eHUT-78) feeder cells to enhance expansion for master cell bank (MCB) production. The CellgroSCGM growth media was initially supplemented with nti-CD3 antibody (OKT3), human plasma, glutamine, and IL-2.

As shown in FIG. 1, the NK cells are optionally engineered, e.g., to introduce CARs into the NK cells, e.g., with a lentiviral vector, during one of the co-culturing steps.

The cells were incubated as a static culture for 12-16 days at 37° C. in a 5% CO₂ balanced air environment, with additional exchanges of media occurring every 2 to 4 days. After the culture expanded more than 100-fold, the cultured cells were harvested and then suspended in freezing medium and filled into cryobags. In this example, 80 bags or vials at 10⁸ cells per bag or vial were produced during the co-culture. The cryobags were frozen using a controlled rate freezer and stored in vapor phase liquid nitrogen (LN₂) tanks below −150° C. These cryopreserved NK cells derived from the FDA-licensed cord blood unit served as the master cell bank (MCB).

To produce the drug product, a bag of frozen cells from the MCB was thawed and the freezing medium was removed. The thawed cells were inoculated into a disposable culture bag and co-cultured with feeder cells, e.g., eHUT78 feeder cells to produce the drug product. In this example, the cells are cultured in a 50 L bioreactor to produce thousands of lots of the drug product per unit of cord blood (e.g., 4,000-8,000 cryovials at 10⁹ cells/vial), which are mixed with a cryopreservation composition and frozen in a plurality of storage vessels such as cryovials. The drug product is an off-the-shelf infusion ready product that can be used for direct infusion. Each lot of the drug product can be used to infuse hundreds to thousands of patients (e.g., 100-1,000 patients, e.g. with a target dose of 4×10⁹ cells).

### Example 2: Feeder Cell Expansion

As one example, suitable feeder cells, e.g., eHut-78 cells, were thawed from a frozen stock and expanded and cultured in a 125 mL flask in growth medium comprising RPMI1640 (Life Technologies) 89% v/v, inactivated fetal bovine serum (FBS) (Life Technologies) (10% v/v), and glutamine (hyclone) (2 mM) at or at about 37° C. and at or at about 3-7% CO₂ for or for about 18-24 days. The cells were split every 2-3 days into 125 mL-2L flasks. The cells were harvested by centrifugation and gamma irradiated. The harvested and irradiated cells were mixed with a cryopreservation medium (Cryostor CS10) in 2 mL cryovials and frozen in a controlled rate freezer, with a decrease in temperature of about 15° C. every 5 minutes to a final temperature of or of about −90° C., after which they were transferred to a liquid nitrogen tank or freezer to a final temperature of or of about −150° C.

After freezing, cell viability was greater than or equal to 70% of the original number of cells (here, at least 1.0×10⁸ viable cells/mL), and 85% or more of the cells expressed mTNF-α, 85% or more of the cells expressed mbIL-21+, and 85% or more of the cells expressed 4-1BBL.

### Example 3: NK Cell Expansion and Stimulation

As one example, suitable NK cells can be prepared as follows using HuT-78 cells transduced to express 4-1BBL, membrane bound IL-21 and mutant TNFalpha (“eHut-78P cells”) as feeder cells. The feeder cells are suspended in 1% (v/v) CellGro medium and are irradiated with 20,000 cGy in a gamma-ray irradiator. Seed cells (e.g., CD3-depleted PBMC or CD3− depleted cord blood cells) are grown on the feeder cells in CellGro medium containing human plasma, glutamine, IL-2, and OKT-3 in static culture at 37° C. The cells are split every 2-4 days. The total culture time was 19 days. The NK cells are harvested by centrifugation and cryopreserved. Thawed NK are administered to patients in infusion medium consisting of: Phosphate Buffered Saline (PBS 1×, FujiFilm Irvine) (50% v/v), albumin (human) (20% v/v of OctaPharma albumin solution containing: 200 g/L protein, of which ≥96% is human albumin, 130-160 mmol sodium; ≤2 mmol potassium, 0.064-0.096 mmol/g protein N-acetyl-DL-tryptophan, 0.064-0.096 mmol/g protein, caprylic acid, ad. 1000 ml water), Dextran 40 in Dextrose (25% v/v of Hospira Dextran 40 in Dextrose Injection, USP containing: 10 g/100 mL Dextran 40 and 5 g/100 mL dextrose hydrous in water) and dimethyl sulfoxide (DMSO) (5% v/v of Avantor DMSL solution with a density of 1.101 g/cm³ at 20° C.).

In some case, the seed cells are CD3-depleted cord blood cells. A cell fraction can be depleted of CD3 cells by immunomagnetic selection, for example, using a CliniMACS T cell depletion set ((LS Depletion set (162-01) Miltenyi Biotec).

Preferably, the cord blood seed cells are selected to express CD16 having the V/V polymorphism at F158 (Fc gamma RIIIa-158 V/V genotype) (Musolino et al. 2008 J Clin Oncol 26:1789). Preferably, the cord blood seed cells are KIR-B haplotype.

### Example 4: Cord Blood as an NK Cell Source

NK cells make up five to 15% of peripheral blood lymphocytes. Traditionally, peripheral blood has been used as the source for NK cells for therapeutic use. However, as shown herein, NK cells derived from cord blood have a nearly ten-fold greater potential for expansion in the culture systems described herein than those derived from peripheral blood, without premature exhaustion or senescence of the cells. The expression of receptors of interest on the surface of NK cells, such as those involved in the activation of NK cells on engagement of tumor cells, was seen to be more consistent donor-to-donor for cord blood NKs than peripheral-blood NK cells. The use of the manufacturing process described herein consistently activated the NK cells in cord blood in a donor-independent manner, resulting in a highly scaled, active and consistent NK cell product.

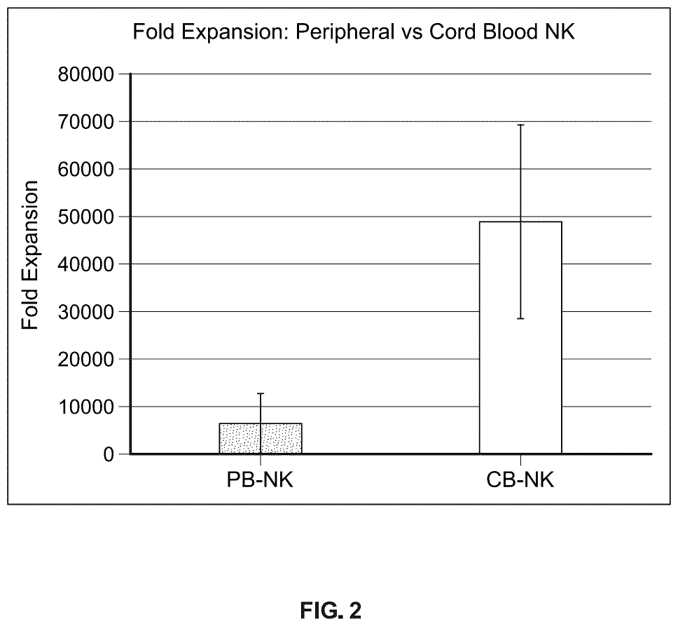

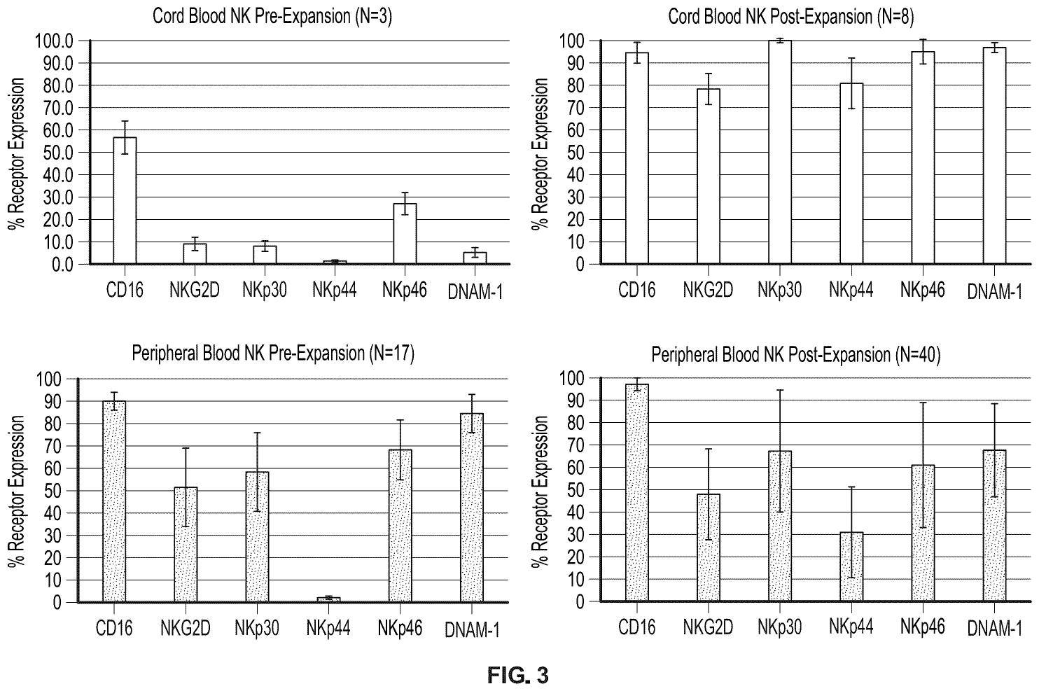

As shown in FIG. 2, cord blood-derived NK cells (CB-NK) have an approximately ten-fold greater ability to expand in culture than peripheral blood-derived NK cells (PB-NK) in preclinical studies. As shown in FIG. 3, expression of tumor-engaging NK activating immune receptors was higher and more consistent in cord blood-derived drug product compared to that generated from peripheral blood.

### Example 5: Expanded and Stimulated NK-Cell Phenotype

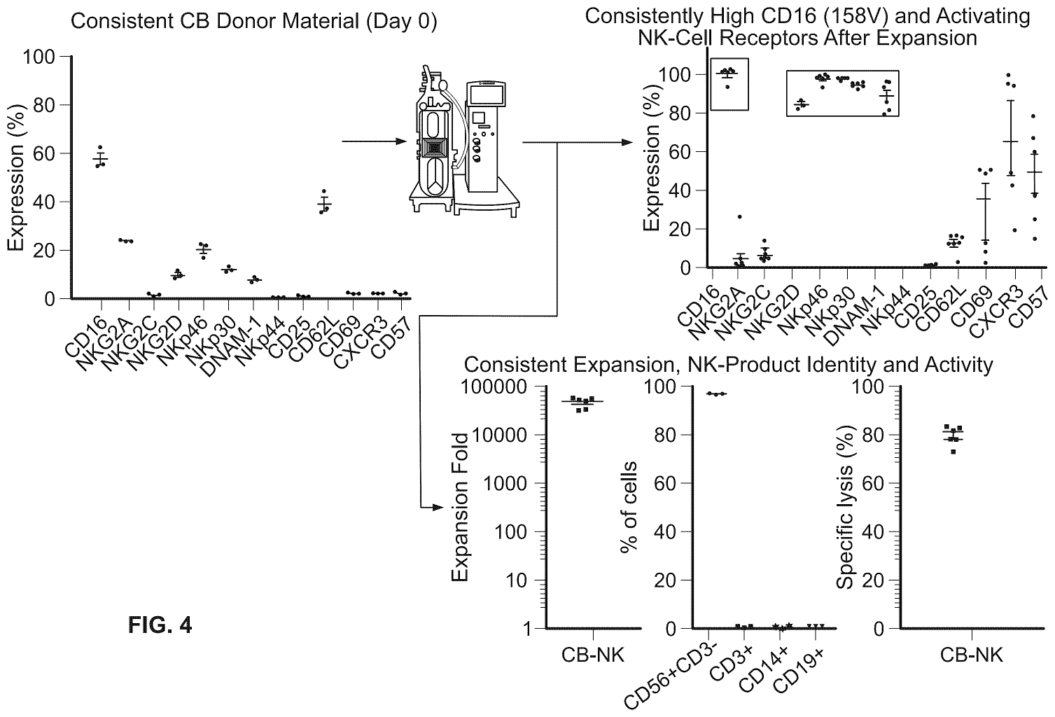

In one example, NK cells from a cord blood unit are expanded and stimulated with eHut-78 cells, according to the expansion and stimulation process described in Example 1. As shown in FIG. 4, the resulting expanded and stimulated population of NK cells have consistently high CD16 (158V) and activating NK-cell receptor expression.

### Example 6: AB-101

AB-101 is a universal, off-the-shelf, cryopreserved allogeneic cord blood derived NK cell therapy product comprising ex vivo expanded and activated effector cells designed to enhance ADCC anti-tumor responses in patients, e.g., patients treated with monoclonal antibodies or NK cell engagers. AB-101 is comprised of cord blood derived mononuclear cells (CBMCs) enriched for NK cells by depletion of T lymphocytes, and co-cultured with an engineered, replication incompetent T cell feeder line supplemented with IL-2 and anti-CD3 antibody (OKT3).

AB-101 is an allogeneic NK-cell product derived from FDA licensed cord blood, specifically designed to treat hematological and solid tumors in combination with therapeutic monoclonal antibodies (mAbs). The AB-101 manufacturing process leads to an NK cell product with the following attributes:
Consistent NK cell profile. High surface receptor expression of antibody engaging CD16 and tumor antigen-engaging/activating receptors such as NKG2D, NKp46, Nkp30 and NKp44.
KIR-B-haplotype. KIR-B haplotype has been associated with improved clinical outcomes in the haploidentical transplant setting and greater therapeutic potential in the allogeneic setting
CD16 F158V polymorphism. The higher-affinity CD16 F158V variant binding to mAb Fc-domain is seen to facilitate enhanced antibody dependent cellular cytotoxicity (ADCC).
Unmodified NK cells. No genetic enhancement or gene editing is required for, or is a part of, the AB-101 drug product.

The components and composition of AB-101 are listed in Table 12. AB-101 is comprised of NK cells (CD16⁺, CD56⁺) expressing the natural cytotoxicity receptors NKp30 and NKp46 indicative of mature NK cells. AB-101 contains negligible T cells, B cells and macrophages (≤0.2% CD3⁺, ≤1.0% CD19⁺, ≤1.0% CD14⁺). Residual eHuT-78P feeder cells used in the culturing of AB-101 are ≤0.2% of the drug product.

TABLE 12
Components and Compositions of AB-101

| Component | Solution | | | Quantity per Unit |
|---|---|---|---|---|
| Solution | Composition | Conc | Conc | (11 mL fill) |
| AB-101 drug | Approximately | 50% | 0.5 mL/mL | 5.5 mL |
| substance | 1.1 × 10⁹ viable | v/v | | (0.9 × 10⁹-1.3 × 10⁹ |
| (ex vivo- | cells | | | viable cells per |
| expanded | | | | vial in 5.27-6.23 |
| allogeneic | | | | mL of PBS) |
| natural killer | | | | |
| cells) | | | | |
| PBS | 100% Phosphate | | | |
| | Buffered Saline | | | |
| | (PBS) | | | |
| Albumin | 200 g/L albumin | 20% | 40 mg/mL | 2.2 mL |
| Solution | in water | v/v | albumin | (1.98-2.42 mL) |
| Dextran 40 | 100 g/L Dextran | 25% | 25 mg/mL | 2.75 mL |
| Solution | 40; and | v/v | Dextran 40; | (2.475-3.025 mL) |
| | 50 g/L glucose | | 12.5 mg/mL | |
| | in water | | glucose | |
| DMSO | 100% DMSO | 5% | 55 mg/mL | 0.55 mL |
| | (1,100 g/L) | v/v | | (0.495-0.605 mL) |

Initial stability studies indicate that AB-101 is stable for up to six months in the vapor phase of liquid nitrogen. Long-term stability studies to assess product stability beyond six months are ongoing, and the most current stability information will be captured on the certificate of analysis.

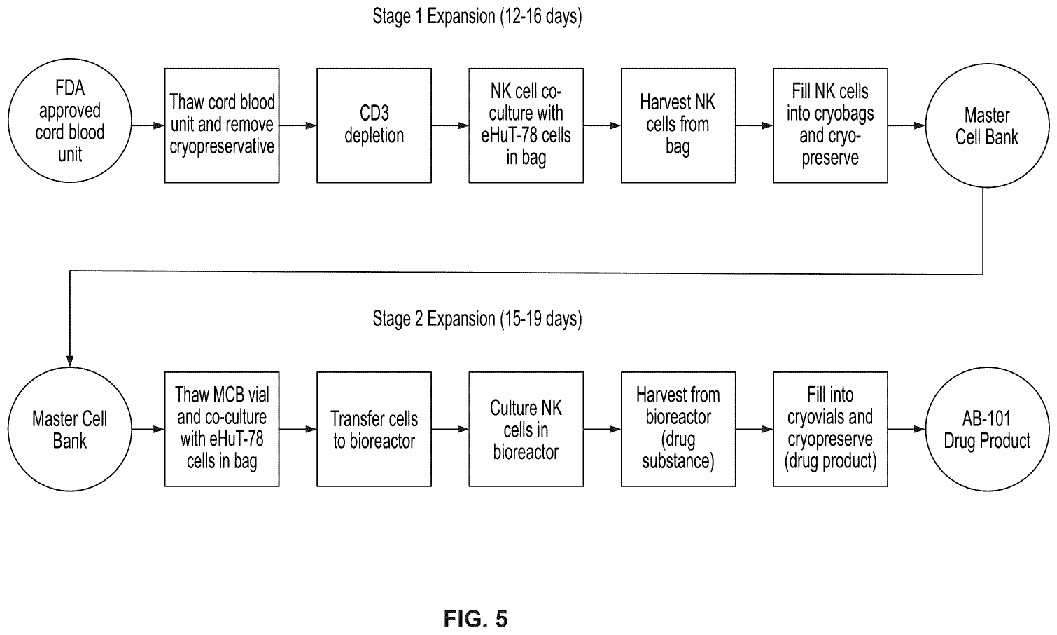

The manufacture of the AB-101 drug product is comprised of the following key steps (FIG. 5):
Thaw of the FDA licensed cord blood unit (Hemacord, BLA 125937).
Removal of cryo-preservation medium from the cord blood unit (CBU)
CD3 depletion using FDA cleared Vario MACS Cell Selection System (Miltenyi)
Expansion and co-culture in bags with an engineered feeder cell line (eHuT-78 cells)
Testing and cryopreservation of the AB-101 master cell bank (approximately 200 bags)
Thaw (single bag), expand and co-culture with engineered HuT-78 cells
Further expansion in bioreactor
Harvest and fill (1×10⁹ NK cells per vial)
Cryopreservation of the AB-101 drug product (approximately 150 vials)
Extensive characterization to determine consistency, purity, potency and safety.

As shown in Table 13, this manufacturing process reproducibly generates very large quantities of highly pure and active AB-101 drug product NK cells. Data points represent products generated from three independent cord blood units.

TABLE 13
AB-101 Product Characterization

| | | Acceptance | Engineering Batches | | | Clinical Batches | | | |
|---|---|---|---|---|---|---|---|---|---|
| Test Attribute | | Criterion | 1 | 2 | 3 | 1 | 2 | 3 | 4 |
| Cell Count | | 0.9-1.3 × 10⁹ | 1.3 × 10⁹ | 1.1 × 10⁹ | 1.0 × 10⁹ | 1.3 × 10⁹ | 1.2 × 10⁹ | 1.2 × 10⁹ | 1.0 × 10⁹ |
| (cells/vial) | | | | | | | | | |
| Cell Viability | | ≥70% | 96% | 95% | 94% | 93% | 94% | 94% | 94% |
| Endotoxin (EU/mL) | | ≤5 | ≤1 | ≤1 | ≤1 | ≤1 | ≤1 | ≤1 | ≤1 |
| Identity | CD3−, | ≥85% | 99.16% | 99.79% | 99.43% | 99.53% | 98.40% | 97.87% | 98.54% |
| | CD56+ % | | | | | | | | |
| | CD56+, | ≥70% | 94.42% | 94.20% | 99.04% | 93.24% | 91.72% | 95.22% | 90.21% |
| | CD16+ % | | | | | | | | |
| Purity | CD3+ % | (CD3+) ≤0.20% | ≤0.00% | 0.00% | 0.00% | 0.06% | 0.00% | 0.00% | 0.02% |
| | CD14+ % | (CD14+) ≤1.00% | ≤0.02% | 0.00% | 0.00% | 0.02% | 0.03% | 0.01% | 0.10% |
| | CD19+ % | (CD19+) ≤1.00% | ≤0.01% | 0.01% | 0.00% | 0.00% | 0.00% | 0.05% | 0.05% |

| Potency | ≥50% | 69.00% | 60.20% | 64.10% | 64.50% | 67.10% | 54.80% | 67.40% |
|---|---|---|---|---|---|---|---|---|
| | killing at 4 | | | | | | | |
| | hours | | | | | | | |

### Identity (CD3−, CD56+)

The frequency of CD3−, CD56+ cells are used to assess the identity of AB-101 Drug Product. A sample of AB-101 Drug Product is thawed and resuspended in a staining buffer. The resuspended sample is added to fluorochrome-labeled antibodies that bind to CD3+ and CD56+ surface antigens. Flow cytometry is used to determine percent populations of CD3−, CD56+as a measure of product identity.

### Identity (CD56+, CD16+)

The frequency of CD56+, CD16+ cells are used to assess the identity of AB-101 Drug Product. A sample of AB-101 Drug Product is thawed and resuspended in a staining buffer. The resuspended sample is added to fluorochrome-labeled antibodies that bind to CD56+ and CD16+ surface antigens. Flow cytometry is used to determine percent populations of CD56+, CD16+as a measure of product identity.

### Purity (CD3+)

Measurement of CD3+ expressing cells are used to assess the purity of AB-101 Drug Product. Flow cytometry method is used to determine the purity of the drug product for CD3+ expressing cells. The percent population of CD3+ cells is used as a measure of product purity.

### Purity (CD14+)

Measurement of CD14+ expressing cells are used to assess the purity of AB-101 Drug Product. Flow cytometry method is used to determine the purity of the drug product for CD14+ expressing cells. The percent population of CD14+ cells is used as a measure of product purity.

### Purity (CD19+)

Measurement of CD19+ expressing cells are used to assess the purity of AB-101 Drug Product. Flow cytometry method is used to determine the purity of the drug product for CD19+ expressing cells. The percent population of CD19+ cells is used as a measure of product purity.

Purity: Residual eHuT-78P (Residual eHuT-78P Cells)

Residual eHuT-78P cells in AB-101 drug product are measured by flow cytometry (FACS). FACS is used detect residual eHuT-78 in AB-101 DP by quantifying the live CD3+4-1BBLhigh+eHuT-78P. The FACS gating strategy (See FIG. 1), which sequentially gates, singlet, 7-AAD and CD3+4-1BBL+, was used because eHuT-78 is derived from a HuT-78 cell line that expresses CD3 as cutaneous T lymphocyte. The HuT-78 cell line was transduced by 4-1BB ligand (4-1BBL), membrane tumor necrosis factor-a (mTNF-a) and membrane bound IL-21 (mbIL-21). An eHuT-78 single cell that highly expresses the three genes was selected, and research, master and working cell banks were successively established. Among the three genes, 4-1BBL was utilized for the FACS gating strategy because it showed the highest expression in AB-101 cell bank and final drug product.

### Potency (Cytotoxicity at 10:1 AB-101 DP Cells to K562 Cells)

Potency of AB-101 Drug Product is determined by evaluating capacity for cellular cytotoxicity against K562 tumor cells. Cytotoxicity of the drug product will be assessed by fluorometric assay. K562 tumor cells are stained with 30 μM calcein-AM (Molecular probe) for 1 hour at 37° C. A sample of the drug product and the labeled tumor cells are co-cultured in a 96-well plate in triplicate at 37° C. and 5% CO2 for 4 hours with light protection. RPM11640 medium containing 10% FBS or 2% triton-X100 was added to the targets to provide spontaneous and maximum release. RPMI1640 medium containing 10% FBS or 2% triton-X100 is added to each well to determine background fluorescence. The measurement of fluorescence is conducted at excitation of 485 nm and emission 535 nm with a florescent reader. The percent specific cytotoxicity is calculated by the following formula.

$$\% Specific cytotoxicity = 100 \times \frac{\% specific death - \% spontaneous death}{100 - \% spontaneous death}$$

### Potency (Cytotoxicity at 10:1 AB-101 DP Cells to Ramos Cells)

Potency of AB-101 Drug Product is also determined by evaluating the capacity for cellular cytotoxicity against Ramos tumor cells using the same method and calculation described above. The specification for this testing is being determined.

### Example 7: AB-101 Phenotypic Characterization

The purity as well as expression of antibody-engaging CD16 and activating, inhibitory and chemokine receptors of multiple batches of AB-101 were measured via flow cytometry.

AB-101 purity was measured using cell surface markers: AB-101 batches were seen to comprise >99% CD3-CD56+NK cells and <0.1% CD3+, CD14+ and CD19+ cells. CD16 expression of AB-101 was measured. 95.11±2.51% of AB-101 cells were CD16+ with mean and median MFI of CD16 15311±6186 and 13097±5592 respectively. NK cells are known to express various NK specific activating and inhibitory receptors. For the various AB-101 batches that were tested, >80% of cells expressed CD16, NKG2A, NKG2D, CD94, NKp30, 2B4, Tim-3, CD44, 40˜70% of cells expressed NKp44, NKp46, DNAM-1, approximately 30% of cells expressed CD161 and CD96, 15% of cells expressed CXCR3, and less than 5% of cells expressed other activating inhibitory receptors.

Two GMP batches of AB-101 were included in the study to assess the phenotypic characteristics of NK cells at three different stages of the manufacturing process: Cord blood cells post CD3+ cell depletion; master cell bank (MCB) as intermediate, and AB-101 final drug product (DP). The CD3 depleted cells, MCB and DP, each were measured for purity and NK cell receptors. Based on the results, it was seen that NK cells initially derived from CB showed immature NK phenotypes. The NK phenotype matured during the manufacturing process. At the MCB stage, more than 90% of cells already expressed the phenotypic characteristic seen in matured NK cells, and markers of other cell types were <0.1%. The expression level for most of the NK cell-specific receptors increased throughout the manufacturing process from CD3 depleted cells, to MCB and finally DP

List of Abbreviations: NK Natural killer; mAb Monoclonal antibody; TNF-α Tumor necrosis factor alpha; CXCR CXC chemokine receptors; DNAM-1 DNAX Accessory Molecule-1; CRACC CD2-like receptor-activating cytotoxic cell; ILT2 Ig-like transcript 2; Tim-3 T-cell immunoglobulin mucin-3; 7AAD 7-amino-actinomycin D; ULBP UL16-binding protein; MICA/B MHC class I chain-related protein A and B; RAE1 Ribonucleic Acid Export 1; H60 NKG2D interacts with two cell surface ligands related to class; I MHC molecules; MULTI mouse UL16-binding protein-like transcript 1; MHC Major histocompatibility complex; HLA Human Leukocyte Antigen

Phenotype and purity staining protocol: 1. Adjust NK cell concentration at 2.0×10⁶ cells/mL in cold FACS buffer. 2. Refer to the table below, make an antibody mixture. 3. Add and mix antibody mixture with 100Lp diluted cells in a 5 mL round bottom tube. 4. Stain the cells for 30 minutes under blocking light and 4° C. conditions. 5. After staining, add 2 mL of FACS and then centrifuge for 3-minutes under 2000 rpm and 4° C. conditions. 6. Discard supernatant and vortex the cell pellet. Then add 200 μL of FACS buffer. 7. Analyze cells on the flow cytometer (LSR Fortessa). 8. Analyze the expression level of each marker by using Flow-Jo software. 9. Gate phenotype as follow gating option. a. Gate singlet in FSC-A/FSC-H panel b. Gate live cell in 7-AAD/SSC-A panel c. Gate lymphocyte in FSC-A/SSC-A panel d. Gate NK cell(CD3− CD56+) in CD3/CD56 e. Draw quadrant according to isotype control and then analyze CD3/CD56, CD16/CD56, and CD14/CD19. f. Based on Fluorescence Minus One (FMO) in NK cells gating, each PE fluorescent expression of the markers (no. 1 and 3-30 in the table 1, % of expression) is counted. In case of CD16, mean ratio and median is counted.

A list of antibody combinations for NK cell phenotype staining is shown in Table 14.

TABLE 14
List of antibody combinations for NK cell phenotype staining

| | FITC | | PE-Cy7 | PerCP-Cy5.5 |
|---|---|---|---|---|
| | (Fluorescein | PE | (Phyco- | (Peridinin- |
| | isothio- | (phyco- | erythrin- | chlorophyll-protein |
| No. | cyanate) | erythrin) | Cyanine7) | Complex: CY5.5 |
| 1 | CD3 | CD16 | CD56 | 7-AAD |
| 2 | CD14 | CD19 | CD3 | |
| 3 | CD3 | NKG2A | CD56 | |
| 4 | | NKG2C | | |
| 5 | | NKG2D | | |
| 6 | | NKp30 | | |
| 7 | | NKp44 | | |
| 8 | | NKp46 | | |
| 9 | | NKp80 | | |
| 10 | | CXCR3 | | |
| 11 | | CXCR4 | | |
| 12 | | CXCR5 | | |
| 13 | | CXCR6 | | |
| 14 | | CD195 | | |
| 15 | | CD244 | | |
| 16 | | DNAM-1 | | |
| 17 | | CD44 | | |
| 18 | | CD57 | | |
| 19 | | CD62L | | |
| 20 | | CD69 | | |
| 21 | | CD94 | | |
| 22 | | CD96 | | |
| 23 | | CD161 | | |
| 24 | | CRACC | | |
| 25 | | ILT-2 | | |
| 26 | | OX40L | | |
| 27 | | Tim-3 | | |
| 28 | | mIgG1 | | |
| (FMO) | | | | |
| 29 | mIgG1 | mIgG1 | mIgG1 | |
| (Isotype) | | | | |

Purity of AB-101 (n−9)

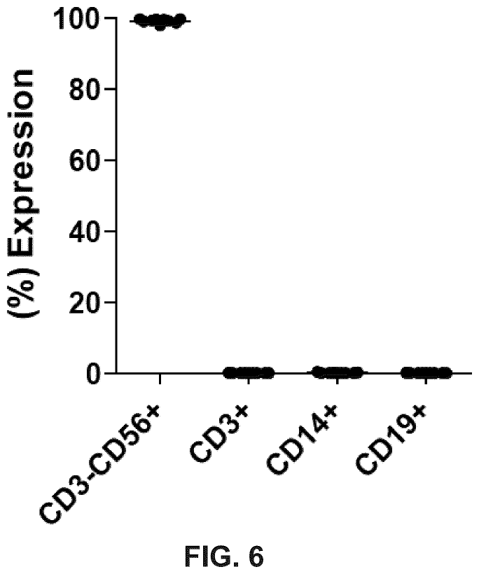

The purity of AB-101 is represented as CD3−CD56+ cells for NK cells, CD3+ cells for T-cells, CD14+ cells for monocytes and CD19+ cells for B-cells. Total 9 batches of AB-101 were measured for the purity. The results showed 99.27±0.59% (mean±SD) for CD3−CD56+ cells, 0.02±0.03% for CD3+ cells, 0.10±0.12% for CD14+ cells, and 0.02±0.04% for CD19+ cells (FIG. 6). Therefore, it was confirmed that AB-101 is composed of high-purity of NK cells, and the other types of cells as impurities were rarely present.

### Comparison of Purity of CD3 Depleted Cells, MCB, and DP Manufactured in GMP Conditions.

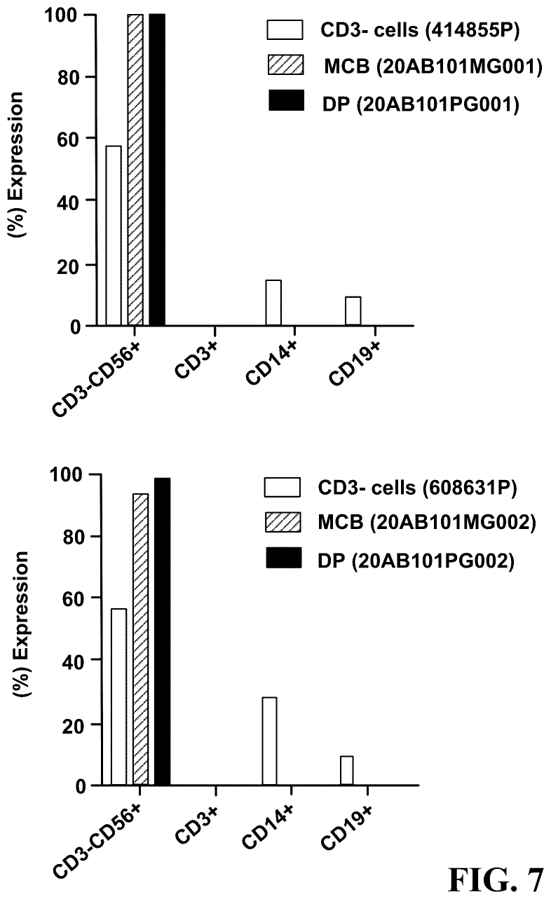

Two GMP batches of AB-101 were utilized to assess the purity of AB-101 starting material (CD3 depleted cells), intermediate (master cell bank, MCB), and final drug product (DP). 50˜60% of cells in CD3 depleted cell fraction were NK cells, and these percentages increased to more than 90% in MCB and DP. CD14+ cells and CD19+ cells were representative of 20˜30% of CD3 depleted cell fraction, and these cell percentages decreased to less than 0.1% in MCB and DP indicative of purity of AB-101 MCB and AB-101 final drug products (FIG. 7, Table 15).

TABLE 15
Cell Purity

| | GMP batch #1 | | | GMP batch #2 | | |
|---|---|---|---|---|---|---|
| | CD3- | MCB | DP | CD3- | MCB | DP |
| | cells | (20AB101 | (20AB101 | cells | (20AB101 | (20AB101 |
| Marker | (414855P) | MG001) | PG001) | (608631P) | MG002) | PG002) |
| CD3−CD56+ (%) | 58.0 | 99.43 | 99.80 | 56.70 | 93.14 | 97.98 |
| CD3+ (%) | 0.79 | 0.05 | 0.01 | 0.21 | 0.03 | 0.02 |
| CD14+ (%) | 15.01 | 0.02 | 0.01 | 28.00 | 0.03 | 0.02 |
| CD19+ (%) | 9.83 | 0.01 | 0.00 | 9.17 | 0.00 | 0.00 |

### Comparison of NK Cell Receptors of CD3 Depleted Cells, MCB, and DP Manufactured in GMP Conditions

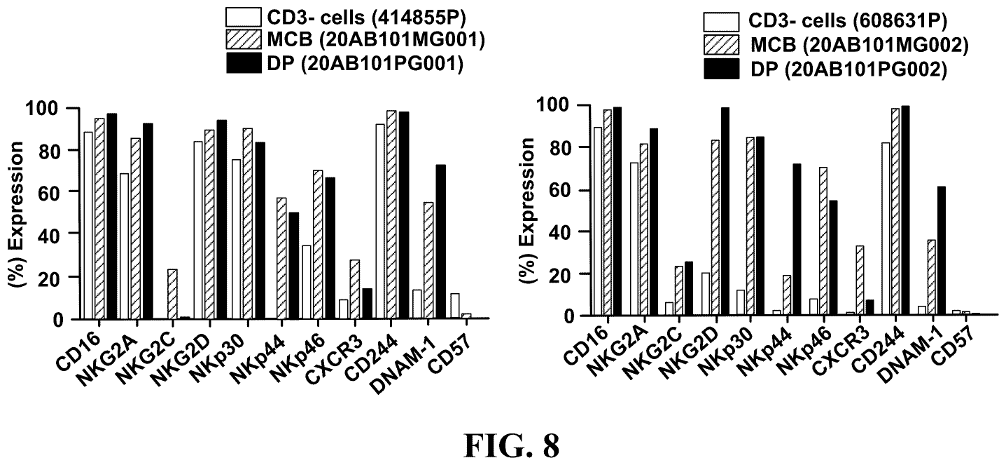

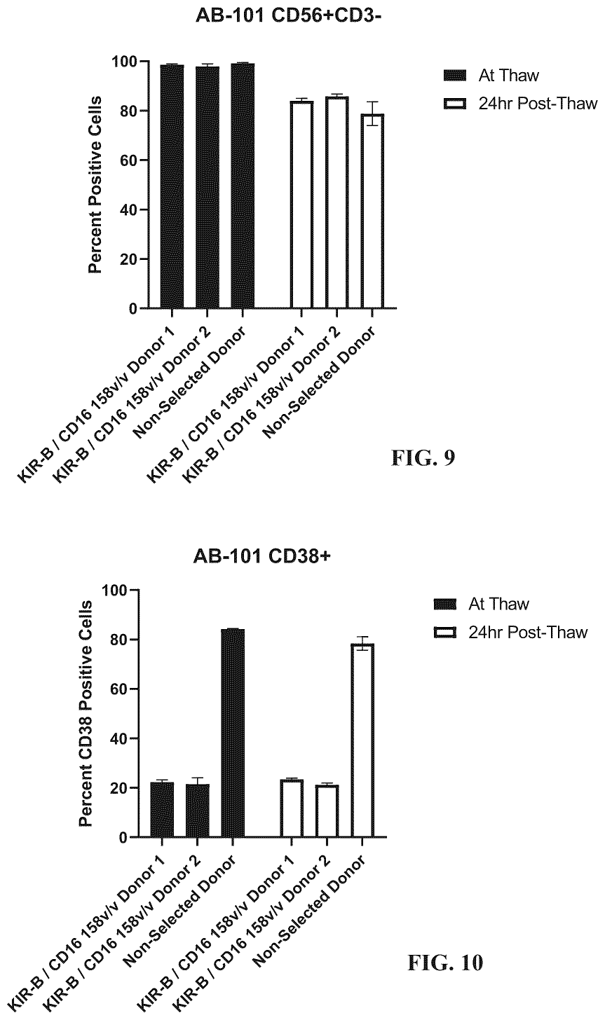

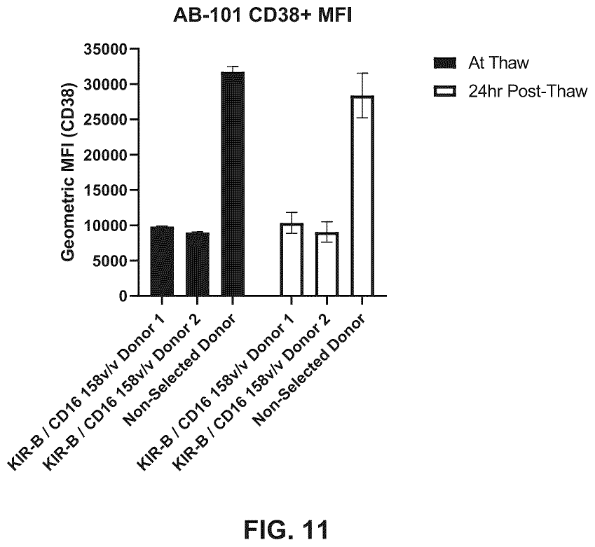

Two GMP batches of AB-101 were also utilized to assess the expression of various NK cell receptors on AB-101 starting material (CD3 depleted cells), intermediate (master cell bank, MCB), and final drug product (DP). It was observed that several NK cell and activating receptors such as CD16, NKG2D, NKG2C, NKp30, NKp44, NKp46 and DNAM-1 were expressed in higher levels by MCB, final drug product when compared to AB-101 starting material (CD3 depleted cells). The CD57 expression was lower in MCB and final drug product when compared to AB-101 starting material (CD3 depleted cells) (FIG. 8). Overall, data shows an increase in expression of NK cell activating receptors in MCB and DP indicative of AB-101 being effective against tumors.

TABLE 16
Cell Receptor Expression

| | GMP batch #1 | | | GMP batch #2 | | |
|---|---|---|---|---|---|---|
| | CD3- | MCB | DP | CD3- | MCB | DP |
| | cells | (20AB101 | (20AB101 | cells | (20AB101 | (20AB101 |
| Marker | (414855P) | MG001) | PG001) | (608631P) | MG002) | PG002) |
| Cd16 | 90.27 | 96.45 | 98.50 | 89.27 | 97.70 | 98.30 |
| NKG2A | 69.99 | 87.05 | 93.70 | 72.94 | 81.92 | 88.43 |
| NKG2C | 0.26 | 23.87 | 1.11 | 6.32 | 22.91 | 25.04 |
| NKG2D | 85.52 | 91.13 | 95.17 | 20.70 | 83.16 | 98.77 |
| NKp30 | 76.29 | 91.55 | 94.64 | 12.61 | 85.19 | 85.22 |
| NKp44 | 1.29 | 58.27 | 51.14 | 2.48 | 19.15 | 72.03 |
| NKp46 | 35.12 | 71.83 | 67.77 | 7.64 | 70.54 | 54.46 |
| CXCR3 | 9.10 | 28.39 | 14.40 | 1.79 | 33.13 | 7.01 |
| 2B4 | 93.66 | 99.75 | 99.20 | 82.63 | 98.29 | 99.46 |
| DNAM-1 | 13.94 | 55.64 | 73.07 | 5.12 | 36.24 | 61.13 |
| CD57 | 12.24 | 1.92 | 0.65 | 2.63 | 1.63 | 0.74 |

### Conclusion

The use of surface marker analysis supported the identity and purity and batch-to-batch consistency of the AB-101 product. Further, extensive assessment of NK-specific activating and inhibitory cell surface markers established the consistent profile of the AB-101 product post manufacturing expansion process. It is known that CB derived NK cells have immature phenotype such as high expression of NKG2A and low expression of NKG2C, CD62L, CD57, IL-2R, CD16, DNAM-1 comparing to peripheral blood (PB) derived NK cells, and it is also known that CB derived NK cells with the immature phenotypes exhibit low cytotoxicity against tumor cells. Data from this report shows that AB-101, an allogeneic cord blood (CB) derived NK cell product, expresses high levels of major activating receptors indicative of potential higher cytotoxicity against tumor cells.

### Example 8: AB-101 Pharmacokinetics and Biodistribution

The NOD scid gamma (NSG) mouse model was used to determine the biodistribution and pharmacokinetics (PK) of AB-101. Vehicle (PBS, Dextran, Albumin (human) DMSO) and AB-101 cells (0.5×10⁷ cells/mouse, 2×10⁷ cells/mouse) were administered intravenously (0.25 mL/mouse) for a total of 8 doses. Animals in vehicle and AB-101 groups were sacrificed at timepoints 4 hr, 1, 3, 7, 14 and 78 days (n=3 male mice, n=3 female mice per timepoint) post last dose infusion.

AB-101 was detected predominantly in highly perfused tissues (lungs, spleen, heart and liver) and at the site of injection starting at 4 hrs after administration, until 3 days after administration of final dose of AB-101 (day 53). At 7 days after administration of final dose (day 57) AB-101 was detected in lung (3 out of 6 samples), spleen (5 out of 6 samples) and injection site (5 out of 6 samples). At 14 days and 28 days after administration of final dose (day 64 and day 78 respectively), AB-101 was detected in two and one injection site samples, respectively. The sporadic incidence and low concentrations observed from the injection site samples at day 64 and day 78 would not be indicative of systemic persistence of the AB-101 test article.

The results from the biodistribution studies indicate that the distribution of AB-101 in vivo is consistent with the intravenous route of administration and that the cells lack long-term persistence potential with tissue clearance after 7 days post-administration and no evidence of permanent engraftment.

### Example 9: AB-101 Toxicology

Nonclinical toxicity of AB-101 was assessed in a GLP study of NSG mice. The study was designed to evaluate the acute and delayed toxicity profile of AB-101. Two dose levels of AB-101, 0.5×10⁷ and 2×10⁷ cells/animal, were tested in the study. The proposed test dose range was designed to deliver a greater exposure of the product than the planned highest equivalent human dose to be given in a first-in-human study (4×10⁹ cells per dose). Based on allometric scaling (Nair 2016), 0.5×10⁷ cells/mouse corresponded to 14×10⁹ cells/human, and 2×10⁷ cells/mouse corresponded to 56×10⁹ cells/human, assuming a patient weighing 70 kg. AB-101 was administered intravenously once weekly for 8 weeks via the tail vein. Acute toxicity of AB-101 was evaluated 3 days after the eighth dose (i.e., last dose). Delayed toxicity was evaluated at the end of the 28 days recovery period after the eighth dose. Viability, body weight, clinical observations and palpations were recorded for each animal during the in-life portion of the study. Gross necropsy and sample collection for hematology, clinical chemistry and histopathology analysis were performed at the time of euthanasia for all animals.

Each group contained 20 animals in total, with 10 of each gender, to evaluate findings in both sexes and for powered statistical analysis. A vehicle treated control group was included for comparison to the AB-101 treated groups. To minimize treatment bias, animals were assigned to dose groups based on computer-generated (weight-ordered) randomization procedures, with male and females randomized separately. The study adhered to GLP guidelines, including those for data reporting.

No mortality and no adverse clinical observations were recorded related to administration of AB-101 at any of the evaluated dose levels. All minor clinical observations that were noted are common findings in mice and were not considered related to AB-101 administration. Body and organ weight changes were comparable among dose groups and different days of post-treatment assessment (Day 53 for acute toxicity groups and Day 78 for delayed toxicity groups). There were no AB-101-related changes in hematology and clinical chemistry parameters or gross necropsy findings noted in animals at euthanasia in either the acute or delayed toxicity groups. All fluctuations among individual and mean clinical chemistry values, regardless of statistical significance, were considered sporadic, consistent with biologic and procedure-related variation, and/or negligible in magnitude, and therefore deemed not related to AB-101 administration. There were no AB-101-related microscopic findings. In conclusion, results from the GLP toxicity study indicate that AB-101 is well tolerated in NSG mice with repeated dosing of up to 2×10⁷ cells/dose/animal.

### Example 10: Cryopreservation of NK Cells

AB-101 cells were prepared by the process shown in FIG. 5. At the end of the culture period the cells were harvested through the use of a Sartorius kSep® 400 Single-Use Automated Centrifugation System at Relative Centrifugal Field (RCF): 800-1200 g with a flow rate at 60 to 120 mL/min, and washed two times with Phosphate Buffer Solution (PBS). After washing, the AB-101 cells were formulated with: (1) Albumin (human); (2) Dextran 40; (3) DMSO and (4) PBS to a target concentration of 1×10⁸ cells/mL (exemplary cryopreservation composition #1, Table 4). The formulated suspension was then filled at a target volume of 11 mL into 10 mL AT-Closed Vial®. Filled vials were inspected, labeled and cryopreserved in a controlled rate freezer at ≤−135° C.

Stability studies were carried out with time=0 as the initial release testing data. The stability storage freezer is a validated vapor phase LN₂ storage freezer which is set to maintain a temperature of ≤−135° C. For sterility timepoints, 10% of the batch size or 4 vials, whichever is greater, was tested. Test articles were thawed at 37° C. to mimic clinical thawing conditions.

As shown in Table 17, viability and activity of cryopreserved AB-101 was shown to be preserved through at least nine months.

TABLE 17
Long Term Viability and Activity of Cryopreserved AB-101

| | | | Cryopreserved (≤135° C.), Sample | | | | | |
|---|---|---|---|---|---|---|---|---|
| | | | times (months) | | | | | |
| | | Acceptance | 0 | 3 | 6 | 9 | 12 | 18 |
| Test Attribute | | Criterion | months | months | months | months | months | months |
| Cell Count | | 0.9-1.3 × 10⁹ | 1.3 × 10⁹ | 1.3 × 10⁹ | 1.4 × 10⁹ | 1.4 × 10⁹ | 1.3 × 10⁹ | 1.4 × 10⁹ |
| (cells/vial) | | | | | | | cells/vial | cells/vial |
| Cell Viability | | ≥70% | ≥96% | 93% | 94% | 93% | 90% | 87% |
| Endotoxin | | ≤5 | ≤1 | ≤1 | ≤1 | ≤1 | <1.0 | <1.0 |
| (EU/kg/hr) | | | | | | | | |
| Identity | CD3−, | ≥85% | 99.16% | 99.39% | 99.49% | 99.41% | 99.54% | 99.36% |
| | CD56+ % | | | | | | | |
| | CD56+, | ≥70% | 94.42% | 94.60% | 94.44% | 93.71% | 94.85% | 90.27% |
| | CD16+ % | | | | | | | |
| Purity | CD3+ % | ≤0.20% | 0.00% | 0.00% | 0.00% | 0.04% | 0.06% | 0.00% |
| | CD14+ % | ≤1.00% | 0.02% | 0.00% | 0.00% | 0.02% | 0.01% | 0.00% |
| | CD19+ % | ≤1.00% | 0.01% | 0.00% | 0.01% | 0.02% | 0.00% | 0.00% |

| | | | | | | | |
|---|---|---|---|---|---|---|---|
| Potency (killing | ≥50% | 69.00% | 66.90% | 67.40% | 61.80% | 67.1 | 68.3 |
| at 4 hours) | | | | | | | |

To understand the stability characteristics of AB-101 during handling Just prior to administration, a “bedside” short-term stability study was performed. Samples were thawed, transferred to 10 mL syringes, filtered, and the contents stored in Falcon tubes, and kept at that temperature for defined time periods as shown. The collected product was then tested. Short-Term Stability Data for two lots of AB-101 is shown in Table 18.

TABLE 18
Short Term Stability Data for AB-101

| | | Lot | 0 | 5 | 15 | 30 | 60 | 90 | 120 | |
|---|---|---|---|---|---|---|---|---|---|---|
| Average data of 4 vials | | release | min | min | min | min | min | min | min | Flush |
| PG001 | Cell count | 1.18 | 1.10 | 1.11 | 1.11 | 1.10 | 1.12 | 1.07 | 1.03 | 0.07 |
| | (0.8 − 1.2 × | | | | | | | | | |
| | 10⁸ cells/mL) | | | | | | | | | |
| | Viability (%) | 93 | 94 | 94 | 94.75 | 94 | 93.5 | 93.5 | 93.5 | 93.25 |
| | CD3−56+ (%) | 99.53 | 99.53 | NT | NT | NT | 99.53 | NT | 97.58 | NT |
| | CD16−CD56 | 93.24 | 97.74 | NT | NT | NT | 97.74 | NT | 97.43 | NT |
| | (%) | | | | | | | | | |
| PG002 | Cell count | 1.09 | 1.13 | 1.08 | 1.14 | 1.14 | 1.08 | 1.11 | 1.05 | 0.08 |
| | (0.8 − 1.2 × | | | | | | | | | |
| | 10⁸ cells/mL) | | | | | | | | | |
| | Viability (%) | 94 | 93.75 | 94.25 | 94.75 | 95.25 | 94.25 | 94.5 | 94 | 92.75 |
| | CD3−56+ (%) | 98.40 | 99.30 | NT | NT | NT | 99.27 | NT | 99.53 | NT |
| | CD16+CD56 | 91.72 | 98.88 | NT | NT | NT | 99.55 | NT | 98.40 | NT |
| | (%) | | | | | | | | | |

### Example 11: AB-101 Demonstrates ADCC with Trastuzumab

Cytotoxicity of NK cells can be quantitatively measured at a range of NK cell (effector) to tumor cell (target) ratios. In one study, in which a HER2+ gastric carcinoma tumor cell line, NCI-N87, was grown in long-term culture for six days. Long-term cytotoxicity assays were performed utilizing phase contrast analysis of tumor cell confluence.

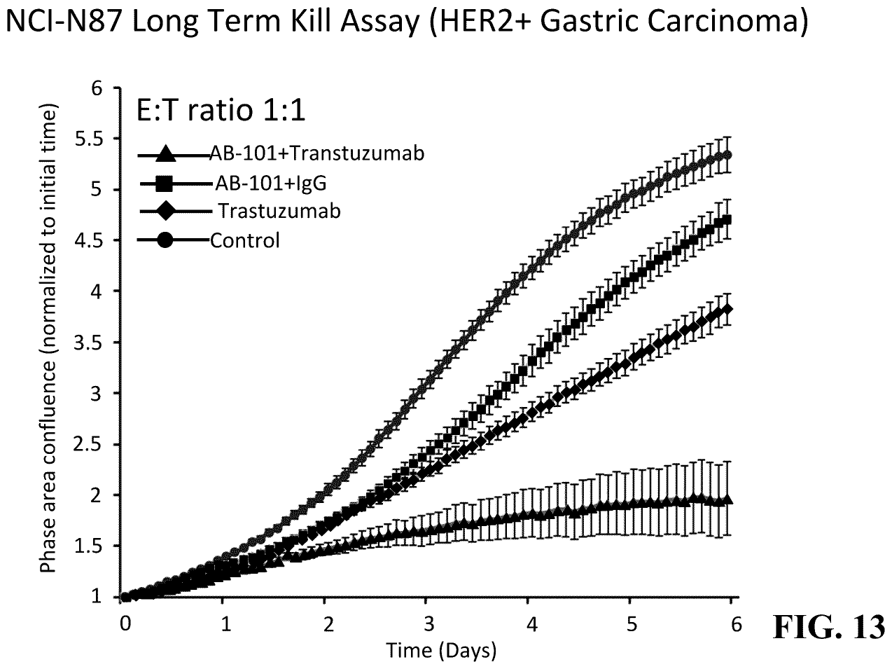

As shown in FIG. 13, trastuzumab was shown to inhibit the tumor cell growth and AB-101 was seen to kill the tumor line, but the combination of trastuzumab and AB-101 (E:T ration of 1:1) substantially increased the tumor killing cytotoxic activity.

### Example 12: AB-101+ Trastuzumab In Vitro Studies

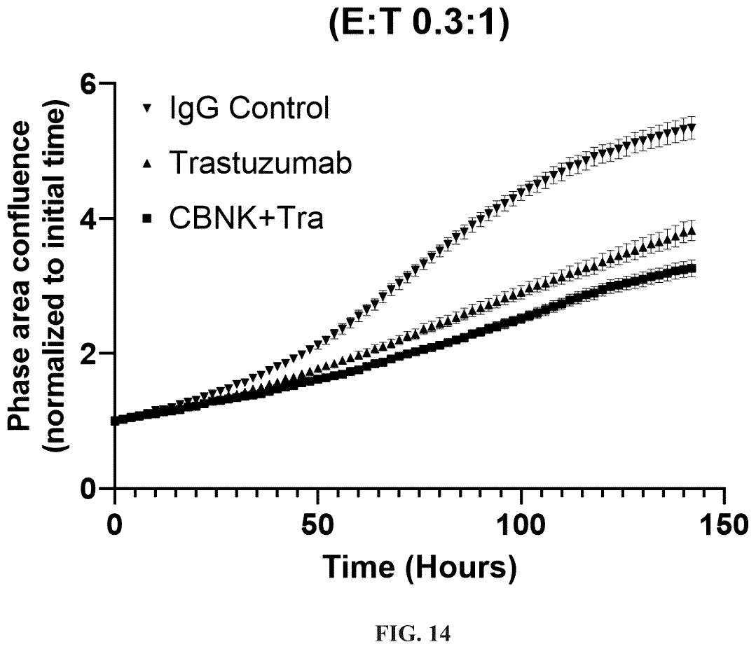

As shown in FIG. 14, the growth of a human HER+ gastric carcinoma cell line, NCI-N87, was monitored by measuring the cell confluence in long term cultures. Trastuzumab was seen to inhibit growth of the culture, whereas trastuzumab combined with the NK product AB-101 (E:T ratio of 0.3:1), resulted in further depletion of cell confluence via ADCC cell killing.

### Example 13: AB-101+ Trastuzumab In Vivo Studies

In vivo efficacy of AB-101+ trastuzumab has been evaluated in NOG mouse xenograft models bearing HER2+ tumors. The HER2+ xenograft models include intraperitoneal SKOV-3, HCC1954, and NCI-N87.

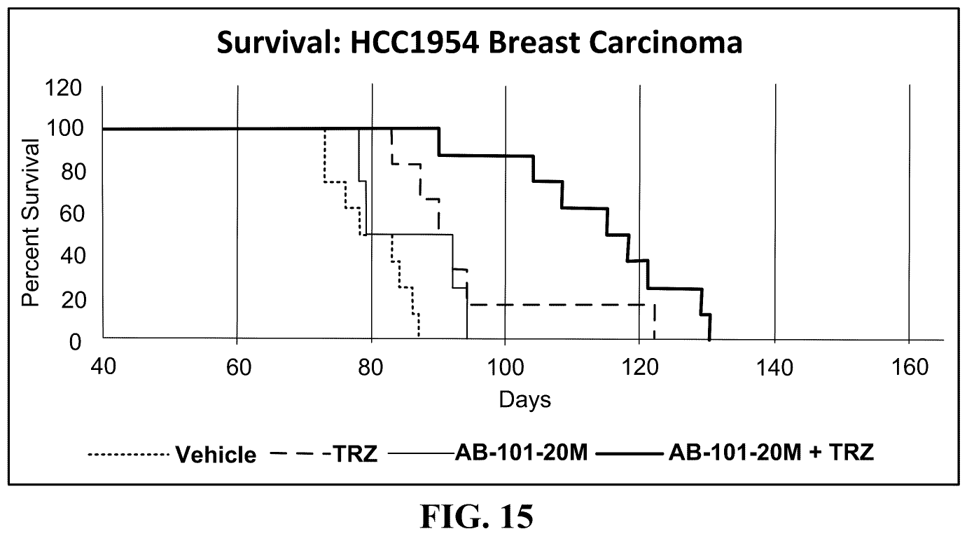

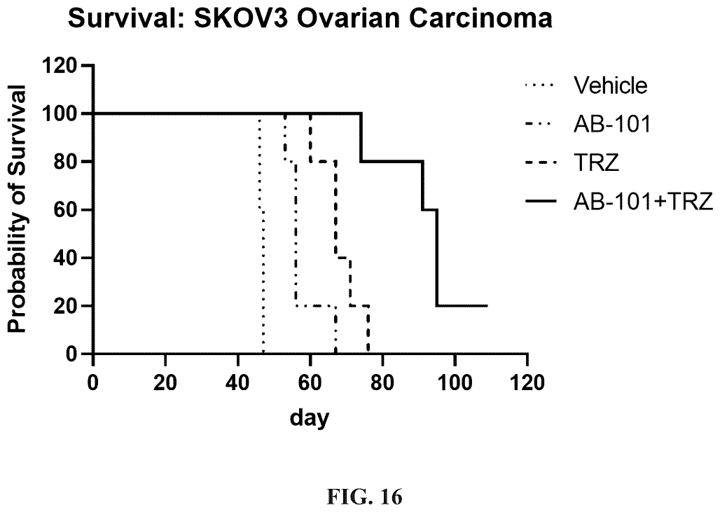

AB-101 in combination with trastuzumab killed tumor cells in a mouse xenograft model of breast cancer using the HCC1954 cell line, which has been characterized as trastuzumab resistant (FIG. 15), and in the SKOV3 Ovarian Carcinoma model (FIG. 16). HCC1954-luc tumor cells were grown in cell culture, harvested, and concentrated to 5×10⁶ cells/mL with PBS (phosphate buffered saline). Mice were injected intraperitoneally (IP) with 1×10⁶ cells/mouse. Three days after HCC1954-luc inoculation, mice were randomized to one of four groups (Table 19) according to bioluminescence of Day 0 (average bioluminescence signal was 2.49E+08 photons/s). AB101, AB201, TRZ and IL-2 were administered intraperitoneally.

TABLE 19

| | | | Volume | No. of |
|---|---|---|---|---|
| Group | Dose | Route | (μL) | Animals |
| 1 | Freezing medium + | i.p + i.p | 200 + 200 | 8 |
| | IgG 5 mg/kg | | | |
| 2 | TRZ 5 mg/kg | i.p | 200 | 8 |
| 3 | AB101 2 × 10⁷ cells + IL-2 | i.p + i.p | 200 + 100 | 8 |
| 4 | AB101 + TRZ + IL-2 | i.p + i.p + | 200 + | 8 |
| | | i.p | 200 + 100 | |

AB-101+ trastuzumab increased median survival time by 38.5 days. (FIG. 15).

In a separate experiment, NSG mice received 1×10⁶ SKOV3-Luc tumor cells (IP) on day 0 and a single injection of AB-201 (IP) on day 1IBioluminescence (BLI) measurements of SKOV3-Luc. AB-101+ trastuzumab increased median survival time. (FIG. 16).

### Example 14: Surface Protein Expression of AB-101

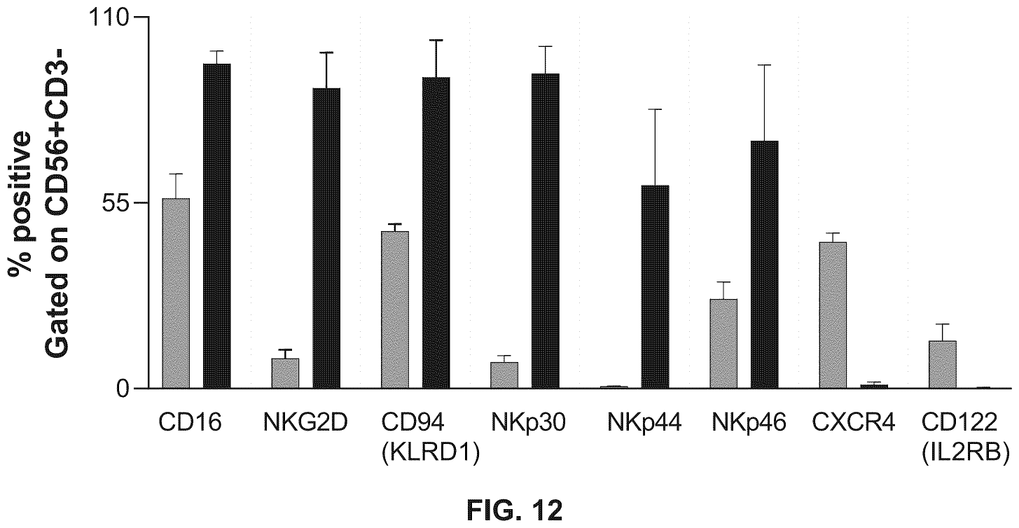

NK cells were expanded, as described in Example 6. Surface protein expression of the starting NK cell source (cord blood gated on CD56+/CD3− expression, n=3) was compared to the resulting expanded NK cells (n=16). As shown in FIG. 12, CD16 expression was high in the resulting cells, increased relative to the starting cells. Expression of NKG2D, CD94, NKp30, NKp44, and NKp46 was also increased, whereas expression of CXCR4 and CD122 was decreased.

SEQUENCES

| SEQ ID NO: and | |
|---|---|
| DESCRIPTION | SEQUENCE |
| SEQ ID NO: 1 | MEYASDASLDPEAPWPPAPRARACRVLPWALVAGLLLLLLLAAACAVELACPWAVSGA |
| Sequence of 4-1BBL | RASPGSAASPRLREGPELSPDDPAGLLDLRQGMFAQLVAQNVLLIDGPLSWYSDPGLA |
| that can be expressed | GVSLTGGLSYKEDTKELVVAKAGVYYVFFQLELRRVVAGEGSGSVSLALHLQPLRSAA |
| by feeder cells | GAAALALTVDLPPASSEARNSAFGFQGRLLHLSAGQRLGVHLHTEARARHAWQLTQGA |
| | TVLGLFRVTPEIPAGLPSPRSE |
| SEQ ID NO: 2 | MALPVTALLLPLALLLHAARPQDRHMIRMRQLIDIVDQLKNYVNDLVPEFLPAPEDVE |
| Sequence of a | TNCEWSAFSCFQKAQLKSANTGNNERIINVSIKKLKRKPPSTNAGRRQKHRLTCPSCD |
| membrane bound IL-21 | SYEKKPPKEFLERFKSLLQKMIHQHLSSRTHGSEDSAKPTTTPAPRPPTPAPTIASQP |
| (mbIL-21) that can | LSLRPEACRPAAGGAVHTRGLDFACDIYIWAPLAGTCGVLLLSLVITLY |
| be expressed by feeder | |
| cells | |
| SEQ ID NO: 3 | MSTESMIRDVELAEEALPKKTGGPQGSRRCLFLSLFSFLIVAGATTLFCLLHFGVIGP |
| Sequence of a mutated | QREEFPRDLSLISPLAQPVRSSSRTPSDKPVAHVVANPQAEGQLQWLNRRANALLANG |
| TNF alpha (mTNF-a) | VELRDNQLVVPSEGLYLIYSQVLFKGQGCPSTHVLLTHTISRIAVSYQTKVNLLSAIK |
| that can be expressed | SPCQRETPEGAEAKPWYEPIYLGGVFQLEKGDRLSAEINRPDYLDFAESGQVYFGIIA |
| by feeder cells | L |
| SEQ ID NO: 4 | MERVQPLEENVGNAARPRFERNKLLLVASVIQGLGLLLCFTYICLHFSALQVSHRYPR |
| Sequence of OX40L | IQSIKVQFTEYKKEKGFILTSQKEDEIMKVQNNSVIINCDGFYLISLKGYFSQEVNIS |
| that can be expressed | LHYQKDEEPLFQLKKVRSVNSLMVASLTYKDKVYLNVTTDNTSLDDFHVNGGELILIH |
| by feeder cells | QNPGEFCVL |
| SEQ ID NO: 5 | RSKRSRLLHSDYMNMTPRRPGPTRKHYQPYAPPRDFAAYRS |
| CD28 intracellular | |
| signaling domain | |
| SEQ ID NO: 6 | AGGAGTAAGAGGAGCAGGCTCCTGCACAGTGACTACATGAACATGACTCCCCGCCGCC |
| CD28 intracellular | CCGGGCCCACCCGCAAGCATTACCAGCCCTATGCCCCACCACGCGACTTCGCAGCCTA |
| signaling domain | TCGCTCC |
| SEQ ID NO: 7 | CGGAGCAAGAGGTCCCGCCTGCTGCACAGCGACTATATGAACATGACCCCACGGAGAC |
| Codon Optimized | CCGGCCCTACACGGAAACATTACCAGCCCTATGCTCCACCCCGGGACTTCGCAGCTTA |
| CD28 intracellular | CAGAAGT |
| signaling domain | |
| SEQ ID NO: 8 | ERVQPLEENVGNAARPRFERNK |
| OX40L intracellular | |
| signaling domain | |
| SEQ ID NO: 9 | GAAAGGGTCCAACCCCTGGAAGAGAATGTGGGAAATGCAGCCAGGCCAAGATTCGAGA |
| OX40L intracellular | GGAACAAG |
| signaling domain | |
| SEQ ID NO: 10 | GAAAGAGTGCAGCCCCTGGAAGAGAATGTCGGGAATGCCGCTCGCCCAAGATTTGAAA |
| Codon optimized | GGAACAAA |
| OX40L intracellular | |
| signaling domain | |
| SEQ ID NO: 11 | RVKFSRSADAPAYQQGQNQLYNELNLGRREEYDVLDKRRGRDPEMGGKPRRKNPQEGL |
| CD3ζ signaling domain | YNELQKDKMAEAYSEIGMKGERRRGKGHDGLYQGLSTATKDTYDALHMQALPPR |
| SEQ ID NO: 12 | AGAGTGAAGTTCAGCAGGAGCGCAGACGCCCCCGCGTACCAGCAGGGCCAGAACCAGC |
| CD3ζ signaling domain | TCTATAACGAGCTCAATCTAGGACGAAGAGAGGAGTACGATGTTTTGGACAAGAGACG |
| | TGGCCGGGACCCTGAGATGGGGGGAAAGCCGAGAAGGAAGAACCCTCAGGAAGGCCTG |
| | TACAATGAACTGCAGAAAGATAAGATGGCGGAGGCCTACAGTGAGATTGGGATGAAAG |
| | GCGAGCGCCGGAGGGGCAAGGGGCACGATGGCCTTTACCAGGGTCTCAGTACAGCCAC |
| | CAAGGACACCTACGACGCCCTTCACATGCAGGCCCTGCCCCCTCGC |
| SEQ ID NO: 13 | CGAGTGAAGTTCAGCAGGTCCGCCGACGCTCCTGCATACCAGCAGGGACAGAACCAGC |
| Codon optimized CD3ζ | TGTATAACGAGCTGAATCTGGGCCGGAGAGAGGAATACGACGTGCTGGACAAAAGGCG |
| signaling domain | GGGCCGGGACCCCGAAATGGGAGGGAAGCCACGACGGAAAAACCCCCAGGAGGGCCTG |
| | TACAATGAGCTGCAAAAGGACAAAATGGCCGAGGCTTATTCTGAAATCGGGATGAAGG |
| | GAGAGAGAAGGCGCGGAAAAGGCCACGATGGCCTGTACCAGGGGCTGAGCACCGCTAC |
| | AAAGGACACCTATGATGCACTGCACATGCAGGCCCTGCCCCCTCGG |
| SEQ ID NO: 14 | GSGEGRGSLLTCGDVEENPGP |
| T2A cleavage site | |
| SEQ ID NO: 15 | GGCTCAGGTGAGGGGCGCGGGAGCCTGCTGACTTGTGGGGATGTAGAGGAAAATCCTG |
| T2A cleavage site | GTCCT |
| SEQ ID NO: 16 | MRISKPHLRSISIQCYLCLLLNSHFLTEAGIHVFILGCFSAGLPKTEANWVNVISDLK |
| IL-15 | KIEDLIQSMHIDATLYTESDVHPSCKVTAMKCFLLELQVISLESGDASIHDTVENLII |
| | LANNSLSSNGNVTESGCKECEELEEKNIKEFLQSFVHIVQMFINTS- |
| SEQ ID NO: 17 | ATGAGAATCAGCAAACCACACCTCCGGAGCATATCAATCCAGTGTTACTTGTGCCTTC |
| IL-15 | TTTTGAACTCCCATTTCCTCACCGAGGCAGGCATTCATGTGTTCATATTGGGGTGCTT |
| | TAGTGCTGGGCTTCCGAAAACGGAAGCTAACTGGGTAAACGTCATCAGTGACCTTAAA |
| | AAAATTGAGGATCTTATCCAATCAATGCACATCGACGCGACTCTCTACACAGAATCTG |
| | ACGTACACCCGTCATGCAAAGTCACGGCAATGAAGTGTTTTCTTCTCGAGCTCCAAGT |
| | AATTTCCCTGGAGTCTGGCGATGCCTCCATCCACGATACGGTTGAAAATCTGATTATA |
| | TTGGCCAACAATAGCCTCAGTTCTAACGGTAACGTGACTGAAAGTGGCTGCAAAGAGT |
| | GCGAAGAGCTCGAAGAAAAGAATATCAAGGAGTTCCTCCAATCATTTGTTCACATTGT |
| | GCAAATGTTTATCAACACCTCTTGA |
| SEQ ID NO: 18 | ATGCGCATAAGTAAGCCTCATCTGCGGTCCATTTCTATACAATGTTATCTGTGCTTGC |
| IL-15 | TTTTGAACTCCCACTTTCTTACGGAAGCAGGCATTCATGTGTTCATTCTGGGTTGTTT |
| | TTCtGCCGGGCTGCCCAAAACCGAGGCCAACTGGGTCAACGTGATCAGCGACCTCAAG |
| | AAGATCGAGGATTTGATTCAAAGTATGCATATAGACGCCACACTCTATACTGAGTCCG |
| | ACGTTCACCCGAGTTGTAAAGTTACGGCTATGAAGTGCTTTTTGTTGGAACTCCAGGT |
| | GATTTCCCTTGAATCCGGCGATGCGAGCATCCACGATACGGTAGAGAATCTTATTATT |
| | CTGGCGAATAATTCTCTGTCTTCAAATGGGAATGTAACTGAGAGCGGTTGTAAAGAAT |
| | GCGAAGAACTTGAAGAAAAGAATATCAAGGAATTTCTTCAGAGTTTCGTGCATATTGT |
| | TCAAATGTTCATCAACACATCCTGA |
| SEQ ID NO: 19 | RSKRSRLLHSDYMNMTPRRPGPTRKHYQPYAPPRDFAAYRSERVQPLEENVGNAARPR |
| CD28/OX40L/CDζ | FERNKRVKESRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDALHMQALPP |
| | R |
| SEQ ID NO: 20 | RSKRSRLLHSDYMNMTPRRPGPTRKHYQPYAPPRDFAAYRSERVQPLEENVGNAARPR |
| CD28/OX40L/CDζ/ | FERNKRVKFSRSADAPAYQQGQNQLYNELNLGRREEYDVLDKRRGRDPEMGGKPRRKN |
| T2A/IL1-5 | PQEGLYNELQKDKMAEAYSEIGMKGERRRGKGHDGLYQGLSTATKDTYDALHMQALPP |
| | RGSGEGRGSLLTCGDVEENPGPMRISKPHLRSISIQCYLCLLLNSHELTEAGIHVFIL |
| | GCFSAGLPKTEANWVNVISDLKKIEDLIQSMHIDATLYTESDVHPSCKVTAMKCELLE |
| | LQVISLESGDASIHDTVENLIILANNSLSSNGNVTESGCKECEELEEKNIKEFLQSFV |
| | HIVQMFINTS- |
| SEQ ID NO: 21 | MKWVTFISLLELESSAYSRGVFRRDAHKSEVAHRFKDLGEENFKALVLIAFAQYLQQC |
| Human Albumin | PFEDHVKLVNEVTEFAKTCVADESAENCDKSLHTLFGDKLCTVATLRETYGEMADCCA |
| | KQEPERNECFLQHKDDNPNLPRLVRPEVDVMCTAFHDNEETFLKKYLYEIARRHPYFY |
| | APELLFFAKRYKAAFTECCQAADKAACLLPKLDELRDEGKASSAKQRLKCASLQKFGE |
| | RAFKAWAVARLSQRFPKAEFAEVSKLVTDLTKVHTECCHGDLLECADDRADLAKYICE |
| | NQDSISSKLKECCEKPLLEKSHCIAEVENDEMPADLPSLAADFVESKDVCKNYAEAKD |
| | VFLGMFLYEYARRHPDYSVVLLLRLAKTYETTLEKCCAAADPHECYAKVFDEFKPLVE |
| | EPQNLIKQNCELFEQLGEYKFQNALLVRYTKKVPQVSTPTLVEVSRNLGKVGSKCCKH |
| | PEAKRMPCAEDYLSVVLNQLCVLHEKTPVSDRVTKCCTESLVNRRPCESALEVDETYV |
| | PKEFNAETFTFHADICTLSEKERQIKKQTALVELVKHKPKATKEQLKAVMDDFAAFVE |
| | KCCKADDKETCFAEEGKKLVAASQAALGL |
| SEQ ID NO: 22 | ATGGCCACCGAGTACAAGCCCACGGTGCGCCTCGCCACCCGCGACGACGTCCCCCGGG |
| Puromycin Resistance | CCGTACGCACCCTCGCCGCCGCGTTCGCCGACTACCCCGCCACGCGCCACACCGTCGA |
| Gene | TCCGGACCGCCACATCGAGCGGGTCACCGAGCTGCAAGAACTCTTCCTCACGCGCGTC |
| | GGGCTCGACATCGGCAAGGTGTGGGTCGCGGACGACGGCGCCGCGGTGGCGGTCTGGA |
| | CCACGCCGGAGAGCGTCGAAGCGGGGGCGGTGTTCGCCGAGATCGGCCCGCGCATGGC |
| | CGAGTTGAGCGGTTCCCGGCTGGCCGCGCAGCAACAGATGGAAGGCCTCCTGGCGCCG |
| | CACCGGCCCAAGGAGCCCGCGTGGTTCCTGGCCACCGTCGGCGTCTCGCCCGACCACC |
| | AGGGCAAGGGTCTGGGCAGCGCCGTCGTGCTCCCCGGAGTGGAGGCGGCCGAGCGCGC |
| | CGGGGTGCCCGCCTTCCTGGAGACCTCCGCGCCCCGCAACCTCCCCTTCTACGAGCGG |
| | CTCGGCTTCACCGTCACCGCCGACGTCGAGGTGCCCGAAGGACCGCGCACCTGGTGCA |
| | TGACCCGCAAGCCCGGTGCCTGA |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a patient suffering from a HER2+ cancer, the method comprising administering a population of natural killer cells (NK cells) and an antibody targeted to human HER2, wherein the NK cells are allogenic to the patient, are KIR-B haplotype and homozygous for a CD16 158V polymorphism.

2. The method of claim 1, wherein the cancer is selected from the group consisting of breast cancer, gastric cancer, and ovarian cancer.

3. The method of claim 2, wherein the cancer is breast cancer.

4. The method of claim 2, wherein the cancer is gastric cancer.

5. The method of claim 2, wherein the cancer is ovarian cancer.

6. The method of claim 2, wherein the patient has relapsed after treatment with an anti-HER2 antibody.

7. The method of any one of the forgoing claims, wherein the patient has experienced disease progression after treatment with autologous stem cell transplant or chimeric antigen receptor T-cell therapy (CAR-T).

8. The method of any one of the forgoing claims, wherein the patient is administered 1×10⁸ to 1×10¹⁰ NK cells.

9. The method of any one of the forgoing claims, wherein the patient is administered 1×10⁹ to 8×10⁹ NK cells.

10. The method of any one of the forgoing claims, wherein the patient is administered 4×10⁸, 1×10⁹, 4×10⁹, or 8×10⁹ NK cells.

11. The method of any one of the forgoing claims, wherein the antibody is trastuzumab.

12. The method of any of the forgoing claims, wherein the patient is subjected to lymphodepleting chemotherapy prior to treatment.

13. The method of claim 13, wherein the lymphodepleting chemotherapy is non-myeloablative chemotherapy.

14. The method of claim 13 or claim 14, wherein the lymphodepleting chemotherapy comprises treatment with at least one of cyclophosphamide and fludarabine.

15. The method of claim 15, wherein the lymphodepleting chemotherapy comprises treatment with cyclophosphamide and fludarabine.

16. The method of any one of claims 15-16, wherein the cyclophosphamide is administered between 100 and 500 mg/m2/day.

17. The method of claim 16, wherein the cyclophosphamide is administered at 250 mg/m²/day.

18. The method of claim 16, wherein the cyclophosphamide is administered at 500 mg/m²/day.

19. The method of any one of claims 15-18, wherein the fludarabine is administered between 10 and 50 mg/m²/day.

20. The method of claim 19, wherein the fludarabine is administered 30 mg/m²/day.

21. The method of any of the forgoing claims further comprising administering IL-2.

22. The method of claim 21, wherein the patient is administered 1×10⁶ IU/m² of IL-2.

23. The method of claim 21, wherein the patient is administered 6 million IU of IL-2.

24. The method of any one of claims 21-23, wherein administration of IL-2 occurs within 1-4 hrs of administration of the NK cells.

25. The method of any of the forgoing claims wherein the administration of the NK cells and the antibody targeted to human HER2 occurs weekly.

26. The method of any of the forgoing claims wherein the NK cells and the antibody targeted to human HER2 are administered weekly for 4 to 8 weeks.

27. The method of any of the forgoing claims wherein the administration of the NK cells occurs weekly and the administration of the antibody targeted to human HER2 occurs every other week.

28. The method of any of the forgoing claims, wherein the NK cells are not genetically modified.

29. The method of any of the forgoing claims, wherein at least 70% of the NK cells are CD56+ and CD16+.

30. The method of any of the forgoing claims, wherein at least 85% of the NK cells are CD56+ and CD3−.

31. The method of any of the forgoing claims, wherein 1% or less of the NK cells are CD3+, 1% or less of the NK cells are CD19+ and 1% or less of the NK cells are CD14+.

32. The method of any of the forgoing claims wherein the each administration of NK cells is administration of 1×10⁹ to 5×10⁹ NK cells.

33. The method of any of the forgoing claims wherein the patient receives a dose of the HER2 targeting antibody before the first dose of NK cells.

34. The method of any of the forgoing claims, wherein the expanded natural killer cells are expanded umbilical cord blood natural killer cells.

35. The method of any of the forgoing claims, wherein the population of expanded natural killer cells comprises at least 60%, e.g., at least 70%, at least 80%, at least 90% at least 95%, at least 99%, or 100% CD16+ cells.

36. The method of any of the forgoing claims, wherein the population of expanded natural killer cells comprises at least 60%, e.g., at least 70%, at least 80%, at least 90% at least 95%, at least 99%, or 100% NKG2D+ cells.

37. The method of any of the forgoing claims, wherein the population of expanded natural killer cells comprises at least 60%, e.g., at least 70%, at least 80%, at least 90% at least 95%, at least 99%, or 100% NKp46+ cells.

38. The method of any of the forgoing claims, wherein the population of expanded natural killer cells comprises at least 60%, e.g., at least 70%, at least 80%, at least 90% at least 95%, at least 99%, or 100% NKp30+ cells.

39. The method of any of the forgoing claims, wherein the population of expanded natural killer cells comprises at least 60%, e.g., at least 70%, at least 80%, at least 90% at least 95%, at least 99%, or 100% DNAM-1+ cells.

40. The method of any of the forgoing claims, wherein the population of expanded natural killer cells comprises at least 60%, e.g., at least 70%, at least 80%, at least 90% at least 95%, at least 99%, or 100% NKp44+ cells.

41. The method of any of the forgoing claims, wherein the population of expanded natural killer cells comprises less than 20%, e.g., 10% or less, 5% or less, 1% or less, 0.5% or less, or 0% CD3+ cells.

42. The method of any of the forgoing claims, wherein the population of expanded natural killer cells comprises less than 20% or less, e.g., 10% or less, 5% or less, 1% or less, 0.5% or less, or 0% CD14+ cells.

43. The method of any of the forgoing claims, wherein the population of expanded natural killer cells comprises less than 20% or less, e.g., 10% or less, 5% or less, 1% or less, 0.5% or less, or 0% CD19+ cells.

44. The method of any of the forgoing claims, wherein the population of expanded natural killer cells comprises less than 20% or less, e.g., 10% or less, 5% or less, 1% or less, 0.5% or less, or 0% CD38+ cells.

45. The method of any of the forgoing claims, wherein the natural killer cells do not comprise a CD16 transgene.

46. The method of any of the forgoing claims, wherein the natural killer cells do not express an exogenous CD16 protein.

47. The method of any of the forgoing claims, wherein the expanded natural killer cells are not genetically engineered.

48. The method of any of the forgoing claims, wherein the expanded natural killer cells are derived from the same umbilical cord blood donor.

49. The method of any of the forgoing claims, wherein the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

50. The method of any of the forgoing claims, wherein the population of NK cells is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce expanded natural killer cells,
thereby producing the population of expanded natural killer cells.

51. The method of any of the forgoing claims, wherein the population of NK cells is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and
(d) expanding the master cell bank population of expanded natural killer cells by culturing with a second plurality of Hut78 cells engineered to express a membrane bound IL-21, a mutated TNFα, and a 4-1BBL gene to produce expanded natural killer cells;
thereby producing the population of expanded natural killer cells.

52. The method of claim 50 or claim 51, wherein the population of NK cells is produced by a method further comprising, after step (c),
(i) freezing the master cell bank population of expanded natural killer cells in a plurality of containers; and
(ii) thawing a container comprising an aliquot of the master cell bank population of expanded natural killer cells,
wherein expanding the master cell bank population of expanded natural killer cells in step (d) comprises expanding the aliquot of the master cell bank population of expanded natural killer cells.

53. The method of any one of claims 50 to 52, wherein the umbilical cord blood is from a donor with the KIR-B haplotype and homozygous for the CD16 158V polymorphism.

54. The method of any one of claims 50-53, wherein the population of NK cells is produced by a method comprising expanding the natural killer cells from umbilical cord blood at least 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold.

55. The method of any one of claims 50-54, wherein the population of expanded natural killer cells is not enriched or sorted after expansion.

56. The method of any one of claims 50-55, wherein the percentage of NK cells expressing CD16 in the population of expanded natural killer cells is the same or higher than the percentage of natural killer cells in the seed cells from umbilical cord blood.

57. The method of any one of claims 50-56, wherein the percentage of NK cells expressing NKG2D in the population of expanded natural killer cells is the same or higher than the percentage of natural killer cells in the seed cells from umbilical cord blood.

58. The method of any one of claims 50-57, wherein the percentage of NK cells expressing NKp30 in the population of expanded natural killer cells is the same or higher than the percentage of natural killer cells in the seed cells from umbilical cord blood.

59. The method of any one of claims 50-58, wherein the percentage of NK cells expressing NKp44 in the population of expanded natural killer cells is the same or higher than the percentage of natural killer cells in the seed cells from umbilical cord blood.

60. The method of any one of claims 50-59, wherein the percentage of NK cells expressing NKp46 in the population of expanded natural killer cells is the same or higher than the percentage of natural killer cells in the seed cells from umbilical cord blood.

61. The method of any one of claims 50-60, wherein the percentage of NK cells expressing DNAM-1 in the population of expanded natural killer cells is the same or higher than the percentage of natural killer cells in the seed cells from umbilical cord blood.


SEQUENCE LISTING (22 sequences)

SEQ ID NO: 1 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 223 aa, Homo sapiens
MALPVTALLLPLALLLHAARPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAKPTTTPAPRPPTPAPTIASQPLSLRPEACRPAAGGAVHTRGLDFACDIYIWAPLAGTCGVLLLSLVITLY

SEQ ID NO: 3 - protein, 233 aa, Homo sapiens
MSTESMIRDVELAEEALPKKTGGPQGSRRCLFLSLFSFLIVAGATTLFCLLHFGVIGPQREEFPRDLSLISPLAQPVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 5 - protein, 41 aa, Homo sapiens
RSKRSRLLHSDYMNMTPRRPGPTRKHYQPYAPPRDFAAYRS

SEQ ID NO: 6 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 7 - DNA, 123 bp, Artificial Sequence
  Codon optimized CD28 intracellular signaling domain
cggagcaagaggtcccgcctgctgcacagcgactatatgaacatgaccccacggagacccggccctacacggaaacattaccagccctatgctccaccccgggacttcgcagcttacagaagt

SEQ ID NO: 8 - protein, 22 aa, Homo sapiens
ERVQPLEENVGNAARPRFERNK

SEQ ID NO: 9 - DNA, 66 bp, Homo sapiens
gaaagggtccaacccctggaagagaatgtgggaaatgcagccaggccaagattcgagaggaacaag

SEQ ID NO: 10 - DNA, 66 bp, Artificial Sequence
  Codon optimized OX40L intracellular signaling domain
gaaagagtgcagcccctggaagagaatgtcgggaatgccgctcgcccaagatttgaaaggaacaaa

SEQ ID NO: 11 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 13 - DNA, 336 bp, Artificial Sequence
  Codon optimized CD3zeta signaling domain
cgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 14 - protein, 21 aa, Artificial Sequence
  T2A cleavage site
GSGEGRGSLLTCGDVEENPGP

SEQ ID NO: 15 - DNA, 63 bp, Artificial Sequence
  T2A cleavage site
ggctcaggtgaggggcgcgggagcctgctgacttgtggggatgtagaggaaaatcctggtcct

SEQ ID NO: 16 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 17 - DNA, 489 bp, Homo sapiens
atgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 18 - DNA, 489 bp, Homo sapiens
atgcgcataagtaagcctcatctgcggtccatttctatacaatgttatctgtgcttgcttttgaactcccactttcttacggaagcaggcattcatgtgttcattctgggttgtttttctgccgggctgcccaaaaccgaggccaactgggtcaacgtgatcagcgacctcaagaagatcgaggatttgattcaaagtatgcatatagacgccacactctatactgagtccgacgttcacccgagttgtaaagttacggctatgaagtgctttttgttggaactccaggtgatttcccttgaatccggcgatgcgagcatccacgatacggtagagaatcttattattctggcgaataattctctgtcttcaaatgggaatgtaactgagagcggttgtaaagaatgcgaagaacttgaagaaaagaatatcaaggaatttcttcagagtttcgtgcatattgttcaaatgttcatcaacacatcctga

SEQ ID NO: 19 - protein, 175 aa, Artificial Sequence
  CD28/OX40L/CD3zeta
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 358 aa, Artificial Sequence
  CD28/OX40L/CD3zeta/T2A/IL1-5
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 21 - protein, 609 aa, Homo sapiens [sequence omitted]

SEQ ID NO: 22 - DNA, 603 bp, Artificial Sequence
  Puromycin Resistance Gene
atggccaccgagtacaagcccacggtgcgcctcgccacccgcgacgacgtcccccgggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga